(19) United States
(12) Patent Application Publication
(10) Pub. No.: US 2023/0352114 A1
(43) Pub. Date: Nov. 2, 2023

(54) METHODS OF PROVIDING MODULATORS OF TAU AGGREGATION

(71) Applicant: GTInvent Limited, Aberdeen, Scotland (GB)

(72) Inventors: Michael Philip Mazanetz, Newlands, Glasgow (GB); Claude Michel Wischik, Aberdeen Aberdeenshire (GB); Louise Charlotte Serpell, Falmer Sussex (GB); John Mervyn David Storey, Old Aberdeen Aberdeenshire (GB)

(73) Assignee: GTInvent Limited, Aberdeen, Scotland (GB)

(21) Appl. No.: 18/014,333

(22) PCT Filed: Jul. 6, 2021
(86) PCT No.: PCT/EP2021/068718

§ 371 Date: Jan. 3, 2023

(30) Foreign Application Priority Data
Jul. 10, 2020 (GB) 2010679.5

(51) Int. Cl. G16B 15/30; G16B 15/20; G16B 40/20

(52) U.S. Cl. CPC G16B 15/30; G16B 15/20; G16B 40/20

(57) ABSTRACT

The present invention relates generally to methods for selecting or designing a compound for modulating the aggregation of a Tau protein. The method comprising using computer-implemented molecular modelling means to compare the three-dimensional structure of a candidate compound with a three-dimensional structure of at least a part of the Tau protein comprising amino acids 315-378 and determine whether the candidate compound is able to simultaneously form non-covalent interactions with two or more of Leu315, Ser341, Glu342, Lys343, Phe346, Lys347, Val350, Ser352, Ile354, Lys369, Ile371, Glu372, Phe378 and Thr373. A candidate compound that is able to form said interactions is predicted to modulate the aggregation of the Tau protein or truncated form thereof. Methods using a three-dimensional structural model of at least a part of the Tau protein comprising amino acids 315-378, wherein the model is an intermediate in the aggregation process of the part of the Tau protein with a paired helical filament (PHF) are also described, as are computing systems and products.

### CROSS-REFERENCE TO RELATED APPLICATIONS

This Application is a National Stage filing under 35 U.S.C. § 371 of International PCT Application No. PCT/EP2021/068718, filed Jul. 6, 2021, which claims priority to Great Britain Application No. 2010679.5, filed Jul. 10, 2020. The contents of these applications are incorporated herein by reference in their entirety.

### TECHNICAL FIELD

The present invention relates generally to methods of using the structure coordinates of a Tau binding pocket. In particular, the invention provides a three-dimensional model of a Tau binding pocket and means for identifying, selecting and/or designing modulators of Tau aggregation by rational drug design. The invention also relates generally to using the structure coordinates of a Tau protein that is an intermediate in the process of folding of the Tau protein from a compact conformation comprising the binding pocket to an aggregated confirmation in a paired helical filament.

### BACKGROUND ART

Disorders related to tau, collectively referred to as neurodegenerative tauopathies (Lee et al., 2001) represent a group of diseases of protein aggregation (Buee et al., 2000). Alzheimer's disease (AD) is part of this group of neurodegenerative diseases. Conditions of dementia such as Alzheimer's disease (AD) are frequently characterised by a progressive accumulation of intracellular and/or extracellular deposits of proteinaceous structures such as β-amyloid plaques and neurofibrillary tangles (NFTs) composed of tau, in the brains of affected patients. The appearance of these lesions largely correlates with pathological neuronal degeneration and brain atrophy, as well as with cognitive impairment (see, e.g., Mukaetova-Ladinska et al., 2000). In AD, tau protein self-assembles to form paired helical filaments (PHFs) and straight filaments that constitute the neurofibrillary tangles within neurons and dystrophic neurites in brain. Protein misfolding to form amyloid fibrils is a hallmark of many different diseases collectively known as the amyloidoses, each of which is characterised by a specific precursor protein (Sipe, 1992).

Tau exists in alternatively-spliced isoforms, which contain three or four copies of a repeat sequence corresponding to the microtubule-binding domain (see, e.g., Goedert, M., et al., 1989). The tau species isolated from proteolytically stable PHF-core preparations from AD brain tissue comprise a mixture of fragments derived from both three- and four-repeat isoforms, but restricted to the equivalent of three repeats, with an N-terminus at residues Ile-297 or His-299 and the C-terminus at residue Glu-391, or at homologous positions in other species (Wischik et al., 1988; Jakes et al., 1991). The present inventors have previously shown that the fragment 297-391 (referred to as dGAE) self-assembles to form Paired helical filament-like structures under specific conditions (Al-Hilaly et al., 2017) and that assembly is enhanced under reducing conditions (in the presence of DTT) or at high concentrations. Assembly is accompanied by a conformational change from random coil in solution to beta sheet structure in the insoluble fraction (Al-Hilaly et al., 2017). Residues 306-378 from this same molecule (referred to as “dGAE73” herein) have been visualised in electron density and confirmed as forming part of the core of the PHF using cryo-electron microscopy analysis of PHFs extracted from AD brain tissue (Fitzpatrick et al., 2017).

However, despite the availability of structural data on amyloid fibrils formed by different proteins and from PHFs, the elucidation of a mechanism of amyloid fibril formation from disjoint monomers remains elusive (Sipe 1992; Chiti & Dobson 2006; Roberts 2007; Kelly 2000). To date there has been no agreed description of the primary stages of nucleation from a pool of disjoint soluble oligomeric species (Roberts 2007, Kodali & Wetzel, 2007; Serio et al., 2000; Modler et al., 2004; Glabe 2008; Meisl et al., 2016). A number of amyloidogenic precursors and oligomeric states have been identified (Campioni et al., 2010; Novo et al., 2018; Kayed et al., 2003; Buccianti et al., 2002 and 2004; Gerson et al., 2016), yet isolation and further characterisation of these structurally heterogeneous and transient aggregates remains a challenge. Evidence of soluble protein oligomers as the primary cause of cell impairment and dysfunction in the pathogenesis of tauopathies has been supported through biochemical and biophysical experiments and in vivo assays (Macdonald et al., 2019; Lasagna-Reeves et al., 2012; Gerson et al., 2016).

The present inventors have shown previously that the methylthioninium (MT) species, which can exist in oxidized (MTC, FIG. 12A) and reduced leuco-MT (LMT; FIG. 12C) forms, is able to act as a tau aggregation inhibitor in cell-free, cell based and tau transgenic mouse models of tau aggregation (Harrington et al., 2015; Wischik et al., 1996; Melis et al., 2015; AI-Hilaly et al., 2018). Recent findings indicate that LMT is the active moiety required for inhibition of aggregation of the core tau unit of the PHF (AI-Hilaly et al., 2018). This finding supports the clinical evidence that the stable reduced salt form of the MT moiety (hydromethylthioninium mesylate, LMTM, FIG. 12B) appears to be effective at a dose 20-fold lower than the minimum effective dose previously identified using the oxidized MT+ form (Wischik et al., 2015; Schelter et al., 2019).

As tau aggregation pathology and cognitive decline are closely associated (Okamura et al., 2014; Chien et al., 2013; Mukaetova-Ladinska et al., 2000; Grober et al., 1999; Wilcock and Esiri, 1982; Duyckaerts et al., 1997; Bancher et al., 1993 & 1996; Arriagada et al., 1992), treatment based on the use of inhibitors of tau aggregation offers a potentially promising therapeutic approach for AD (Wischik et al., 2018). A fundamental component for the development of new chemical entities for the treatment of neurodegenerative tauopathies is an understanding of the structure of key intermediates in this aggregation process, and how compounds such as LMT can interfere with this process. This would enable the development of a structure-based hypothesis for ligand design.

### DISCLOSURE OF THE INVENTION

In an attempt to address the above-mentioned needs, the present inventors devised multistep computational protocols to explore the conformational flexibility of a previously reported truncated tau fragment corresponding to one of the species isolated from proteolytically stable PHF preparations (residues ²⁹⁷Ile-Glu³⁹¹) and referred to as “dGAE” (Wischik et al., 1988; Novak et al., 1991; Novak et al., 1993). dGAE assembles spontaneously in physiological conditions in vitro into filaments that closely resemble native PHFs and straight filaments from AD brain morphologically (AI-Hilaly et al., 2017; AI-Hilaly et al., 2020). The analyses were aimed at understanding the flexibility of dGAE, its aggregation into PHFs and the potential of small molecules to inhibit PHF assembly and to cause PHF disaggregation in vivo. In an effort to understand the mechanism of how small molecules could inhibit dGAE assembly into fibrils, the conformation space of a single Tau97 monomer (comprising dGAE and preceding residues ²⁹⁵Asp²⁹⁶Asn) was evaluated with atomistic molecular simulation. The inventors hypothesized that LMT could complex to dGAE monomers and prevent fibril formation. To test this hypothesis, representative protein structures were sampled from the conformational ensemble of apo Tau97. From this, the inventors were able to identify a unique cryptic pocket which would bind to LMT and form a stabilised complex. From the conformation of the complex a pharmacophore model was built and a series of de novo designed compounds were synthesised in order to assess whether stabilising dGAE oligomers would result in inhibition of Tau aggregation. Compounds were assessed in a cell-based tau aggregation assay. More than half of the assayed molecules showed sub micro molar activity. From this, the inventors were able to rationalise the structure-activity relationship from this series of Tau assembly inhibitors.

Thus, the present invention is based, in part, on the discovery of a ligand binding pocket in the Tau protein structure, wherein binding of a ligand in the pocket stabilises the Tau protein in a conformation that is not prone to aggregation into paired helical filaments (PHFs). Interactions between the ligand and any of residues Leu315, Ser341, Glu342, Lys343, Phe346, Lys347, Val350, Ser352, Ile354, Lys369, Ile371, Glu372, Phe378 and Thr373 of Tau were found to be particularly important in stabilising the ligand bound conformation.

The formation of PHF was then analysed through immunostaining dot blots and ELISA assays. These showed that in vitro assembly of PHFs is associated with loss of immunoreactivity with antibodies directed against the PHF core. Furthermore, loss of immunoreactivity could be prevented by including LMT during assembly. Starting from the stabilised conformation of dGAE73 (residues 306-378), a molecular simulation was performed to examine assembly onto a preformed PHF. This simulation supported findings from the dot blots for the assembly of PHFs from stabilized dGAE oligomers. The simulation identified 3 key stages in assembly of PHFs. The first is the electrostatic attraction of oligomers to PHFs and the anchoring of a hairpin turn. This is followed by a proline trans-cis-trans switch which allows for the final step of the formation of stabilising cross-β sheets through hydrophobic zippering of the N- and C-terminal tails of the protein structure. Taken together, the structure-activity results and the dot blots provide strong evidence for the binding of small molecules to the cryptic pocket proposed in our dGAE stabilised conformation, which finds uses in a structure-based approach to design further inhibitors of tau assembly.

Thus the present invention is based, in other aspects, on the discovery of a folding process through which the Tau protein in the compact folded state that includes the binding pocket aggregates with a paired helical filament, where binding of a ligand that inhibits the formation of intermediates in the folding process may stabilise the Tau protein in a conformation that is not prone to aggregation into PHFs. A series of conformational changes from a compact folded state to an aggregated state were found to be particularly important. These conformational changes are such that:
(i) residues Val337-Gln355 form a hairpin loop that moves to align with alternating positively charged and negatively charged sidechain stacks in the hairpin loop of the PHF;
(ii) residue Pro332 switches between a trans and a cis configuration;
(iii) residues 355-378 and 306-318 move to form stabilising cross-β sheets with corresponding residues of the PHF through hydrophobic zippering.

In embodiments, residue Pro332 switching between a trans and a cis configuration causes residues His329, His330 and Lys331 to move close enough to PHF to establish interactions with the partner residues in the next layer of the PHF stack through hydrophobic stacking and a strong hydrogen bond between the side chains of His330 in a preformed layer of the PHF stack and Thr361 of the newly formed dGAE layer on the PHF stack.

In embodiments, step (iii) comprises the following steps: (iii)(1) residues 355-360 move to establish a zipper with the corresponding anti-parallel residues of the PHF; (iii)(2) residues 361-367 move to establish a zipper with the corresponding anti-parallel residues of the PHF; and (iii)(3) residues 306-318 and 368-378 move to form a cross-beta sheet conformation with corresponding residues of the PHF.

According to a first aspect, there is provided a method for selecting or designing a compound for modulating the aggregation of a Tau protein or a truncated form thereof, the method comprising using computer-implemented molecular modelling means to:
compare the three-dimensional structure of a candidate compound with a three-dimensional structure of at least a part of the Tau protein comprising amino acids 315-378 of SEQ ID NO:1 or a variant or derivative thereof that is structurally equivalent to said part; and
determine whether the candidate compound is able to simultaneously form non-covalent interactions with two or more of Leu315, Ser341, Glu342, Lys343, Phe346, Lys347, Val350, Ser352, Ile354, Lys369, Ile371, Glu372, Phe378 and Thr373, of SEQ ID NO:1, or equivalent amino acids in a variant or derivative, wherein a candidate compound that is able to form said interactions is predicted to modulate the aggregation of the Tau protein or truncated form thereof.

According to a related aspect, there is provided a method for selecting or designing a compound for modulating the aggregation of a Tau protein or a truncated form thereof, the method comprising using computer-implemented molecular modelling means to:
compare the three-dimensional structure of a candidate compound with a three-dimensional structure of at least a part of the Tau protein comprising amino acids 315-378 of SEQ ID NO:1 as defined in Table 1, or a variant or derivative thereof that is structurally equivalent to said part; and
determine whether the candidate compound is able to form non-covalent interactions with the part of the Tau protein having the structure defined in Table 1 which stabilise said structure, wherein a candidate compound that is able to form said interactions is predicted to modulate the aggregation of the Tau protein or truncated form thereof.

As used herein, the step of comparing the three-dimensional structure of a candidate compound with a three-dimensional structure of at least a part of the Tau protein comprises providing the three-dimensional structure of the candidate compound, providing the three-dimensional structure of the part of the Tau protein, and obtaining a molecular model that includes both three-dimensional structures.

In embodiments, the non-covalent interactions comprise interactions with one or more, optionally with two or more of: Leu315, Ser341, Glu342, Lys343, Phe346, Lys347, Val350, Ser352, Lys369, Ile371, Glu372, and Thr373, of SEQ ID NO:1, or equivalent amino acids in a variant or derivative. Non-covalent interactions with the above-mentioned residues of the Tau protein were found to stabilise a compact conformation of the Tau protein (an example of which is provided by the coordinates defined in Table 1), reducing the propensity of the Tau protein to aggregate.

In embodiments, the non-covalent interactions comprise at least a non-covalent interaction with Lys347. Indeed, a potent inhibitor of Tau aggregation, compound 16, was identified by the inventors as binding the Tau protein by establishing an interaction with Lys347, thereby stabilising a conformation of the protein that has reduced propensity to aggregate. Further potent inhibitors of Tau aggregation were also found to interact with Lys347, including LMT, compound 17, compound 12, compound 1, compound 7, compound 5, compound 14, compound 2, compound 8, compound 13 and compound 18.

In embodiments, the non-covalent interactions comprise at least a non-covalent interaction with Lys343. Indeed, a potent inhibitor of Tau aggregation, compound 11, was identified by the inventors as binding the Tau protein by establishing an interaction with Lys343, thereby stabilising a conformation of the protein that has reduced propensity to aggregate. Further potent inhibitors of Tau aggregation were also found to interact with Lys343, including LMT, compound 17, compound 12, compound 3, compound 14, compound 10, compound 13 and compound 18.

In embodiments, the non-covalent interactions comprise at least a non-covalent interaction with Thr373. Indeed, a potent inhibitor of Tau aggregation, compound 9, was identified by the inventors as binding the Tau protein by establishing an interaction with Thr373, thereby stabilising a conformation of the protein that has reduced propensity to aggregate. Further potent inhibitors of Tau aggregation were also found to interact with Thr373, including LMT, compound 3, compound 4, and compound 15.

In embodiments of any aspect, the non-covalent interactions comprise an interaction with two or more of Lys343, Leu315, Lys347, Glu372, and Thr373, of SEQ ID NO:1.

In embodiments of any aspect, the non-covalent interactions comprise an interaction with one or more of Leu315, Val350, and Ile371 of SEQ ID NO:1. Interactions with these residues are preferably hydrophobic interactions, involving the side chain of the residue. Indeed, multiple potent inhibitors of Tau aggregations, such as compounds 16, 9 and 11, were identified by the inventors as binding the Tau protein by establishing an interaction with these residues.

In embodiments of any aspect, the non-covalent interactions comprise an interaction with two or more (such as all of) Lys347, Lys343, Ser352 and Thr373 of SEQ ID NO: 1. A known inhibitor of Tau aggregation, LMT was identified by the inventors as binding the Tau protein in a cryptic binding pocket, where it establishes interactions with the above-mentioned residues, thereby stabilising a conformation of the protein that has reduced propensity to aggregate.

In embodiments of any aspect, the non-covalent interactions comprise an interaction with two or more (such as all of) Lys347, Lys343, Ser341, Leu315, Ile371, Glu372 and Phe346 of SEQ ID NO: 1. A potent inhibitor of Tau aggregation, compound 17, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Lys347, Lys343, Ile371, and Glu372 of SEQ ID NO: 1. A potent inhibitor of Tau aggregation, compound 12, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Lys347, Ile371, Glu342, Phe346 and Glu372 of SEQ ID NO: 1. A potent inhibitor of Tau aggregation, compound 1, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Leu315, Lys347, Ile371, Glu342, Phe346 and Glu372 of SEQ ID NO: 1. A potent inhibitor of Tau aggregation, compound 7, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with at least Lys343 and Thr373 of SEQ ID NO: 1. A potent inhibitor of Tau aggregation, compound 3, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Glu372, Lys369, and Thr373 of SEQ ID NO: 1. A potent inhibitor of Tau aggregation, compound 7, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Lys343, Lys347, Ile371, Glu342, Phe346 and Glu372 of SEQ ID NO: 1. A potent inhibitor of Tau aggregation, compound 5, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Leu315, Lys347, and Lys343 of SEQ ID NO: 1. A potent inhibitor of Tau aggregation, compound 14, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Lys343, Lys347, and Glu372 of SEQ ID NO: 1. A potent inhibitor of Tau aggregation, compound 2, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Lys347, Glu342, Phe346 and Glu372 of SEQ ID NO: 1. A potent inhibitor of Tau aggregation, compound 8, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Thr373, Lys369, and Lys343 of SEQ ID NO: 1. A potent inhibitor of Tau aggregation, compound 10, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Leu315, Ile371, and Lys343 of SEQ ID NO: 1. A potent inhibitor of Tau aggregation, compound 6, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Ile371, Glu372, Lys343, Phe346, and Lys347 of SEQ ID NO: 1. A potent inhibitor of Tau aggregation, compound 13, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Glu372 and Thr373 of SEQ ID NO: 1. A potent inhibitor of Tau aggregation, compound 15, was identified by the inventors as binding the Tau protein by establishing interactions with the above-mentioned residues, thereby stabilising a conformation of the protein that has reduced propensity to aggregate.

In embodiments of any aspect, the non-covalent interactions comprise an interaction with two or more (such as all of) Lys343, Phe346, and Lys347 of SEQ ID NO: 1. A potent inhibitor of Tau aggregation, compound 18, was identified by the inventors as binding the Tau protein by establishing interactions with the above-mentioned residues, thereby stabilising a conformation of the protein that has reduced propensity to aggregate.

The methods of the first and second aspect may comprise determining whether the candidate compound is able to simultaneously form non-covalent molecular interactions with Lys343 and Glu372 of SEQ ID NO:1.

Multiple compounds found to have a strong effect in modulating Tau aggregation were predicted to form stabilising interactions with Lys343 and Glu372, stabilising the Tau protein in a conformation that include the binding pocket exposing these residues.

In embodiments, a non-covalent molecular interaction with Lys343 comprises a cation-pi interaction and/or a hydrogen bond and/or a pi-H interaction. In some embodiments, a cation-pi interaction with Lys343 is between an aromatic ring of the candidate compound and the sidechain amino group of Lys343. In some embodiments, a hydrogen bond with Lys343 is between an acceptor group of the candidate compound and the backbone amino group of Lys343. In some embodiments, a hydrogen bond with Lys343 is between a donor group of the candidate compound and the backbone carbonyl of Lys343. In some embodiments, a pi-H interaction with Lys343 is between an aromatic ring of the candidate compound and the sidechain Cp of Lys343. In some embodiments, a pi-H interaction with Lys343 is between an aromatic ring of the candidate compound and the backbone amino group of Lys343. In some embodiments, a pi-H interaction with Lys343 is between an aromatic ring of the candidate compound and the sidechain CE of Lys343.

In embodiments, a non-covalent interaction with Ser352 comprises a pi-H interaction. In some such embodiments, a pi-H interaction with Ser352 is between an aromatic ring of the candidate compound and the backbone carbonyl of Ser352.

In embodiments, the non-covalent interaction with any of Leu315, Ser341, Phe346, Lys347, Ile371, Glu372, and Thr373, of SEQ ID NO:1 is a hydrogen bond.

In embodiments, the non-covalent interaction with Glu372 is a hydrogen bond. In some such embodiments, a hydrogen bond with Glu372 is between a H donor group of the candidate compound and the sidechain carboxyl (OE1) of Glu372. In some embodiments, a hydrogen bond with Glu372 is between an acceptor group of the candidate compound and the backbone amino group of Glu372. In some embodiments, a hydrogen bond with Glu372 is between an acceptor group of the candidate compound and the sidechain amino group of Glu372.

In embodiments, the non-covalent interaction with Lys347 is a hydrogen bond. In embodiments, the hydrogen bond with Lys347 is between an acceptor group of the candidate compound and the backbone amino group of Lys347.

In embodiments, the non-covalent interaction with Thr373 is a hydrogen bond. In embodiments, a hydrogen bond with Thr373 is between a donor group of the candidate compound and the hydroxyl group of the side-chain of Thr373.

In embodiments, the non-covalent interaction with Leu315 is a hydrogen bond. In embodiments, a hydrogen bond with Leu315 is between a donor group of the candidate compound and the backbone carbonyl of Leu315.

In embodiments, the candidate compound is able to form a non-covalent interaction with Phe378. In embodiments, the non-covalent interaction is a pi-interaction.

In embodiments, the non-covalent interaction with Ile371 is a hydrogen bond. In embodiments, a hydrogen bond with Ile371 is between an acceptor group of the candidate compound and the backbone Ca of Ile371.

In embodiments, the non-covalent interaction with Ser341 is a hydrogen bond. In embodiments, a hydrogen bond with Ser341 is between an acceptor group of the candidate compound and the sidechain Cp of Ser341.

In embodiments, the non-covalent interaction with Phe346 is a hydrogen bond. In embodiments, a hydrogen bond with Phe346 is between an acceptor group of the candidate compound and the backbone Ca of Phe346.

In embodiments, the non-covalent interaction with Glu342 is a hydrogen bond. In some embodiments, a hydrogen bond with Glu342 is between an acceptor group of the candidate compound and the backbone amino group of Glu342.

In embodiments, the non-covalent interaction with Lys369 is a hydrogen bond. In embodiments, a hydrogen bond with Lys369 is between an acceptor group of the candidate compound and the backbone CE of Lys369. In embodiments, a hydrogen bond with Lys369 is between an acceptor group of the candidate compound and the backbone amino group of Lys369.

In embodiments, the interactions with any of Val350, Leu315, Ile354, Ile371, Phe378 and Phe346 are hydrophobic interactions.

In embodiments, the compound is for inhibiting the aggregation of a Tau protein or a truncated form thereof into paired helical filaments.

The compound may be a small molecule, a peptide, a polypeptide or a combination thereof. Preferably, the compound is a small molecule.

In embodiments, the three-dimensional structure of the part of the Tau protein comprising amino acids 315-378 of SEQ ID NO:1 comprises a binding pocket that is capped by Phe378 and Phe346, exposes the hydrophobic side chains of residues Val350, Leu315, Ile354 and/or Ile371, and contains residues Leu315, Lys343, Lys347, Glu372, and/or Thr373 capable of forming hydrogen bonds to a molecule within the binding pocket.

In embodiments, the candidate compound is able to simultaneously form non-covalent molecular interactions with one or more of residues Leu315, Ser341, Glu342, Lys343, Phe346, Lys347, Ser352, Lys369, Ile371, Glu372, and Thr373, of SEQ ID NO:1, or equivalent amino acids in a variant or derivative, without exposing hydrophilic groups at a distance below 4 Å from the hydrophobic side chains of residues Val350, Leu315, Ile354 and Ile371.

In embodiments, the three-dimensional structure of the part of the Tau protein comprising amino acids 315-378 of SEQ ID NO:1 comprises a hairpin loop between residues Val337 and Gly355 of SEQ ID NO:1.

The present inventors have identified that a compact folded state that is able to interact with inhibitors of Tau aggregation such as LMT, and is stabilised by this interaction, comprises a hair pin loop between residues Val337 and Gly355 of SEQ ID NO:1.

In embodiments, the three-dimensional structure of the part of the Tau protein comprising amino acids 315-378 of SEQ ID NO:1 comprises the sequence Pro364-Gly367 located between a loop formed by the sequence Tyr219-Lys331 and the sequence Pro332-Gly335.

In embodiments, the three-dimensional structure of the part of the Tau protein comprising amino acids 315-378 of SEQ ID NO:1 is stabilised at least in part by hydrogen bonds between Glu342 and Val318 and/or Thr319, between Gly367 and Lys340, between Asn368 and Lys340, between Lys369 and Glu372, between Lys370 and Asp358, between Ile371 and Ser316, between Glu372 and Ser356, between Thr373 and Gln351, Ser316, between His374 and Gln351, and between Lys375 and Gln351.

The three-dimensional structure of the part of the Tau protein comprising amino acids 315-378 of SEQ ID NO:1 may be that represented by the structure co-ordinates in Table 1, or a structure modelled on these coordinates.

The three-dimensional structure of the part of the Tau protein may be obtainable by performing molecular dynamics simulations of a part of the Tau protein comprising amino acids 315-378 of SEQ ID NO:1 in the presence of LMT to obtain one or more complex conformations and selecting a three-dimensional structure of the part of the Tau protein by applying a stability criterion and a binding affinity criterion to the one or more complex conformations.

Advantageously, the stability criterion may apply to the distance between complex conformations in consecutive frames of the molecular dynamics simulation after a predetermined amount of time, and/or the binding affinity criterion may apply to the value of a placement scoring function <−80 kcal/mol or a scoring function <−8.5 as determined by the GB IV scoring function within the CCG MOE docking software.

In embodiments, the stability criterion applies to the root-mean-square deviation (RMSD) of atomic positions, for atoms in the backbone of the part of protein in the complexes, between consecutive frames of the molecular dynamics simulation.

In embodiments, the stability criterion applies to the root-mean-square deviation (RMSD) of atomic positions, for atoms in LMT. In embodiments, a stability criterion may be defined as a threshold on the average RMSD of atomic positions, for atoms in the backbone of the part of protein in the complexes, between consecutive frames of the molecular dynamics simulation, where the average is calculated over a predetermined set of frames of a MD simulation. For example, the predetermined set of frames may be defined as the last 10% of frames, the last 5% of frames, or the last 1% of frames in a MD simulation. The predetermined set of frames may be defined as the last 30 k frames, the least 25 k frames, the last 20 k frames, the last 15 k frames, or the last 10 k frames. The threshold on the average RMSD may be chosen as about 0.5 Å, about 0.6 Å, about 0.7 Å, about 0.8 Å or about about 0.9 Å.

In particular, the present inventors have found that a stable LMT-bound conformation could be obtained by running molecular dynamics simulation of a part of the Tau protein in the presence of LMT, and excluding those complexes where after a predetermined amount of simulation time, the LMT was no longer tightly bound (as indicated by the docking score or ligand RMSD fluctuations) and/or the RMSD fluctuations between consecutive frames of the simulation indicated that the conformation was not stable.

In embodiments, the predetermined amount of time is at least 50 ns, at least 100 ns, or about 100 ns.

In embodiments, the methods described herein further comprises designing a pharmacophore model, wherein the pharmacophore includes features representative of non-covalent molecular interactions with two or more of: Leu315, Ser341, Glu342, Lys343, Phe346, Lys347, Val350, Ser352, Ile354, Lys369, Ile371, Glu372, Phe378 and Thr373, of SEQ ID NO:1.

In embodiments, designing a pharmacophore model comprises computing the interaction energy between the three-dimensional structure of each candidate compound in a library of candidate compounds and the three-dimensional structure of at least a part of the Tau protein comprising amino acids 315-378 of SEQ ID NO:1 or a variant or derivative thereof that is structurally equivalent to said region, selecting a subset of the library that has an interaction energy below a threshold, and deriving a pharmacophore model based on the selected subset.

The part of the Tau protein may comprise amino acids 306-378 of SEQ ID NO:1. The part of the Tau protein may comprise amino acids 297-391 of SEQ ID NO:1. The part of the Tau protein may comprise amino acids 295-391 of SEQ ID NO:1. The part of the Tau protein may consist of amino acids 297-391 of SEQ ID NO:1. The part of the Tau protein may consist of amino acids 295-391 of SEQ ID NO:1. The part of the Tau protein may consist of amino acids 306-378 of SEQ ID NO:1. In embodiments, the part of the Tau protein comprises amino acids 306-378 of SEQ ID NO:1 (also referred to herein as dGAE73, SEQ ID NO: 4) or a variant or derivative thereof that is structurally equivalent to said part.

In embodiments, the part of the Tau protein comprises amino acids 297-391 of SEQ ID NO:1 (also referred to herein as dGAE, SEQ ID NO: 3) or a variant or derivative thereof that is structurally equivalent to said part. In embodiments, the part of the Tau protein comprises amino acids 295-391 of SEQ ID NO:1 (also referred to herein as Tau97, SEQ ID NO: 5) or a variant or derivative thereof that is structurally equivalent to said part.

In embodiments, the part of the Tau protein comprises the full sequence of the human Tau isoform 2N4R (SEQ ID NO: 1) or a homolog or variant thereof. In embodiment, a variant has at least 90% amino acid identity with the sequence of SEQ ID NO: 1, or the portion thereof that is represented in the three-dimensional structure.

The methods may further comprise repeating the steps of comparing and determining with a further candidate compound that differs from the previous candidate compound in at least one substituent.

In embodiments, one or more candidate compounds may be modelled such that their properties can be compared to thereby define the features that advantageously stabilise the ligand-bound conformation of the part of the Tau protein. Comparing the properties of multiple candidate compounds may comprise comparing, using computational molecular modelling means, the characteristics of the complexes comprising the part of the Tau protein and each of the candidate compounds in terms of e.g. stability, binding affinity, etc. Comparing the properties of multiple candidate compounds may comprise performing one or more experiments to quantify the Tau aggregation modulation associated with each candidate compound.

In embodiments, such comparisons may be performed sequentially to progressively optimise a candidate compound, where the information obtained by comparing a candidate compound to a previously obtained candidate compound is used to design a new candidate compound in a subsequent iteration. Instead or in addition to this, such comparisons may be performed in parallel where multiple candidate compounds are simultaneously compared to derive insights from common properties of subsets of candidate compounds tested. Such insights can be used to design one or more further candidate compounds.

Without wishing to be bound by theory, it is believed that such a process may help to define the features of a candidate compound that optimally modulates Tau aggregation by stabilising a compact folded conformation of the Tau protein.

Comparing the three-dimensional structure of a candidate compound with a three-dimensional structure of at least a part of the Tau protein comprising amino acids 315-378 of SEQ ID NO:1 or a variant or derivative thereof that is structurally equivalent to said region may comprise computing the interaction energy between the candidate compound and the part of the Tau protein represented in the three-dimensional structure.

According to a further aspect, there is provided a computer-implemented method for evaluating the ability of a candidate compound to bind to a binding pocket of a Tau protein, the method comprising the steps of:
(a) receiving the three-dimensional structure coordinates of a part of the Tau protein comprising amino acids 315-378 of SEQ ID NO:1 or equivalent amino acids in a variant or derivative thereof that is structurally equivalent to said part, wherein the three-dimensional structure of amino acids 315-378 of SEQ ID NO: 1 comprises the binding pocket of the Tau protein;
(b) performing a fitting operation between a candidate compound and the binding pocket; and
(c) analysing the results of the fitting operation to determine whether the candidate compound is able to bind to the binding pocket.

In embodiments, step (c) comprises determine whether the candidate compound is able fit at least in part within the binding pocket and form non-covalent molecular interactions with one or more of Lys343, and at least one of Leu315, Ser341, Glu342, Lys343, Phe346, Lys347, Val350, Ser352, Ile354, Lys369, Ile371, Glu372, Phe378 and Thr373, of SEQ ID NO:1, or equivalent amino acids in a variant or derivative.

In embodiments, the three-dimensional structure of the part of the Tau protein comprising amino acids 315-378 of SEQ ID NO:1 is that represented by the structure co-ordinates shown in Table 1, or a structure modelled on these coordinates.

In embodiments, the compound is a small molecule, a peptide, a polypeptide or a combination thereof.

In embodiments, a candidate compound is considered able to bind to the binding pocket if it is able to establish non-covalent interactions with amino acids in the binding pocket. In embodiments, the non-covalent interactions are selected from: hydrophobic interactions, hydrogen bonds, pi-cation interactions, and pi-stack interactions.

In embodiments, a candidate compound that is considered able to bind to the binding pocket is predicted to inhibit the aggregation of the Tau protein or a truncated form thereof. In embodiments, a candidate compound that is considered able to bind to the binding pocket is predicted to inhibit the aggregation of the Tau protein or a truncated form thereof into paired helical filaments.

In embodiments, the method further comprises evaluating the ability of a further candidate compound to bind the Tau protein at a different site from the binding site of the first candidate compound.

In embodiments, evaluating the ability of a further candidate compound to bind the Tau protein at a different site from the binding site of the first candidate compound comprises computing the interaction energy between the further candidate compound and the three-dimensional structure of the part of the Tau protein and determining whether the interaction between the further candidate compound and the Tau protein further stabilises the conformation of the complex between the first candidate compound and the Tau protein.

In embodiments, the first candidate compound is a small molecule or a peptide, and the further candidate compound is a peptide or a polypeptide, such as an antibody or fragment thereof.

In embodiments, the candidate compound is able to bind to the pocket if it is able to simultaneously form non-covalent molecular interactions with two or more of Leu315, Ser341, Glu342, Lys343, Phe346, Lys347, Val350, Ser352, Ile354, Lys369, Ile371, Glu372, Phe378 and Thr373, of SEQ ID NO:1.

In embodiments, the candidate compound is able to bind to the pocket if it is able to simultaneously form non-covalent molecular interactions with Lys343 and at least one of Leu315, Lys347, Glu372 and Thr373.

In some cases, the candidate compound is able to bind to the pocket if it is able to simultaneously form non-covalent molecular interactions with Lys343 and Glu372 of SEQ ID NO:1.

In embodiments, the binding pocket is capped by Phe378 and Phe346, exposes the hydrophobic side chains of residues Val350, Leu315, Ile354 and/or Ile371, and contains residues Leu315, Lys343, Lys347, Glu372, and/or Thr373 capable of forming hydrogen bonds to a molecule within the binding pocket.

In embodiments, the three-dimensional structure of the part of the Tau protein comprising amino acids 315-378 of SEQ ID NO:1 comprises a hairpin loop between residues Val337 and Gly355 of SEQ ID NO:1.

In embodiments, the three-dimensional structure of the part of the Tau protein comprising amino acids 315-378 of SEQ ID NO:1 comprises the sequence Pro364-Gly367 located between a loop formed by the sequence Tyr219-Lys331 and the sequence Pro332-Gly335.

In embodiments, the three-dimensional structure of the part of the Tau protein comprising amino acids 315-378 of SEQ ID NO:1 is stabilised at least in part by hydrogen bonds between Glu342 and Val318, Thr319, between Gly367 and Lys340, between Asn368 and Lys340, between Lys369 and Glu372, between Lys370 and Asp358, between Ile371 and Ser316, between Glu372 and Ser356, between Thr373 and Gln351, Ser316, between His374 and Gln351, between Lys375 and Gln351.

In embodiments, the three-dimensional structure coordinates of a part of the Tau protein comprising amino acids 315-378 of SEQ ID NO:1 or equivalent amino acids in a variant or derivative thereof that is structurally equivalent to said part have been obtained by performing molecular dynamics simulations of a part of the Tau protein comprising amino acids 315-378 of SEQ ID NO:1 in the presence of LMT to obtain one or more complex conformations and selecting a complex conformation using a stability criterion and a binding affinity criterion.

In embodiments, step (a) receiving the three-dimensional structure coordinates of a part of the Tau protein comprises obtaining the three-dimensional structure coordinates of a part of the Tau protein comprising amino acids 315-378 of SEQ ID NO:1 or equivalent amino acids in a variant or derivative thereof that is structurally equivalent to said part by:
(a) performing molecular dynamics simulations of a part of the Tau protein comprising amino acids 315-378 of SEQ ID NO:1 in the presence of LMT to obtain one or more complex conformations that differ in their three-dimensional conformations; and
(b) selecting a complex conformation using a stability criterion and a binding affinity criterion, wherein the three-dimensional structure coordinates of a part of the Tau protein comprising amino acids 315-378 of SEQ ID NO:1 are defined as the three-dimensional structure coordinates of the part of the Tau protein in the selected complex conformation;
optionally wherein the stability criterion applies to the distance between conformations in consecutive frames of the molecular dynamics simulation after a predetermined amount of time, and/or wherein the binding affinity criterion applies to the value of a docking score.

In embodiments, selecting a complex conformation using a stability criterion comprises:
computing the root-mean-square deviation (RMSD) of atomic positions, for atoms in the backbone of the part of protein in the complex conformations, between consecutive frames of the molecular dynamics simulation over a predetermined amount of time; and
selecting the complex conformation(s) that has/have a RMSD below a predetermined threshold and/or that have the lowest RMSD amongst the one or more complex conformations.

In embodiments, the predetermined amount of time is at least 50 ns, at least 100 ns, or about 100 ns.

In embodiments, selecting a complex conformation using a binding affinity criterion comprises computing the interaction energy between the candidate compound and the part of the Tau protein represented in the three-dimensional structure.

In embodiments, performing molecular dynamics simulations of a part of the Tau protein comprising amino acids 315-378 of SEQ ID NO:1 in the presence of LMT comprises obtaining the three-dimensional structure coordinates of a part of the Tau protein comprising amino acids 315-378 of SEQ ID NO:1 or equivalent amino acids in a variant or derivative thereof that is structurally equivalent to said part, wherein the three-dimensional structure coordinates correspond to a conformation of the Tau protein as part of a PHF stack. In embodiments, the three-dimensional structure coordinates correspond to those of PDB ID: 5O3L or a structure modelled on this structure.

The part of the Tau protein may comprise amino acids 306-378 of SEQ ID NO:1. The part of the Tau protein may comprise amino acids 297-391 of SEQ ID NO:1. The part of the Tau protein may comprise amino acids 295-391 of SEQ ID NO:1. The part of the Tau protein may consist of amino acids 297-391 of SEQ ID NO:1. The part of the Tau protein may consist of amino acids 295-391 of SEQ ID NO:1. The part of the Tau protein may consist of amino acids 306-378 of SEQ ID NO:1.

In embodiments, the part of the Tau protein comprises amino acids 306-378 of SEQ ID NO:1 (also referred to herein as dGAE73, SEQ ID NO: 4) or a variant or derivative thereof that is structurally equivalent to said part.

In embodiments, the part of the Tau protein comprises amino acids 297-391 of SEQ ID NO:1 (also referred to herein as dGAE, SEQ ID NO: 3) or a variant or derivative thereof that is structurally equivalent to said part.

In embodiments, the part of the Tau protein comprises amino acids 295-391 of SEQ ID NO:1 (also referred to herein as Tau97, SEQ ID NO: 5) or a variant or derivative thereof that is structurally equivalent to said part.

In embodiments, the part of the Tau protein comprises the full sequence of the human Tau isoform 2N4R (SEQ ID NO: 1) or a homolog or variant thereof. In embodiment, a variant has at least 90% amino acid identity with the sequence of SEQ ID NO: 1, or the portion thereof that is represented in the three-dimensional structure.

In embodiments, the three-dimensional structure coordinates of the part of the Tau protein comprising amino acids 315-378 of SEQ ID NO:1 or equivalent amino acids in a variant or derivative thereof that is structurally equivalent to said part correspond to a conformation that has the following structural characteristics:
hydrogen bonds between Glu342 and Val318 and/or Thr319; optionally wherein the hydrogen bonds are between the Glu342 carboxylic acid and the backbone NH of Val318 and the sidechain OH of Thr319;
one or more hydrogen bonds between one or more of residues Lys369-Thr377 and one or more of residue Ser341-Gln351; optionally wherein the one or more bonds comprise:
   (i) a bond between Gln351 and Thr373, preferably wherein the bond is between the backbone carbonyl of Gln351 and the hydroxyl sidechain of Thr373;
   (ii) a bond between Gln351 and His374, preferably wherein the bond is between the sidechain carbonyl of Gln351 and the sidechain amine of His374;
   (iii) a bond between Gln351 and Lys375, preferably wherein the bond is between the sidechain carbonyl of Gln351 and the backbone amine of Lys375;
   (iv) a bond between Arg349 and Thr377, preferably wherein the bond is between a sidechain amine of Arg349 and the hydroxyl sidechain of Thr377, between a sidechain amine of Arg349 and the hydroxyl backbone of Thr377, and/or between the carbonyl backbone of Arg349 and the hydroxyl sidechain of Thr377;
   (v) a bond between Glu372 and Ser356, preferably wherein the bond is between the carboxylic acid side chain of Glu372 and the backbone NH of Ser356, or between the carboxylic acid side chain of Glu372 and the OH-sidechain of Ser356; and/or
   (vi) a bond between Glu372 and Lys369, preferably wherein the bond is between the carboxylic acid side chain of Glu372 and the NH sidechain of Lys369;
no beta sheets;
a hairpin loop comprising residues Val337-Gly355;
the PGGG sequence formed by residues Pro364-Gly367 is within a distance of 13 A of the PGGG sequence Pro332-Gly335 and/or within a distance of 2 A of a loop formed by the sequence Thr319-Lys331;
the PGGG sequence formed by residues Pro364-Gly367 is located between the PGGG sequence Pro332-Gly335 and a loop formed by the sequence Thr319-Lys331;
residues Lys369-Thr377 are within a distance of 6 A of residues Asp314-Ser316, optionally wherein the distance between the Ser316 beta-carbon and Thr373 backbone carbonyl is between 2.5 Å and 5.0 Å;
residues Gly355-Gly367 and Asn368-Arg379 are within 2 A hydrogen bonding distance;
Glu338 is folded towards Val363, optionally wherein the distance (RMSD) between the carbonyl oxygen side chain of Glu338 and the backbone amine nitrogen of Val363 NH during the final 10 ns of a 50 ns simulation is between 2 Å and 4 Å, or below 5 Å;
the total water accessible surface area calculated for the part of the protein is at least 20% lower than the corresponding values calculated for a conformation as provided by the three-dimensional coordinates with PDB identifier 5O3L; and/or
the polar and/or hydrophobic accessible surface area(s) calculated for the part of the protein is/are at least 20% lower than the corresponding values calculated for a conformation as provided by the three-dimensional coordinates with PDB identifier 5O3L.

In embodiments, the three-dimensional structure coordinates of the part of the Tau protein is such that the distance (RMSD) between the backbone carbonyl oxygen of Gln351 and the sidechain hydroxyl oxygen of Thr373 during the final 10 ns of a 50 ns simulation is between 2 Å and 4 Å, or below 5 Å.

In embodiments, the three-dimensional structure coordinates of the part of the Tau protein is such that the distance (RMSD) between the sidechain carbonyl oxygen of Gln351 and the sidechain amine nitrogen of His374 during the final 10 ns of a 50 ns simulation is between 2 Å and 4 Å, or below 5 Å.

In embodiments, the three-dimensional structure coordinates of the part of the Tau protein is such that the distance (RMSD) between the sidechain carbonyl oxygen of Gln351 and the backbone amine nitrogen of Lys375 during the final 10 ns of a 50 ns simulation is between 2.5 Å and 5 Å, or below 5 Å.

In embodiments, the three-dimensional structure coordinates of the part of the Tau protein is such that the distance (RMSD) between the backbone carbonyl oxygen of Arg349 and the hydroxyl sidechain oxygen of Thr377 during the final 10 ns of a 50 ns simulation is between 2.5 Å and 4 Å, or below 5 Å.

In embodiments, the three-dimensional structure coordinates of the part of the Tau protein is such that the distance (RMSD) between the carboxylic acid sidechain oxygen of Glu372 and the backbone NH of Ser356 during the final 10 ns of a 50 ns simulation is between 2 Å and 4 Å, or below 5 Å.

According to a further aspect, there is provided a method for selecting or designing a compound for modulating the aggregation of a Tau protein or a truncated form thereof, the method comprising:
using a three-dimensional structural model of at least a part of the Tau protein comprising amino acids 315-378 of SEQ ID NO:1 or a variant or derivative thereof that is structurally equivalent to said part, wherein the model is an intermediate in the aggregation process of the part of the Tau protein with a paired helical filament (PHF), wherein the model is generated by simulating the conformational changes of the part of the Tau protein from a compact folded state to a an aggregated state such that:
   (i) residues Val337-Gln355 form a hairpin loop that moves to align with alternating positively charged and negatively charged sidechain stacks in the hairpin loop of the PHF;
   (ii) residue Pro332 switches between a trans and a cis configuration;
   (iii) residues 355-378 and 306-318 move to form stabilising cross-β sheets with corresponding residues of the PHF through hydrophobic zippering; and generating a model of a complex of the compound and the intermediate.

In embodiments, residue Pro332 switching between a trans and a cis configuration causes residues His329, His330 and Lys331 to move close enough to PHF to establish interactions with the partner residues in the next layer of the PHF stack through hydrophobic stacking and a strong hydrogen bond between the side chains of His330 in a preformed layer of the PHF stack and Thr361 of the newly formed dGAE layer on the PHF stack.

In embodiments, step (iii) comprises the following steps: (iii)(1) residues 355-360 move to establish a zipper with the corresponding anti-parallel residues of the PHF; (iii)(2) residues 361-367 move to establish a zipper with the corresponding anti-parallel residues of the PHF; and (iii)(3) residues 306-318 and 368-378 move to form a cross-beta sheet conformation with corresponding residues of the PHF.

In embodiments, residues 355-360 move to establish a zipper with the corresponding anti-parallel residues of the PHF from the C- to N-direction.

In embodiments, residues 361-367 move to establish a zipper with the corresponding anti-parallel residues of the PHF in the N- to C-direction.

In embodiments, the cross-beta sheet conformation is formed simultaneously starting from Phe378 and closing the residues from C- to N-terminal, and joining residues 318-306 of the N-terminal in the C- to N-direction.

In embodiments, the compound is a small molecule, a peptide, a polypeptide or a combination thereof. In embodiments, the polypeptide is an antibody or a fragment thereof.

In embodiments, the method further comprises selecting or designing a further compound for modulating the aggregation of a Tau protein or a truncated form thereof by generating a model of a complex of the first compound, the further compound and the intermediate.

In embodiments, the further compound is a small molecule, a peptide, a polypeptide or a combination thereof. In embodiments, the polypeptide is an antibody or a fragment thereof.

The compact folded state may have any of the structural characteristics defined in relation to the preceding aspect.

The compact folded state may have the structure co-ordinates shown in Table 1, or a structure modelled on these coordinates.

Generating a model of a complex of the compound and the intermediate may comprise identifying the compound as binding to the intermediate and preventing the occurrence of any of steps (i) to (v).

Generating a model of a complex of the compound and the intermediate may comprise identifying the compound as binding to the intermediate and preventing the occurrence of any of steps (i) to (iii).

In embodiments, simulating the conformational changes of the part of the Tau protein from a compact folded state to an aggregated state comprises performing a molecular dynamics simulation of the assembly of a Tau protein or a part thereof with a PHF stack. In embodiments, the PHF stack comprises at least 2, at least 4, at least 6, at least 8 or at least 10 monomers. In embodiments, the PHF stack comprises the structure defined in PDB ID: 5O3L, or a structure modelled from the structure in PDB ID: 5O3L.

In embodiments, the part of the Tau protein comprises amino acids 306-378 of SEQ ID NO:1 (also referred to herein as dGAE73, SEQ ID NO: 4) or a variant or derivative thereof that is structurally equivalent to said part.

In embodiments, the part of the Tau protein comprises amino acids 297-391 of SEQ ID NO:1 (also referred to herein as dGAE, SEQ ID NO: 3) or a variant or derivative thereof that is structurally equivalent to said part.

In embodiments, the part of the Tau protein comprises amino acids 295-391 of SEQ ID NO:1 (also referred to herein as Tau97, SEQ ID NO: 5) or a variant or derivative thereof that is structurally equivalent to said part.

In embodiments, simulating the conformational changes of the part of the Tau protein from a compact folded state to an aggregated state comprises performing a first set of molecular dynamics simulation of the assembly of a Tau protein or a part thereof with a PHF stack, in which the anchoring of the part of the Tau protein to the PHF stack is determined by starting the molecular dynamics simulations in the set with different relative orientations of the part of the Tau protein and the PHF stack.

In embodiments, the molecular dynamics simulation in the set are each started with a random orientation of the part of the Tau protein. Preferably, the molecular dynamics simulation in the set are each started with the part of the Tau protein placed beyond hydrogen-bonding distance (such as e.g. at least 4 Å away) of the PHF stack.

The present inventors have found that such molecular dynamics simulation enabled the identification of an intermediate in which the part of the Tau protein has anchored itself on the PHF stack. Such an intermediate may correspond to one where step (i) has occurred.

In embodiments, simulating the conformational changes of the part of the Tau protein from a compact folded state to an aggregated state comprises performing a nudged elastic band molecular dynamics simulation starting from an intermediate in which step (i) has occurred.

According to a further aspect, there is provided a computing system comprising a processor and a memory storing machine-readable instructions that, when executed by the processor, cause the processor to implement the method of any embodiment of the aspects described above.

According to a further aspect, there is provided a computer readable storage medium (or a plurality of computer readable storage media) storing instructions that, when executed by one or more processors, cause the processor(s) to implement the method of any embodiment of the aspects described above.

According to a further aspect, there is provided a computer software product comprising instructions that, when executed by one or more processors, cause the processor(s) to implement the method of any embodiment of the aspects described above.

### FIGURES

FIG. 1. Flowchart illustrating a method for identifying, selecting and/or designing a compound for modulating Tau aggregation according to the present disclosure.

FIG. 2. Flowchart illustrating a method for identifying, selecting and/or designing a compound for modulating Tau aggregation according to the present disclosure.

FIG. 3. Schematic illustration of the steps used to analyse the structure of Tau97 (AspAsn-dGAE) monomers and LMT-bound Tau97 (AspAsn-dGAE) monomers.

FIG. 4. PCA Subspace comparison of 30 molecular trajectories obtained by independent molecular dynamics simulations of Tau97 monomers. A. Hierarchical clustering of PCA subspace of the 30 trajectories. B. Subspace overlap between the 30 molecular trajectories.

FIG. 5. Heatmap of 178 pairwise RMSD measurements between Tau97 protein conformations derived from the MD sampling (ligand free) and PCA analysis of FIGS. 3-4.

FIG. 6. PCA Subspace comparison of 22 molecular trajectories obtained by cycles of molecular dynamics simulations of LMT-bound Tau97 complexes followed by docking rescoring to identify tightly bound complexes.

FIG. 7. PCA subspace comparison between the 22 molecular trajectories of FIG. 6 and the 30 molecular trajectories of FIG. 4.

FIG. 8. LMT-Tau97 pose found to stabilise the monomer Tau97 protein. A. Cartoon representation of Tau97 bound to LMT (sticks representation), the bottom left is the figure rotated by 90 degrees, and also showing the residues within the binding site. Top right is the LMT binding site. Bottom right is a space filling and surface representation of LMT-bound Tau97. The colours correspond to the colours in the primary sequence indicated at the top. B. Plot of the RSMD (root mean square deviation) of the residues surrounding LMT, D₃₁₄-S₃₁₆, E₃₄₂-K₃₅₃, and K₃₇₀-H₃₇₄. Line plot shows the RSMD from the first frame (top curve) and the RSMD from each preceding frame (bottom curve), plotted as the mean for every 200 ps of simulation time. Error bars indicate the standard deviation for every 200 ps of simulation.

FIG. 9. A-B. Cartoon representations of PHF, residues 306-378 from PDB id 5O3L. C. LMT-Tau97 pose of FIG. 8 with Van der Waals surface coloured by electrostatics, blue is electropositive, and red is electronegative. This figure shows that K343 and K347 form an electropositive cap over the ligand binding site and that Phe378 also caps the pocket with a hydrophobic lid. D. Cartoon representation of the LMT-Tau97 pose of FIG. 8 showing residues within 4 Å of LMT within the binding pocket shown as sticks. E. The LMT binding site in greater detail. The colours on A, B, D, E correspond to the colours in the primary sequence indicated at the bottom of the figure.

FIG. 10. Distances between heavy atoms pairs along the final 10 ns of a 50 ns simulation for the protein complex of FIG. 8, and the 30 Tau97 structures of FIG. 4. A. Gln351 to Thr373. B. Gln351 to His374. C. Gln351 to Lys375. D. Arg349 to Thr377. E. Ser316 to Thr373. F. Ser356 to Glu372. F. Glu338 to Val363.

FIG. 11. Cartoon representation of the complex of FIG. 8, highlighting the strong hydrogen bonding features which stabilise the folded state.

FIG. 12. LMT structure and binding pocket. A-C. Chemical structures of MT (A), LMTM (B) and LMT (C). D. Close up view of the cryptic binding pocket of LMT in the Tau97 conformation of FIG. 8, with van der Waals surface coloured by hydrophobicity, with ligand (LMT) pharmacophore features indicated as spheres (hydrophobic features: green spheres, aromatic features: orange spheres, hydrogen-bond donor: magenta spheres).

FIG. 13. Cartoon representation of the approach of a dGAE73 monomer (colour by the standard deviation in position relative to the binding partner in the would-be fully assembled state, spectrum illustrated, from a high of 18.35 Å in red to a low of 0.87 Å in blue) towards a PHF 10 oligomeric stack, cyan cartoon representation. The top picture is from a view horizontal to the PHF stack, and the bottom picture is the same image rotated by 90° towards the reader.

FIG. 14. Snapshots taken from the molecular dynamics simulations of the anchoring stages in PHF assembly. A) The initial stage showing a bound LMT ligand. B) After 1.2 ns of simulation of dGAE73. C) After 41.2 ns of simulation of dGAE73, when anchoring was deemed to be complete. D) At the start of the unfolding of the dGAE73.

FIG. 15. Analysis of the dGAE73 folding pathway during PHF assembly. A-F. Frames 1, 81, 101, 139, 142 and 144, respectively, from the assembly stage of the molecular dynamics simulation—the structure in A is the structure shown in FIG. 14D), from a different angle. G. Scatterplots tracking the progress of PHF formation of a monomeric dGAE73 protein: a point on the plot illustrates at what frame in the MD simulation a residue of the monomeric dGAE73 is close to the final position it would assume in a fully assembled PHF. The Y-axis represents the residue numbering from 308-378, and the X-axis if the frame number from the MD simulation; frames 1-144 (from the beginning to the completion of assembly) on the left and frames 131-144 on the right. The amino acid sequence is shown below, coloured according to the linear epitopes of four antibodies used to probe the assembly process (from N- to C-ter: 319-331, 337-355, 355-367, 367-378)—also indicated by dashed lines in the cartoon representation and along the y axis on the right side of each plot.

FIG. 16. The initial dGAE73 binding before full assembly commences. A. Representation of the top view of one arm of the PHF with the Van der Waals surface coloured according to electrostatic charge (red is electronegative and blue is electropositive). The top left inset is the cartoon representation of the same structure. B. Representation of A, rotated by 90°, the alternating acidic-basic residue wall of the PHF is highlighted.

FIG. 17. dGAE73 proline 332 switch during PHF assembly. A. Psi and omega dihedral angles of Pro332 observed during the molecular dynamics simulation of dGAE73 assembly onto PHF-transitions from trans-cis-trans are indicated on the plot. B-D. Frames 40, 100 and 138 of the simulation shown as stick representation, with Psi, Psi1 and Omega indicated.

FIG. 18. Hydrogen bonding interactions in a PHF. A. The hydrogen bonding interactions observed in a single layer of a dGAE73 monomer in a PHF from residues V306-Ser320 and Gly367-Phe378. B. Cross-β sheet formation within PHFs are formed through multiple hydrogen-bonds along the protein backbone.

FIG. 19. Graphical representation of analysis of densitometry from dot blots with dGAE antibodies during PHF formation. Binding of the antibodies indicates which parts of the sequence fold first. A. Cartoon representation of folded dGAE showing the region that is bound by each antibody used, and the results of exemplary immunoblots. B. Graphical representation of analysis of densitometry from dot blot for antibodies with linear epitopes 306-359, 319-331, 337-355, 355-367, 367-378, 379-390 and 379-391, for first 8 hours of incubation. C. Graphical representation of analysis of densitometry from dot blots for same antibodies as A, showing the full timeline of incubation (0-80 hours).

FIG. 20. RMSD in Å of the epitopes sequences alone (A) and in the dGAE monomer during the simulated assembly into PHFs (B and C). A. Epitope RMSD using the antibody bound epitope conformation as a point of reference (antibody linear epitopes as indicated). B. RMSD with the final assembled epitope conformation as a point of reference (antibody linear epitopes as indicated). C. RMSD with the starting conformation as a point of reference (antibody linear epitopes as indicated).

FIG. 21. Sandwich ELISA graphs showing the increase in immunoreactivity of core region scAbs to dGAE ‘total’, ‘supernatant’ and ‘pellet’ aggregation inhibition samples prepared in the presence of LMTM. dGAE monomer was included as assay control to indicate the binding profiles of each test scAbs to their corresponding epitopes in non-aggregated samples. (A-C) AB3 scAb, (D-F) AB8, (G-1) AB5 scAb, (J-L) AB1 scAb, (M-O) AB7, (P-R) AB2 scAb. Lack of antibody binding in some dGAE+LMTM aggregate pellet samples corresponds to the absence protein present in this group as confirmed by SDS gel (data not included).

FIG. 22. Protease digestion experiments reveal that dGAE filaments contain a protease resistant core (A-B) and that LMT leads to a loss of protease resistance (C). SDS-PAGE gels are shown for the supernatant (A) and pellet (B) following incubation of fibrils of dGAE in reducing conditions with increasing concentrations of proteinase K or Pronase E. The sequence of dGAE is indicated below panel B, with the sequence of the protease resistant core (band at around 8 kDa, revealed by mass spectrometry to correspond to a protected core region of H299-K370 with a theoretical molecular weight of 7.5 kDa) highlighted. C. SDS-PAGE gel showing that in the presence of DTT, LMT is able to prevent inhibition and the resulting soluble dGAE can be digested.

FIG. 23. Immunogold labelling experiments. A. Immunogold labelling of soluble (left), preformed filaments of dGAE (middle) and soluble capped tau350-362/cappedtau350-362 filaments (right) using an antibody binding the region highlighted in the cartoon representation (358-364) confirm that the recognition sequence of this antibody is exposed in soluble dGAE and buried in PHF. B. Immunogold labelling of dGAE filaments using scAbs targeting the regions highlighted in the cartoon representations show very little labelling and thus reduces further following SDS (to remove the soluble dGAE—framed images).

### DETAILED DESCRIPTION

All residue numbers of the Tau protein sequence and structure in the present disclosure refer to the residues of SEQ ID NO:1, which is the sequence of the four repeat isoform 2N4R of human Tau protein (Uniprot ID P10636-8), or homologous positions in other species or variants thereof. Human Tau isoform 2N4R (Uniprot ID P10636-8) corresponds to amino acids 1-124, 376-394 and 461-758 of full length Tau, Uniprot ID P10636 or P10636-1, provided as SEQ ID NO:2.

SEQ ID NO: 1 (Isoform Tau-F, also known as Tau-4, 2N4R, 441 amino acids):
>sp|P10636-8|TAU_HUMAN Isoform Tau-F of Microtubule-associated protein tau
OS = Homo sapiens OX = 9606 GN = MAPT
MAEPROEFEVMEDHAGTYGLGDRKDQGGYTMHQDQEGDTDAGLKESPLQTPTEDGSEEPG
SETSDAKSTPTAEDVTAPLVDEGAPGKQAAAQPHTEIPEGTTAEEAGIGDTPSLEDEAAG
HVTQARMVSKSKDGTGSDDKKAKGADGKTKIATPRGAAPPGQKGQANATRIPAKTPPAPK
TPPSSGEPPKSGDRSGYSSPGSPGTPGSRSRTPSLPTPPTREPKKVAVVRTPPKSPSSAK
SRLQTAPVPMPDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRVQSKIGSLDNI
THVPGGGNKKIETHKLTFRENAKAKTDHGAEIVYKSPVVSGDTSPRHLSNVSSTGSIDMV
DSPQLATLADEVSASLAKQGL
SEQ ID NO: 2 (Full length human Tau, Isoform PNS-Tau, 758 amino acids);
>sp|P10636|TAU_HUMAN Microtubule-associated protein tau OS = Homo sapiens
OX = 9606 GN = MAPT PE = 1 SV = 5
MAEPRQEFEVMEDHAGTYGLGDRKDQGGYTMHQDQEGDTDAGLKESPLQTPTEDGSEEPG
SETSDAKSTPTAEDVTAPLVDEGAPGKQAAAQPHTEIPEGTTAEEAGIGDTPSLEDEAAG
HVTQEPESGKVVQEGFLREPGPPGLSHQLMSGMPGAPLLPEGPREATRQPSGTGPEDTEG
GRHAPELLKHQLLGDLHQEGPPLKGAGGKERPGSKEEVDEDRDVDESSPQDSPPSKASPA
QDGRPPQTAAREATSIPGFPAEGAIPLPVDFLSKVSTEIPASEPDGPSVGRAKGQDAPLE
FTFHVEITPNVQKEQAHSEEHLGRAAFPGAPGEGPEARGPSLGEDTKEADLPEPSEKQPA
AAPRGKPVSRVPQLKARMVSKSKDGTGSDDKKAKTSTRSSAKTLKNRPCLSPKHPTPGSS
DPLIQPSSPAVCPEPPSSPKYVSSVTSRTGSSGAKEMKLKGADGKTKIATPRGAAPPGQK
GQANATRIPAKTPPAPKTPPSSGEPPKSGDRSGYSSPGSPGTPGSRSRTPSLPTPPTREP
KKVAVVRTPPKSPSSAKSRLQTAPVPMPDLKNVKSKIGSTENLKHQPGGGKVQIINKKLD
LSNVQSKCGSKDNIKHVPGGGSVQIVYKPVDLSKVTSKCGSLGNIHHKPGGGQVEVKSEK
LDFKDRVQSKIGSLDNITHVPGGGNKKIETHKLTFRENAKAKTDHGAEIVYKSPVVSGDT
SPRHLSNVSSTGSIDMVDSPQLATLADEVSASLAKQGL

References to residues Leu315, Phe346, Lys347, Val350, Ile354, Ile371, Thr373, and Phe378, refer to the residues in bold and underline in the sequence of SEQ ID NO:1 reproduced below (or the corresponding residues in any fragment, homologue or variant thereof, including e.g. dGAE, Tau97 or dGAE73 as defined below):

```
                10 20 30 40
MAEPRQEFEV MEDHAGTYGL GDRKDQGGYT MHQDQEGDTD
                50 60 70 80
AGLKESPLQT PTEDGSEEPG SETSDAKSTP TAEDVTAPLV
                90 100 110 120
DEGAPGKQAA AQPHTEIPEG TTAEEAGIGD TPSLEDEAAG
              130 140 150 160
HVTQARMVSK SKDGTGSDDK KAKGADGKTK IATPRGAAPP
              170 180 190 200
GQKGQANATR IPAKTPPAPK TPPSSGEPPK SGDRSGYSSP
              210 220 230 240
GSPGTPGSRS RTPSLPTPPT REPKKVAVVR TPPKSPSSAK
              250 260 270 280
SRLQTAPVPM PDLKNVKSKI GSTENLKHQP GGGKVQIINK
              290 300 310 320
KLDLSNVQSK CGSKDNIKHV PGGGSVQIVY KPVDLSKVIS
              330 340 350 360
KCGSLGNIHH KPGGGQVEVK SEKLDFKDRV QSKIGSLDNI
              370 380 390 400
THVPGGGNKK IETHKLIFRE NAKAKTDHGA EIVYKSPVVS
              410 420 430 440
GDTSPRHLSN VSSTGSIDMV DSPQLATLAD EVSASLAKQG L
```

dGAE refers to the 95 residues fragment of Tau (2N4R) with N-terminus at residue Ile-297 and C-terminus at residue Glu-391, as described in SEQ ID NO: 3, or at homologous positions in other species (the residues mentioned referring to the human or mouse Tau sequence, which are identical in this region). As will be apparent to the skilled person, dGAE95 also corresponds to the fragment of the large peripheral nervous system (PNS) Tau isoform (P10636-1) with N-ter at Ile-614 and C-ter at Glu-708. This sequence may sometimes be referred to simply as “dGAE”.

SEQ ID NO: 3 (dGAE, human/mouse, 95 amino acids):
IKHVPGGGSVQIVYKPVDLSKVTSKCGSLGNIHHKPGGGQVEVKSEKLD
FKDRVQSKIGSLDNITHVPGGGNKKIETHKLTFRENAKAKTDHGAE

dGAE73 refers to the fragment of Tau (2N4R) with N-terminus at residue Val-306 and C-terminus at residue Phe-378, as described in SEQ ID NO: 4, or at homologous positions in other species (the residues mentioned referring to the human or mouse Tau sequence, which are identical in this region). As will be apparent to the skilled person, dGAE73 also corresponds to the fragment of Isoform PNS-Tau (P10636-1) with N-ter at Val-623 and C-ter at Phe-695.

SEQ ID NO: 4 (dGAE73, human/mouse, 73 amino
acids):
VQIVYKPVDLSKVTSKCGSLGNIHHKPGGGQVEVKSEKLDFKDRVQSKI
GSLDNITHVPGGGNKKIETHKLTF

Tau97 refers to the 97 residues fragment of Tau (2N4R) with N-terminus at residue Asp-295 and C-terminus at residue Glu-391, as described in SEQ ID NO: 5, or at homologous positions in other species (the residues mentioned referring to the human or mouse Tau sequence, which are identical in this region). As will be apparent to the skilled person, Tau97 also corresponds to the fragment of the large peripheral nervous system (PNS) Tau isoform (P10636-1) with N-ter at Asp-612 and C-ter at Glu-708. This sequence may also be referred to as “dGAE97” of “AspAsn-dGAE” as it includes the 95 amino acids dGAE and the two preceding amino acids Asp and Asn.

SEQ ID NO: 5 (Tau97, human/mouse, 97 amino acids):
DNIKHVPGGGSVQIVYKPVDLSKVTSKCGSLGNIHHKPGGGQVEVKSEK
LDFKDRVQSKIGSLDNITHVPGGGNKKIETHKLTFRENAKAKTDHGAE

In the context of the present disclosure, polypeptides are considered to be “structurally equivalent” if they are able to fold into conformations that have the same three-dimensional interaction properties. In particular, a polypeptide that folds into at least one conformation that has a binding pocket is structurally equivalent to another polypeptide if the latter folds into at least one conformation that has a binding pocket, where the binding pockets of the two polypeptides can be stabilised by forming molecular interactions with the same ligand at corresponding positions in the ligand and the amino acid sequences of the respective polypeptides. For example, two polypeptides may be structurally equivalent if the primary sequence of the first polypeptide differs from the primary sequence of the second polypeptides such that the two polypeptides have at least one predicted conformation that forms a binding pocket, and the interaction properties of the binding pocket are not materially affected by the difference in primary sequence. This may be the case for example where one or more substitutions are made that do not change the conformation of the binding pocket and that do not affect residues that establish stabilising interactions with the ligand, or do not affect said residues in such a way that the stabilising interaction is no longer present. In particular, a variant of dGAE (or Tau97 or dGAE73) may be considered to be structurally equivalent to dGAE (or Tau97 or dGAE73) if it is able to fold in a conformation that forms a binding pocket that comprises multiple hydrophobic side chains and is able to accommodate LMT in a pose where LMT forms stabilising interactions with the amino acids in the variant equivalent to Lys343 and Thr373 of dGAE (or Tau97 or dGAE73).

Aggregation of the tau protein is a hallmark of diseases referred to as “tauopathies”. Various tauopathy disorders that have been recognized which feature prominent tau pathology in neurons and/or glia and this term has been used in the art for several years. The similarities between these pathological inclusions and the characteristic tau inclusions in diseases such as AD indicate that the structural features are shared and that it is the topographic distribution of the pathology that is responsible for the different clinical phenotypes observed. In particular, cryo-electron microscope structures of aggregated Tau in AD, Frontotemporal dementia (including Pick's disease), chronic traumatic encephalopathy (CTE) and cortico-basal degeneration (CBD) have been previously obtained, and all show common conformational features, indicating that compounds that have the ability to modulate Tau aggregation in e.g. PHFs (as observed in AD), may also modulate aggregation of Tau in other tauopathies. In addition to specific diseases discussed below, those skilled in the art can identify tauopathies by combinations of cognitive or behavioural symptoms, plus additionally through the use of appropriate ligands for aggregated tau as visualised using PET or MRI, such as those described in WO02/075318.

Aspects of the present invention relate to “tauopathies”. As well as Alzheimer's disease (AD), the pathogenesis of neurodegenerative disorders such as Pick's disease and Progressive Supranuclear Palsy (PSP) appears to correlate with an accumulation of pathological truncated tau aggregates in the dentate gyrus and stellate pyramidal cells of the neocortex, respectively. Other dementias include fronto-temporal dementia (FTD); parkinsonism linked to chromosome 17 (FTDP-17); disinhibition-dementia-parkinsonism-amyotrophy complex (DDPAC); pallido-ponto-nigral degeneration (PPND); Guam-ALS syndrome; pallido-nigro-luysian degeneration (PNLD); cortico-basal degeneration (CBD); Dementia with Argyrophilic grains (AgD); Dementia pugilistica (DP) wherein despite different topography, NFTs are similar to those observed in AD (Bouras et al., 1992); Chronic traumatic encephalopathy (CTE), a tauopathy including DP as well as repeated and sports-related concussion (McKee, et al., 2009). Others are discussed in Wischik et al. 2000, for detailed discussion—especially Table 5.1).

Abnormal tau in NFTs is found also in Down's Syndrome (DS) (Flament et al., 1990), and in dementia with Lewy bodies (DLB) (Harrington et al., 1994). Tau-positive NFTs are also found in Postencephalitic parkinsonism (PEP) (Charpiot et al., 1992). Glial tau tangles are observed in Subacute sclerosing panencephalitis (SSPE) (Ikeda et al., 1995). Other tauopathies include Niemann-Pick disease type C (NPC) (Love et al., 1995); Sanfilippo syndrome type B (or mucopolysaccharidosis Ill B, MPS Ill B) (Ohmi, et al., 2009); myotonic dystrophies (DM), DM1 (Sergeant, et al., 2001 and references cited therein) and DM2 (Maurage et al., 2005). Additionally there is a growing consensus in the literature that a tau pathology may also contribute more generally to cognitive deficits and decline, including in mild cognitive impairment (MCI) (see e.g. Braak, et al., 2003, Wischik et al., 2018).

All of these diseases, which are characterized primarily or partially by abnormal tau aggregation, are referred to herein as “tauopathies” or “diseases of tau protein aggregation”. In aspects of the invention relating to tauopathies, preferably the tauopathy is selected from the list consisting of the indications above, i.e., AD, PSP, FTD (including Pick's disease), FTDP-17, DDPAC, PPND, Guam-ALS syndrome, PNLD, and CBD and AgD, DS, SSPE, DP, PEP, DLB, CTE and MCI. In one preferred embodiment the tauopathy is Alzheimer's disease (AD). Without wishing to be bound by theory, the present inventors believe that all structures solved for tauopathies encompass the dGAE region of Tau. As such, findings in relation to stabilising a conformation of dGAE (or Tau97) that is not prone to assembly can reasonably be expected to apply to all tau diseases including but not limited to AD.

Aspects of the present disclosure relate to methods for identifying, selecting and/or designing a compound for modulating Tau aggregation, which make use of computer-implemented molecular modelling means.

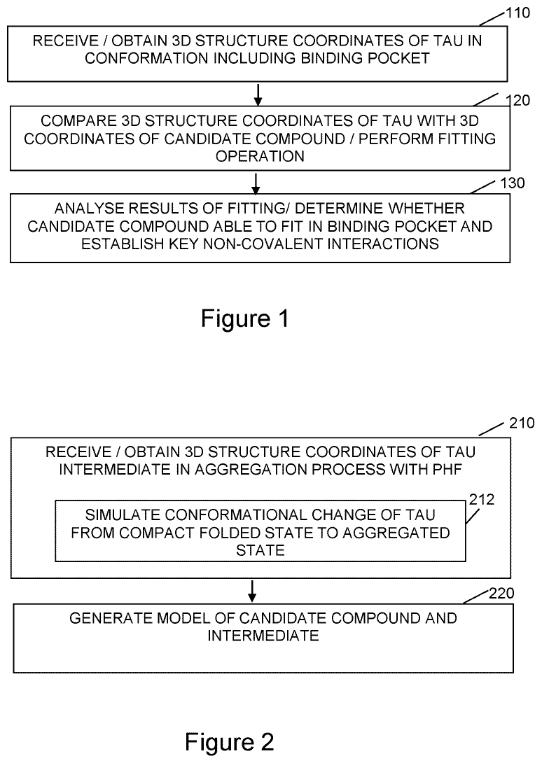

As illustrated in FIG. 1, the method may comprise steps of receiving or obtaining 110 the structure coordinates of a part of the Tau protein as described herein, comparing 120 the three-dimensional structure of a candidate compound with the three-dimensional structure of at least a part of the Tau protein (such as e.g. by performing a fitting operation between a candidate compound and a binding pocket in the Tau protein as described herein), and analysing 130 the results to determine whether the candidate compound is able to bind to the binding pocket. Analysing the results of the fitting operation may comprise determining whether the candidate compound is able fit at least in part within the binding pocket and form non-covalent molecular interactions with one or more of Lys343, and at least one of Leu315, Ser341, Glu342, Lys343, Phe346, Lys347, Val350, Ser352, Ile354, Lys369, Ile371, Glu372, Phe378 and Thr373, of SEQ ID NO:1, or equivalent amino acids in a variant or derivative. Obtaining the structure coordinates of a part of the Tau protein may comprise receiving the structure coordinates of a part of the Tau protein comprising amino acids 315-378 of SEQ ID NO:1 in a PHF stack (such as e.g. from PDB ID: 5O3L), performing molecular dynamics simulations of a part of the Tau protein in the presence of an inhibitor of Tau aggregation as described herein (such as e.g. LMT) and selecting an inhibitor-bound complex conformation using a stability criterion and a binding affinity criterion. Further, performing molecular dynamics simulations may comprise performing a set of independent molecular simulations to obtain a set of molecular trajectories, performing PCA analysis of the molecular trajectories to identify a set of representative conformations, and performing molecular dynamics simulations of a part of the Tau protein in the presence of an inhibitor of Tau aggregation as described herein (such as e.g. LMT) using each of the representative conformations.

As illustrated on FIG. 2, the method may alternatively comprise steps of receiving or obtaining 210 the structure coordinates of a part of the Tau protein, wherein the structure coordinates are those of an intermediate in the aggregation process of the part of the Tau protein with a PHF, and generating 220 a model of a candidate compound and Tau protein. Obtaining 210 the structure coordinates of a part of the Tau protein, wherein the structure coordinates are those of an intermediate in the aggregation process of the part of the Tau protein with a PHF, may comprise simulating 212 the conformational changes of the part of the Tau protein from a compact folded state to an aggregated state by performing a molecular dynamics simulation of the assembly of a Tau protein or a part thereof with a PHF stack. The compact folded state may have been obtained as described above in relation to step 110. In particular, the structure coordinates of a part of the Tau protein in a compact folded state may have been obtained by receiving the structure coordinates of a part of the Tau protein comprising amino acids 315-378 of SEQ ID NO:1 in a PHF stack (such as e.g. from PDB ID: 5O3L), performing molecular dynamics simulations of a part of the Tau protein in the presence of an inhibitor of Tau aggregation as described herein (such as e.g. LMT) and selecting an inhibitor-bound complex conformation using a stability criterion and a binding affinity criterion. Further, performing molecular dynamics simulations may comprise performing a set of independent molecular simulations (e.g. starting from the coordinates in PDB ID: 5O3L or a structure modelled form those coordinates) to obtain a set of molecular trajectories, performing PCA analysis of the molecular trajectories to identify a set of representative conformations, and performing molecular dynamics simulations of a part of the Tau protein in the presence of an inhibitor of Tau aggregation as described herein (such as e.g. LMT) using each of the representative conformations. Alternatively, the structure coordinates of a part of the Tau protein in a compact folded state may have been obtained by performing a set of independent molecular simulations (e.g. starting from the coordinates in PDB ID: 5O3L or a structure modelled form those coordinates) to obtain a set of molecular trajectories, performing PCA analysis of the molecular trajectories to identify a set of representative conformations, and selecting one or more representative conformations as a compact folded state.

Candidate compounds may be selected using in silico methods known in the art. For example, in silico methods via substructure search may be employed. The structure of a known modulator of Tau aggregation, such as e.g. LMT, may be used, and various structural features of the compound (such as e.g. hydrophobic features, H-bond acceptor or donor features, etc.) may be submitted to a program that will search through libraries of chemical compounds for chemicals with substructures that have similar features. The candidate structures may then optionally be reviewed by an expert, for example in order to remove those compounds that are too large. Selected candidates may be submitted to a program that creates structural coordinates for the compounds, such as e.g. AMBER. Docking and scoring of these candidates may be performed using e.g. the MOE software from CCG.

Selected or designed compounds may further be synthesised or obtained and tested for their ability to modulate Tau aggregation, for example in a cell-based aggregation assay as described in Rickard et al., 2017.

Molecular dynamics simulations may be performed as known in the art, for example using the tools available as part of the AMBER software.

### EXAMPLES

### Example 1—Analysis of the Structure of dGAE Monomers

Overview

In this example, the conformational landscape of dGAE monomers in an aqueous environment was studied using explicit water molecular dynamics simulations based on the model from Fitzpatrick et al. (2017)—from PDB identifier 5O3L (https://www.rcsb.org/structure/5O3L). In particular, a slightly extended version of dGAE (Tau97) was used. However, the results herein are believed to be unaffected by the presence of 2 additional N-terminal residues, and equally apply to dGAE. As such, any references to “Tau97” in this example apply equally to dGAE.

Methods

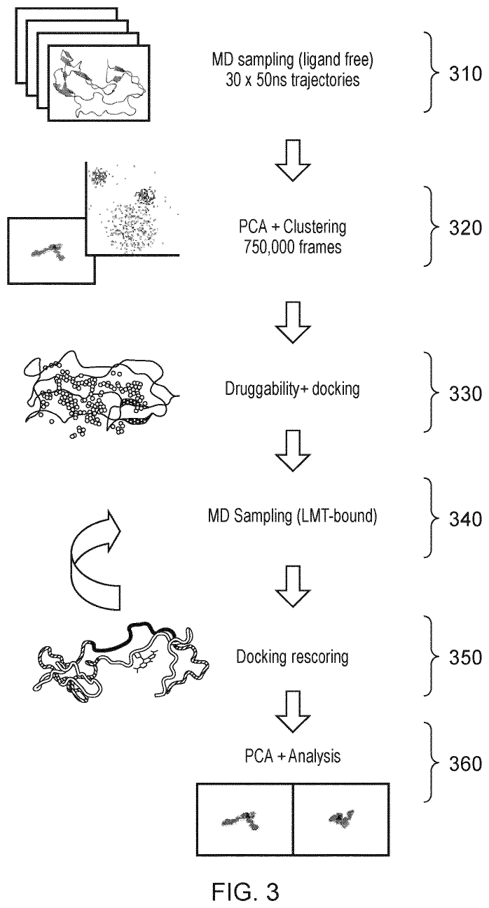

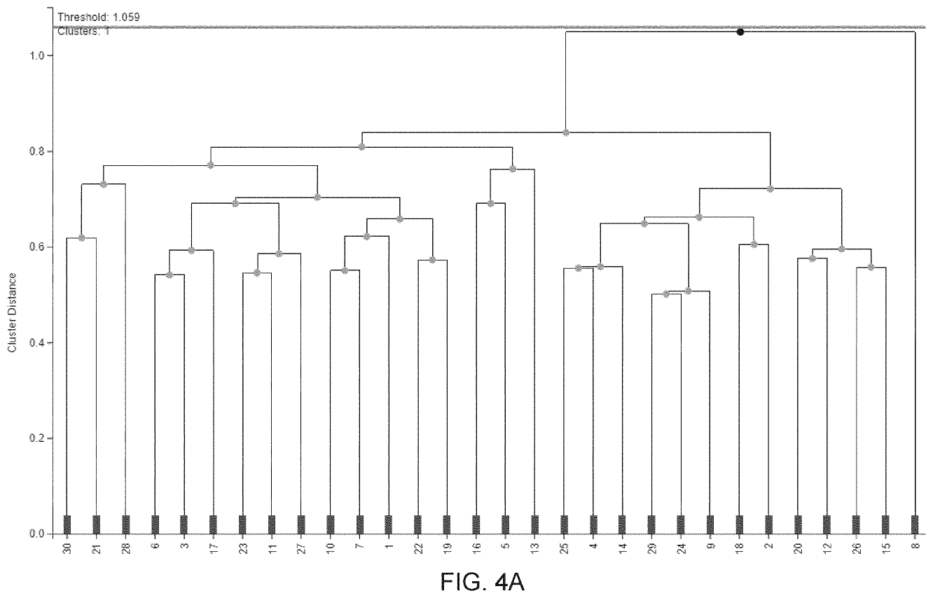

Protein molecular dynamics (MD) simulations (see FIG. 3, Step 310): 3D protein structures of PDB ID: 5O3L (Fitzpatrick et al., 2017) was obtained from the PDB (rcsb.org, Berman et al., 2000). The coordinates of atoms were extracted from the files and only single monomers were used from crystal structures that contained dimers. The sequence utilised in the modelling consisted of Tau97, and refers to the 97-residue fragment of Tau (2N4R) with N-terminus at residue Asp-295 and C-terminus at residue Glu-391 (i.e. comprising the dGAE fragment with N-terminus at residue Asp-297 and C-terminus at residue Glu-391). All the waters were removed from the structures. Protein parameterisation was performed using the MOE 2016.0802 software package (The Molecular Operating Environment, http://www.chemcomp.com).

The structural model was then refined using an energy minimisation process, by means of molecular mechanics using the AMBER force field. Proteins were parameterised in LEaP (a module from the Amber suite of programmes which generates force field files for use with the molecular dynamics packages of Amber) using the AMBER ff14SB force field (Maier et al., 2015). The complexes were neutralised by addition of an appropriate number of chloride counter ions and immersed in a truncated octahedral box of pre-equilibrated TIP3P water molecules (Jorgensen, 1982; Jorgensen et al., 1983). Each water box extended 8 Å away from any solute atom. At each stage, parameter, topology and coordinate files were saved. The cut-off distance for non-bonded interactions was 10 Å. Periodic boundary conditions were applied and electrostatic interactions were represented using the smooth PME method (smooth particle mesh Ewald method; Darden et al., 1998), with constant volume conditions applied. Minimisations were performed using the sander module (MD simulation engine) of AMBER 17 package (Case et al., 2017). The simulation protocol involved initial solvent and ion density equilibration and minimisation. A total of 2,000 minimisation steps were performed; initially 1,000 steps of steepest descent, followed by 1,000 steps of conjugate gradient minimisation. The cut-off distance for non-bonded interactions was 10 Å and a force constant of 500 kcal/mol/Å² was used to restrain the protein. The entire system was then subjected to 2,500 steps of minimisation without restraints.

Following protein refinement using energy minimisation, a 200 ps heating phase with a cut-off distance for non-bonded interactions of 10 Å and a force constant of 10 kcal/mol/Å² was used to restrain the protein, before a 200 ps equilibration run. At this point, 30 independent replicas were generated by randomly assigning different sets of velocities (adjusted to a temperature of 300 K) to the initial coordinates.

For each replica a 60 ns unrestrained trajectory was simulated at 300 K temperature and 1 atm pressure. SHAKE (Ryckaert et al., 1977; a constraint algorithm that can be applied to molecular dynamics simulations to ensure that constraints such as bond length, bond angle and torsion angle constraints are satisfied—in this case the algorithm constrains the vibrational stretching of hydrogen bond lengths, fixing the bond distance to equilibrium value) was applied to all bonds involving hydrogen atoms, allowing an integration step of 2 fs. System coordinates were saved every 100 ps for further analysis.

Analysis of conformational flexibility (FIG. 3, Step 320): Principal Component Analysis (PCA) was used to examine the conformational variability amongst the 30 independent simulations. Analysis of trajectories was done with the cpptraj module of AMBER (Roe and Cheatham, 2013) and a locally modified version of pyPCAzip package (Shkurti et al., 2016). A total of 25,000 frames were extracted from each simulation replica. These frames were evenly taken over the last 50 ns of the trajectories, totally 750,000 frames over the 30 replicas. PCA is commonly used to extract the larger amplitude motions that can be observed in the MD trajectories. A subspace (set of eigenvectors obtained by diagonalization of the covariance matrix, giving a vectorial description of components of the motion) can be derived from a PCA analysis of each trajectory, and these can be compared between trajectories in a simple manner.

Results

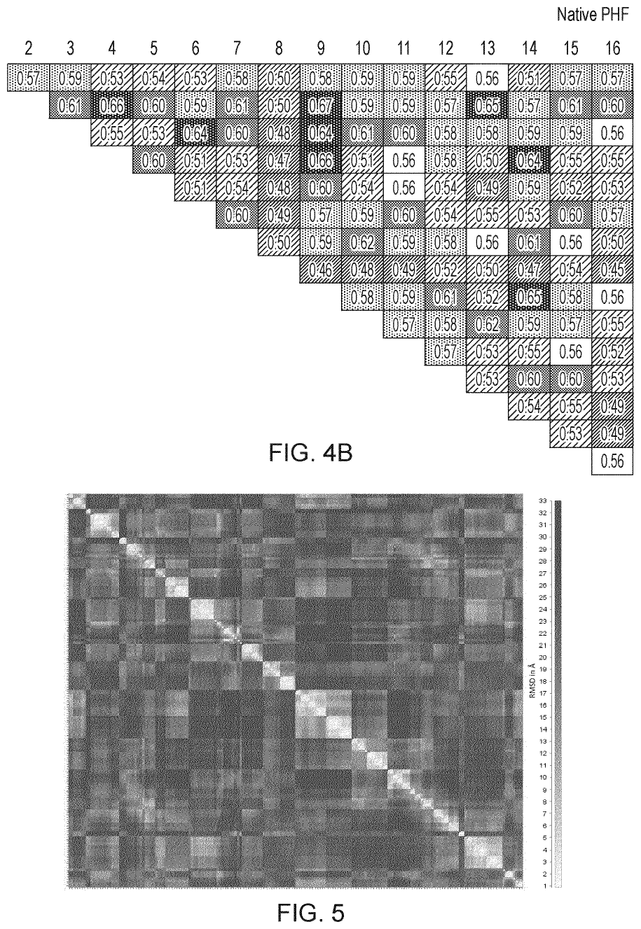

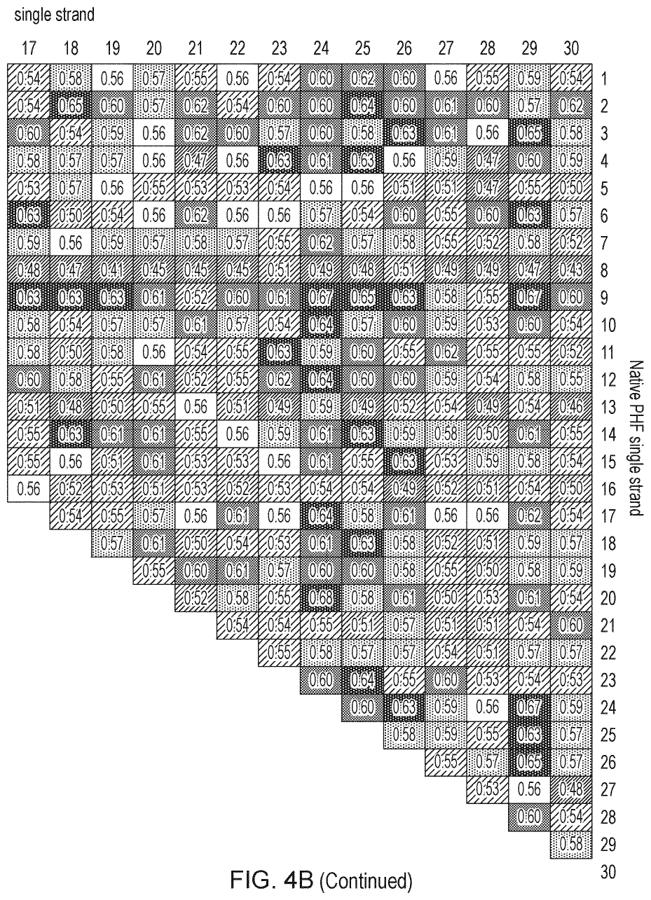

A total of 30 independent 50 ns molecular simulations were set up and analysed using statistical methods to see whether protein folds followed a similar trajectory or whether protein dynamics would fall into clusters. Principal component analysis (PCA) of the molecular trajectories was performed, identifying 19 clusters by clustering in 6 dimensions (see FIG. 4, where FIG. 4A shows the results of hierarchical clustering applied to the PCA subspaces from each trajectory, and FIG. 4B shows the overlap between subspaces for each pair of trajectories). As can be seen on FIG. 4B, the range in subspace overlap was 0.41-0.68. The high value of 0.68 indicates that the dynamics of these proteins follow similar motions. The trajectories represent a collection of possible folds. The inventors were interested in identifying folds which are stable and could be exploited for structure-based drug design purposes. For this purpose, they aimed to make a reasonable wide selection of possible Tau97 conformations to explore, which could be used as a starting point to identify transiently stable cryptic ligand-binding pockets of Tau97 which could be used for structure-based design, using LMT as a molecular probe (as explained in Example 2). The top 20 protein conformations closest in distance (i.e. the 20 closest neighbours) to the centroid of each cluster (i.e. a total of 380 protein conformations) were selected to give a representation of conformational space around the centroid. The root mean square deviation (RMSD) between these conformations was calculated (see FIG. 5) and used to select a diverse set of 178 conformations, for evaluation with molecular docking software.

### Example 2—Analysis of the Structure of LMT-Bound dGAE

Overview

Molecular docking was performed using LMT to identify stable LMT-bound conformations of dGAE. As above, a slightly extended version of dGAE (Tau97) was used. However, the results herein are believed to be unaffected by the presence of 2 additional N-terminal residues, and equally apply to dGAE. As such, any references to “Tau97” in this example apply equally to dGAE.

Methods

Ligand parametrisation: Optimised structures and electrostatic potentials for the ligands were calculated at the MP2/6-311 G* level using General Atomic and Molecular Structure System (GAMESS) (Schmidt et al., 1993; Guest et al., 2005). Non-standard amino acid residues and ligands not described in the parameterisation libraries in LEaP (Schafmeister et al., 1995) were parameterised using the AMBER utility antechamber (Wang et al., 2006) and general AMBER force field (GAFF) (Wang et al., 2004). Ligand parameterisations were checked against the QM calculations by in vacuo MD simulations with sander (Crowley et al., 1997; Pearlman et al., 1995).

Docking and scoring (FIG. 3, Step 330): Molecular docking analysis was performed using the MOE software from CCG. Ligand placement was determined using the Triangular Placement method and 10 poses were trained per query. A total of 65 ligand binding modes were identified with a docking score of <−8.5.

Iterative MD sampling of LMT bound conformations (FIG. 3, Steps 340-350): To assess the strength of the binding interactions identified, the protein ligand complexes were subject to 1 ns of molecular dynamics simulations. The systems were equilibrated as described previously and a 1 ns production run was performed using the AMBER software package. The RMSD of the ligands from their initial binding poses were measured and docking scores re-measured for ligands which remained bound to the protein. Further rounds of 10 ns production runs were performed to identify the most tightly bound ligands, and simulations where the ligand dissociated from the protein were not analysed further. After 40 ns, PCA analysis (FIG. 3, Step 360) using PCAZip was performed on the remaining complexes to assess which complexes were exhibiting similar dynamics. Additionally, PCA analysis was used to analyse and compare the final 25,000 frames from each of these remaining simulation trajectories complexes to those of the final 25,000 frames of the 30 Tau97 simulations of Example 1. After 100 ns a single stable protein conformation was identified which showed consistent tight ligand association and small protein residue RMSD fluctuations (i.e. below about 2.5 Å) as determined by PCA analysis. Protein-ligand interactions and intramolecular interactions within the uniquely identified protein-ligand complex were compared to the 30 Tau97 simulations of Example 1 (final 10 ns of the 50 ns simulations).

Results

In particular, protein docking was performed using LMT against the 178 protein conformations identified in Example 1. This generated 15,090 ligand poses which were refined based on the placement scoring function, with a cutoff of −80 kcal/mol) as determined by visual inspection to 65 candidate binding modes with a docking score <−8.5. The docking scores are based on the assumption that the protein is in a stable conformation, which may not be the case. Therefore, the protein ligand complexes were minimised in explicit solvent and then a 1 ns molecular dynamics run was performed. Complexes that were observed to dissociate from the complex during the 1 ns simulation (based on the RMSD of the ligand during the minimization as well as the ligand binding energy as determined from the docking scoring function) were excluded. This excluded 21 complexes where the ligand was weakly bound. The remaining complexes were subject to a further 10 ns molecular dynamics simulation. Complexes where the LMT ligands had dissociated from Tau97 at that point were eliminated (16 complexes). Further rounds of 10 ns simulation were performed, followed by calculation of the RMSD and ligand binding energy and elimination of the complexes where the protein was too flexible and/or the ligand not tightly bound as determined by visual inspection of the trajectories.

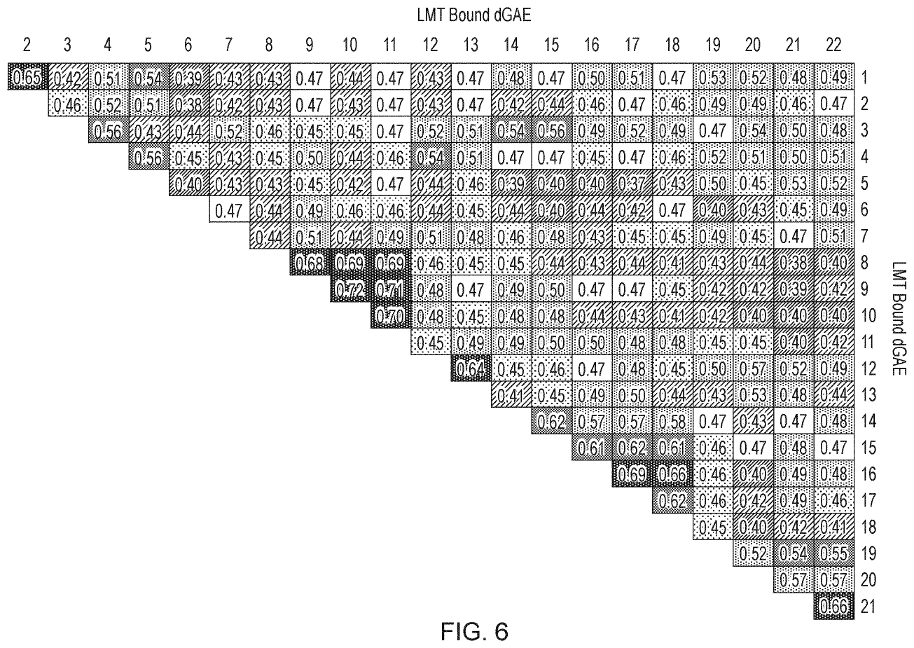

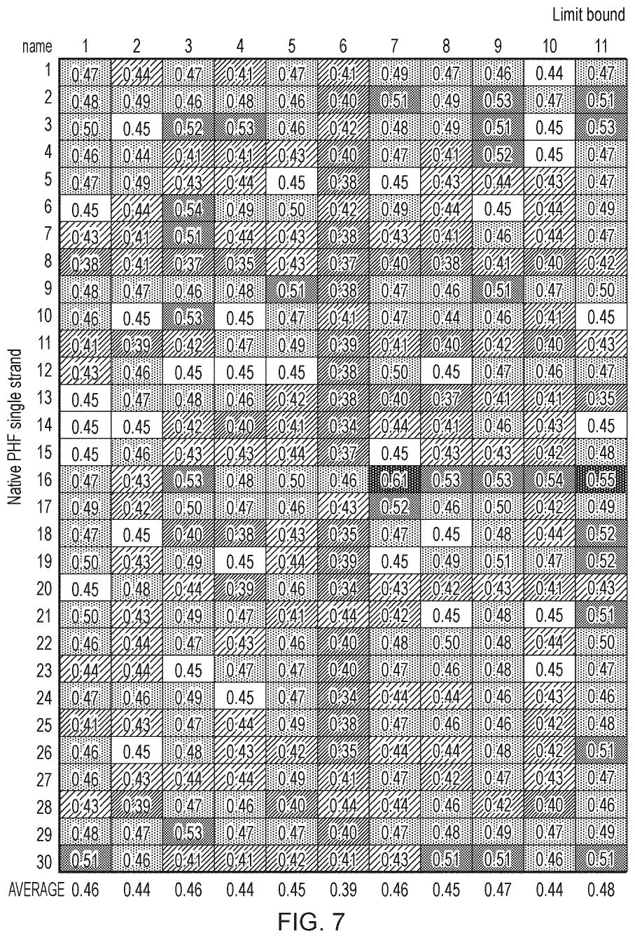

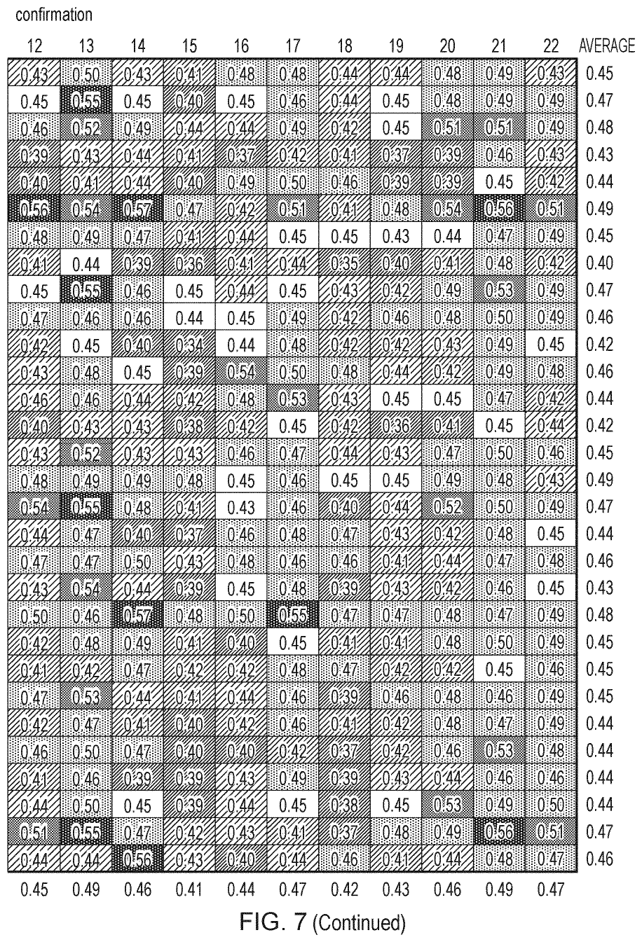

After 40 ns (i.e. a total of 50 ns), 22 complexes remained. PCA analysis of these 22 complexes was performed to assess which complexes were exhibiting similar dynamics. FIG. 6 shows the results of this analysis (pair-wise PCA subspace overlap), with subspaces overlaps ranging between 0.38 and 0.72. An examination of this subspace overlap clearly identified protein complex trajectories which were similar. For example, the range of subspace overlap for complexes 9, 10 and 11 was 0.68 to 0.72, and for complexes 15-18 the subspace overlap was between 0.57 to 0.69. Clustering of these trajectories helped to reduce the number of protein complexes used for further analysis. Comparison of the complexed structures to the trajectory of the 30 Tau97 structures from Example 1 was performed using PCA in an effort to identify unique protein dynamics induced by ligand binding. The results of this analysis are shown on FIG. 7, which shows the PCA Subspace overlap comparison between the Tau97 conformations from Example 1 and the 22 ligand-bound conformations identified through molecular docking and MD sampling. The inventors were interested in whether there was conformational funnelling whereby a large set of different starting protein conformations converged to a smaller set of more similar conformations upon ligand binding, or whether ligand binding was non-specific. Here the range in subspace overlap was 0.34-0.61. The lower value of 0.34 in contrast to the Tau97 subspace overlap comparison (FIG. 4B, range 0.41-0.68) shows that ligand binding allowed the protein to explore new conformation space.

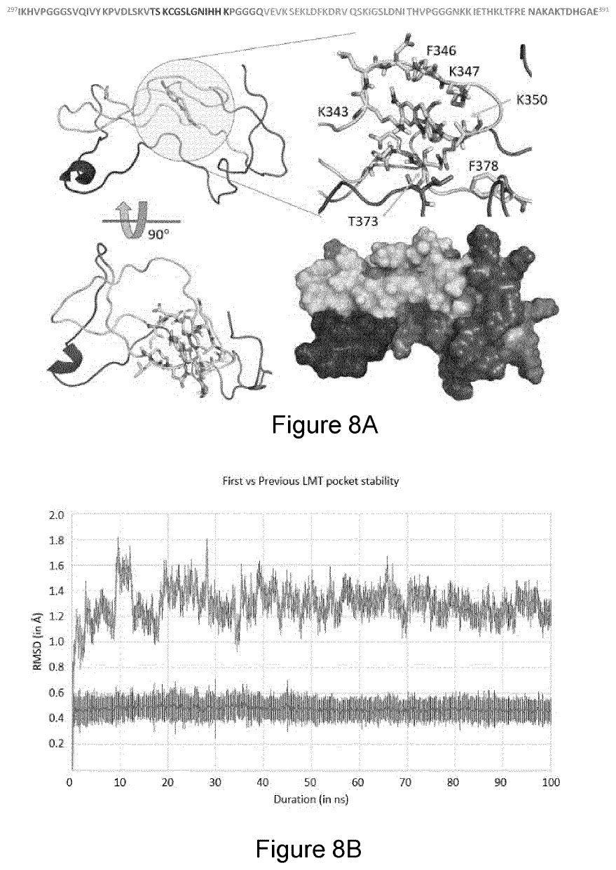

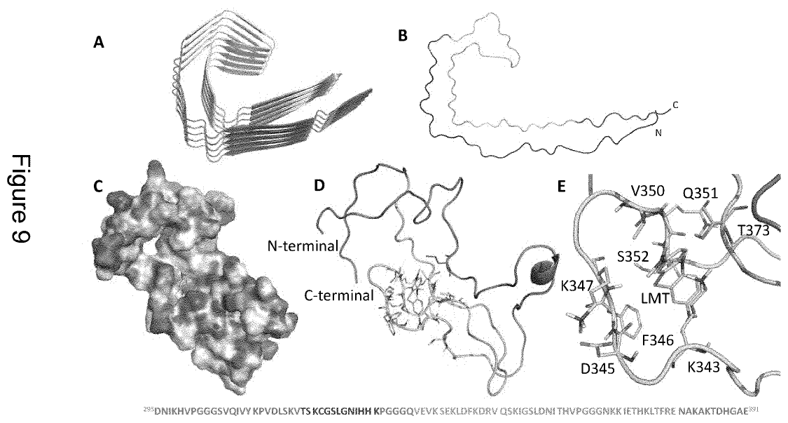

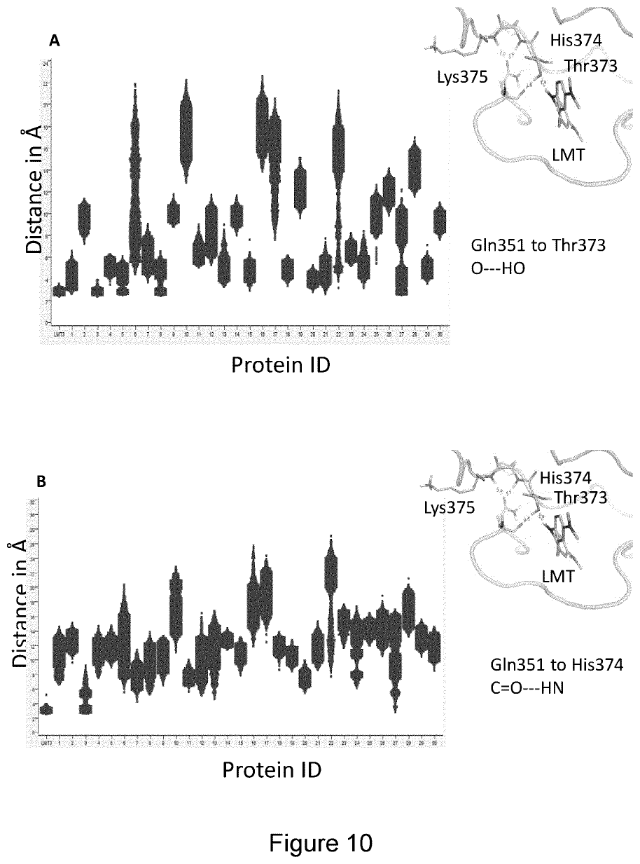

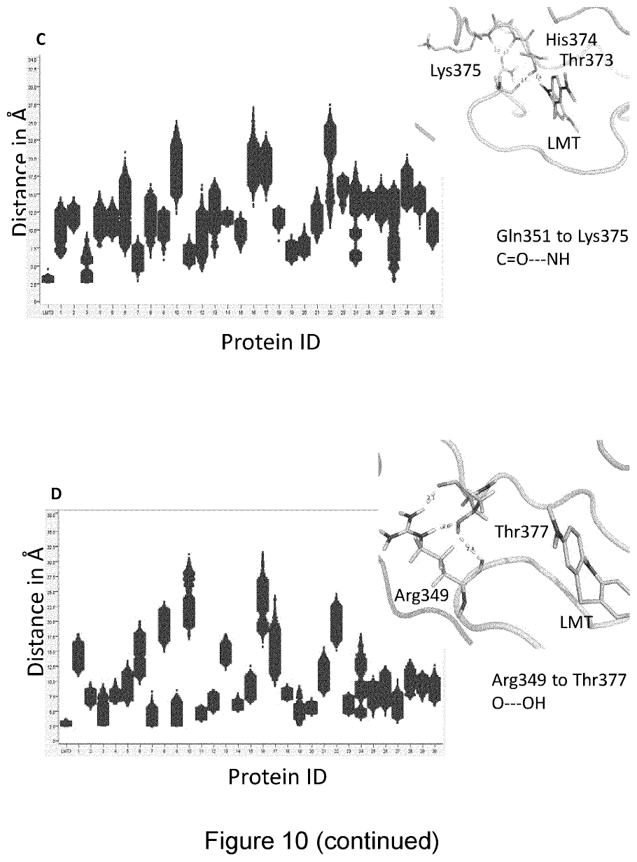

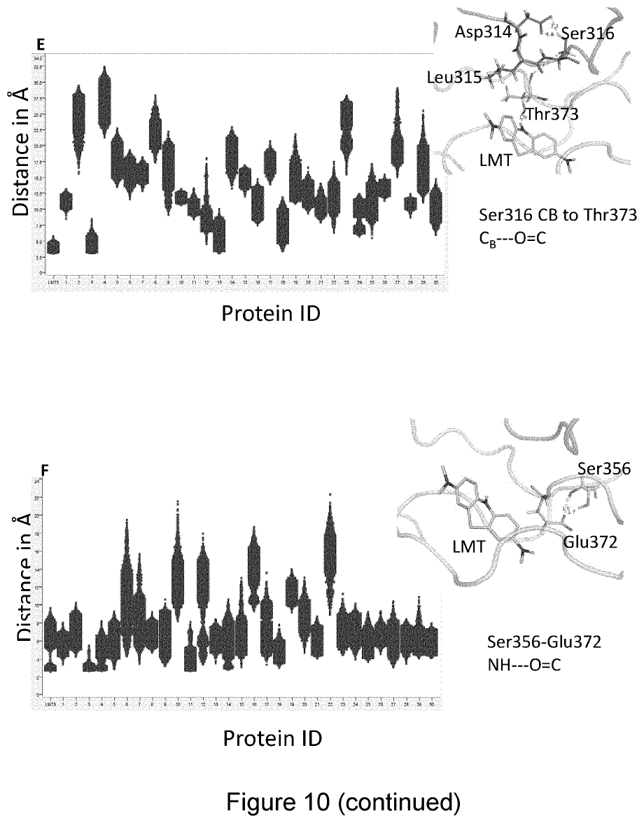

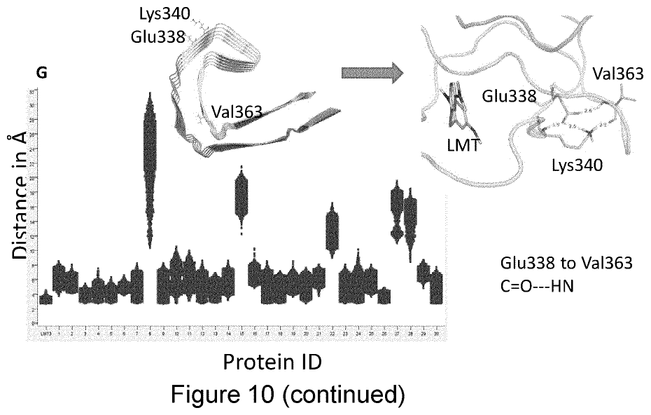

Simulations were continued for a total of 100 ns. A single complex was identified where the ligand remained tightly bound and the protein structure did not vary greatly after a 100 ns of molecular dynamics simulation time, with an average RMSD between frames varying by about 0.5±0.1 Å on average (as shown on FIG. 8). The three-dimensional coordinates of the protein conformation in this complex are provided in Table 1 and further representations of the complex are shown on FIG. 9. This complex corresponds to LMT-bound conformation number 3 in FIGS. 6 and 7. As can be seen on FIG. 7, the protein fold induced upon LMT binding in complex 3 is relatively close to some of the conformations explored with the Tau97 protein as determined by PCA subspace overlap comparison of the molecular dynamics trajectories (PCA subspace overlap ranging between 0.37 and 0.53). Protein-ligand interactions and intramolecular interactions within the uniquely identified protein-ligand complex (LMT-bound conformation number 3) were compared to the 30 Tau97 conformations from the simulations of Example 1 (final 10 ns of the 50 ns simulations), and the results of this are shown on FIG. 10. FIG. 10 shows the distance between pairs of heavy atoms as indicated, in the final 10 ns of the 50 ns simulations of Example 1 (for each of the 30 simulations) and in the final 10 ns of the 100 ns simulation for LMT-bound complex 3 (extreme left on the x axis).

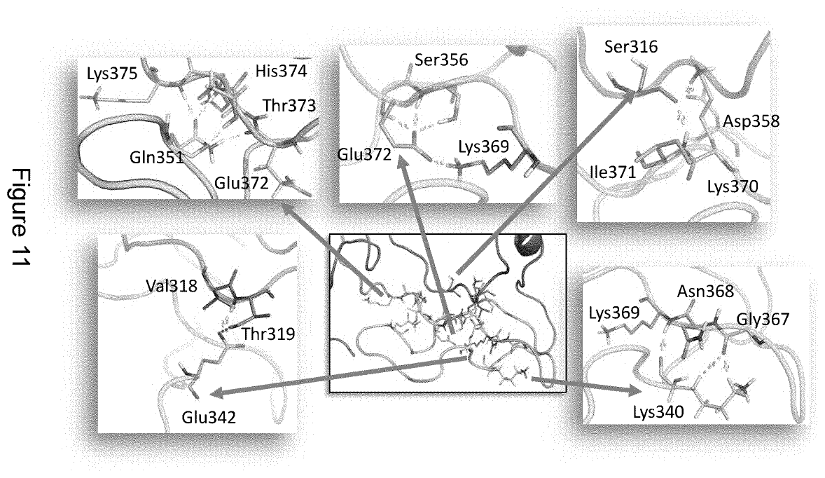

The structure of complex 3 is stabilized by a number of strong hydrogen bonds (see FIG. 11 which shows close-up views of some of these bonds). The Glu342 carboxylic acid makes hydrogen bonds to the backbone NH of Val318 and the sidechain OH of Thr319 (see FIG. 11, bottom left). Each amino acid along the sequence Gly367 to Lys375 form a number of important stabilising hydrogen bonds. Residues Lys369-Thr377 are involved in multiple hydrogen bonds to a tight hairpin formed by residue Ser341-Gln351 (see FIG. 11, top left). Residue Gln351 forms hydrogen bonds through the backbone carbonyl to the Thr373 hydroxyl sidechain (see FIG. 11, top left; distance between Gln351 O and Thr373 OH during the final 10 ns of a 50 ns simulation=2-4 Å, calculated as RMSD between frames of the final 10 ns of the simulation—see FIG. 10A which also shows that the Tau97 conformation 3 of Example 1 has a similar interaction). Residue Gln351 also forms hydrogen bonds through the sidechain carbonyl to the His374 backbone NH (see FIG. 11, top left; distance between Gln351 C═O and His374 NH during the final 10 ns of a 50 ns simulation=2-4 Å—see FIG. 10B which also shows that the dGAE97 conformation 3 of Example 1 has a similar interaction) and to the Lys375 backbone NH (see FIG. 11, top left; distance between Gln351 C═O and Lys375 NH during the final 10 ns of a 50 ns simulation=2.5-5 Å—see FIG. 10C which also shows that the Tau97 conformation 3 of Example 1 has a similar interaction). Arg349 forms three hydrogen bonds to Thr377 (distance between Arg349 O and Thr377 OH during the final 10 ns of a 50 ns simulation=2.5-4 Å, see FIG. 10D). The positively charged sidechain of Arg349 makes hydrogen bonds to the Thr377 hydroxyl sidechain and backbone, and the carbonyl backbone of Arg349 forms a hydrogen bond to the hydroxyl sidechain of Thr377. The carboxylic acid side chain of Glu372 forms multiple hydrogen bonding interactions with Ser356 (see FIG. 11, top middle; distance between Gln372 C═O and Ser356 NH during the final 10 ns of a 50 ns simulation=2-4; 4.5-10 Å, see FIG. 10F) and Lys369. The Ser316 backbone carbonyl makes a hydrogen bond to the backbone NH of Ile371, and the Asp358 sidechain carbonyl makes a hydrogen bond to the Lys370 sidechain amine (see FIG. 11, top right). While some of these interactions are also present in non-LMT bound conformations identified in Example 1, the hydrogen bonding distances are maintained for longer durations for the LMT complex and show considerably less variation than in the conformations identified in example, indicating that LMT has a stabilising influence on these otherwise transient interactions.

The LMT-bound conformation is a compact folded state with no beta sheets (or at least no beta sheets that are intrinsic to the stability of the LMT-bound complex). In other words, the LMT-bound conformation is a compact folded state where any beta sheets that may be present would be in the N or C-termini (which are dynamic sections and could adopt beta sheet conformations at least transiently) and would not be intrinsic to the stability of the LMT-bound conformation of the Tau97/dGAE protein. Without wishing to be bound by theory, the present inventors believe that the absence of beta sheets in this conformation that contributes to inhibition of assembly of the protein into oligomers, such as e.g. PHF. This compact folded state is very different from the structured conformation in PHFs. The compact folded state shows a tight hair pin loop between residues Val337-Gly355. Residues Val363-Gly367 contain a PGGG sequence which is threaded between the PGGG sequence Pro332-Gly335 and a loop formed by the sequence Thr319-Lys331. Further, residues Lys369-Thr377 are sandwiched between residues Asp314-Ser316, with close distances (about 2.5-5.0 Å) between the Ser316 beta-carbon and Thr373 backbone carbonyl (see FIG. 10E, which also shows that this interaction is also observed in Tau97 conformations 3 and 13 from Example 1). This is also the case in some of the conformations identified in example 1, but again the variance and distance between these atoms is smaller for the LMT-bound complex, indicating that LMT stabilises these interactions.

Comparing the starting conformation in Example 1 (which is based on cryo-EM analysis of paired helical filaments-like structures) to the LMT-bound conformation identified herein reveals that residues Gly355-Gly367 and Asn368-Arg379 are brought together in close proximity in the LMT-bound conformation, from a distance of over 36 Å to within hydrogen bonding distance (see FIG. 10G and FIG. 11, bottom right). This is facilitated by the tight loop Val363-PG-Gly366, which enables the sequence Asn368-Arg379 to become folded back towards Lys343-Ser352 (which form a tight loop sometimes referred to as “hairpin loop”). Glu338 is folded towards Val363 (distance between Glu338 C═O and Val363 NH during the final 10 ns of a 50 ns simulation=2-4 Å, see FIG. 10G), a feature which is seen in many of the 30 simulations in example 1, suggesting that without the cross-β sheet structure within PHFs, a monomeric Tau97 protein will favor a wrapped-up conformation. The folding of the protein from a relatively extended conformation in the PHF-like state (starting point of the simulations in Example 1, see FIG. 9A) to the stabilised LMT-bound conformation identified herein reduces the total water accessible surface area over 20%, from about 10223 Å² to about 8014 Å². This results in a reduction in both polar (22%) and hydrophobic (21%) surface areas, indicating that a driving force in forming the compact LMT-bound conformation is the formation on intra-molecular hydrogen bonds and the burying of lipophilic side chains.

TABLE 1
Three-dimensional coordinates of Tau97 conformation with
binding pocket. In this table, occupancy = 1 and
temp factor = 0 for all atoms.

| | Atom | Residue | Residue | | | | |
|---|---|---|---|---|---|---|---|
| c | name | name | number | X (Å) | Y (Å) | Z (Å) | Element |
| 1 | N | ASP | 295 | −11.922 | −4.691 | 18.009 | N1+ |
| 2 | CA | ASP | 295 | −10.501 | −5.028 | 17.787 | C |
| 3 | CB | ASP | 295 | −9.613 | −3.786 | 17.97 | C |
| 4 | CG | ASP | 295 | −9.828 | −2.75 | 16.863 | C |
| 5 | OD1 | ASP | 295 | −10.766 | −2.921 | 16.049 | O |
| 6 | OD2 | ASP | 295 | −9.098 | −1.733 | 16.852 | O1− |
| 7 | C | ASP | 295 | −10.06 | −6.185 | 18.683 | C |
| 8 | O | ASP | 295 | −10.789 | −6.605 | 19.584 | O |
| 9 | H1 | ASP | 295 | −12.075 | −4.43 | 18.971 | H |
| 10 | H2 | ASP | 295 | −12.516 | −5.483 | 17.792 | H |
| 11 | H3 | ASP | 295 | −12.198 | −3.911 | 17.421 | H |
| 12 | HA | ASP | 295 | −10.38 | −5.361 | 16.756 | H |
| 13 | HB2 | ASP | 295 | −8.563 | −4.079 | 17.961 | H |
| 14 | HB3 | ASP | 295 | −9.83 | −3.335 | 18.939 | H |
| 15 | N | ASN | 296 | −8.872 | −6.715 | 18.399 | N |
| 16 | CA | ASN | 296 | −8.244 | −7.877 | 19.021 | C |
| 17 | CB | ASN | 296 | −8.589 | −9.112 | 18.176 | C |
| 18 | CG | ASN | 296 | −10.045 | −9.523 | 18.222 | C |
| 19 | OD1 | ASN | 296 | −10.49 | −10.152 | 19.17 | O |
| 20 | ND2 | ASN | 296 | −10.817 | −9.209 | 17.212 | N |
| 21 | C | ASN | 296 | −6.711 | −7.728 | 19.066 | C |
| 22 | O | ASN | 296 | −6.133 | −6.91 | 18.35 | O |
| 23 | H | ASN | 296 | −8.36 | −6.316 | 17.614 | H |
| 24 | HA | ASN | 296 | −8.61 | −7.998 | 20.038 | H |
| 25 | HB2 | ASN | 296 | −8.311 | −8.896 | 17.148 | H |
| 26 | HB3 | ASN | 296 | −8.007 | −9.971 | 18.509 | H |
| 27 | HD21 | ASN | 296 | −10.492 | −8.577 | 16.486 | H |
| 28 | HD22 | ASN | 296 | −11.793 | −9.487 | 17.26 | H |
| 29 | N | ILE | 297 | −6.05 | −8.63 | 19.797 | N |
| 30 | CA | ILE | 297 | −4.609 | −8.912 | 19.696 | C |
| 31 | CB | ILE | 297 | −3.801 | −8.109 | 20.748 | C |
| 32 | CG2 | ILE | 297 | −4.238 | −8.425 | 22.191 | C |
| 33 | CG1 | ILE | 297 | −2.282 | −8.342 | 20.574 | C |
| 34 | CD1 | ILE | 297 | −1.393 | −7.351 | 21.331 | C |
| 35 | C | ILE | 297 | −4.366 | −10.426 | 19.778 | C |
| 36 | O | ILE | 297 | −5.035 | −11.127 | 20.546 | O |
| 37 | H | ILE | 297 | −6.591 | −9.271 | 20.358 | H |
| 38 | HA | ILE | 297 | −4.274 | −8.588 | 18.709 | H |
| 39 | HB | ILE | 297 | −3.999 | −7.052 | 20.562 | H |
| 40 | HG12 | ILE | 297 | −2.023 | −9.353 | 20.892 | H |
| 41 | HG13 | ILE | 297 | −2.032 | −8.245 | 19.522 | H |
| 42 | HG21 | ILE | 297 | −5.313 | −8.288 | 22.303 | H |
| 43 | HG22 | ILE | 297 | −3.983 | −9.452 | 22.453 | H |
| 44 | HG23 | ILE | 297 | −3.745 | −7.746 | 22.886 | H |
| 45 | HD11 | ILE | 297 | −1.634 | −6.335 | 21.019 | H |
| 46 | HD12 | ILE | 297 | −1.533 | −7.452 | 22.407 | H |
| 47 | HD13 | ILE | 297 | −0.348 | −7.552 | 21.095 | H |
| 48 | N | LYS | 298 | −3.421 | −10.939 | 18.975 | N |
| 49 | CA | LYS | 298 | −2.97 | −12.348 | 18.974 | C |
| 50 | CB | LYS | 298 | −4.038 | −13.236 | 18.298 | C |
| 51 | CG | LYS | 298 | −4.345 | −12.894 | 16.83 | C |
| 52 | CD | LYS | 298 | −5.377 | −13.893 | 16.29 | C |
| 53 | CE | LYS | 298 | −5.71 | −13.638 | 14.818 | C |
| 54 | NZ | LYS | 298 | −6.599 | −14.701 | 14.304 | N1+ |
| 55 | C | LYS | 298 | −1.591 | −12.506 | 18.313 | C |
| 56 | O | LYS | 298 | −0.992 | −11.523 | 17.879 | O |
| 57 | H | LYS | 298 | −2.95 | −10.297 | 18.338 | H |
| 58 | HA | LYS | 298 | −2.87 | −12.679 | 20.009 | H |
| 59 | HB2 | LYS | 298 | −4.965 | −13.159 | 18.865 | H |
| 60 | HB3 | LYS | 298 | −3.719 | −14.278 | 18.359 | H |
| 61 | HG2 | LYS | 298 | −3.432 | −12.958 | 16.239 | H |
| 62 | HG3 | LYS | 298 | −4.748 | −11.883 | 16.759 | H |
| 63 | HD2 | LYS | 298 | −6.294 | −13.822 | 16.879 | H |
| 64 | HD3 | LYS | 298 | −4.974 | −14.901 | 16.395 | H |
| 65 | HE2 | LYS | 298 | −4.786 | −13.624 | 14.243 | H |
| 66 | HE3 | LYS | 298 | −6.188 | −12.661 | 14.717 | H |
| 67 | HZ1 | LYS | 298 | −7.48 | −14.699 | 14.825 | H |
| 68 | HZ2 | LYS | 298 | −6.843 | −14.561 | 13.329 | H |
| 69 | HZ3 | LYS | 298 | −6.156 | −15.611 | 14.406 | H |
| 70 | N | HIE | 299 | −1.08 | −13.737 | 18.198 | N |
| 71 | CA | HIE | 299 | 0.014 | −14.061 | 17.266 | C |
| 72 | CB | HIE | 299 | 1.001 | −15.067 | 17.882 | C |
| 73 | CG | HIE | 299 | 2.186 | −15.33 | 16.979 | C |
| 74 | ND1 | HIE | 299 | 2.181 | −16.167 | 15.859 | N |
| 75 | CE1 | HIE | 299 | 3.375 | −16.001 | 15.266 | C |
| 76 | NE2 | HIE | 299 | 4.126 | −15.135 | 15.962 | N |
| 77 | CD2 | HIE | 299 | 3.392 | −14.695 | 17.043 | C |
| 78 | C | HIE | 299 | −0.544 | −14.585 | 15.935 | C |
| 79 | O | HIE | 299 | −1.36 | −15.506 | 15.926 | O |
| 80 | H | HIE | 299 | −1.622 | −14.509 | 18.559 | H |
| 81 | HA | HIE | 299 | 0.583 | −13.157 | 17.051 | H |
| 82 | HB2 | HIE | 299 | 1.364 | −14.681 | 18.834 | H |
| 83 | HB3 | HIE | 299 | 0.487 | −16.011 | 18.068 | H |
| 84 | HD2 | HIE | 299 | 3.687 | −13.954 | 17.775 | H |
| 85 | HE2 | HIE | 299 | 5.067 | −14.841 | 15.698 | H |
| 86 | HE1 | HIE | 299 | 3.69 | −16.5 | 14.358 | H |
| 87 | N | VAL | 300 | −0.054 | −14.036 | 14.821 | N |
| 88 | CA | VAL | 300 | −0.168 | −14.589 | 13.457 | C |
| 89 | CB | VAL | 300 | −1.297 | −13.939 | 12.627 | C |
| 90 | CG1 | VAL | 300 | −2.666 | −14.318 | 13.184 | C |
| 91 | CG2 | VAL | 300 | −1.205 | −12.411 | 12.56 | C |
| 92 | C | VAL | 300 | 1.175 | −14.414 | 12.733 | C |
| 93 | O | VAL | 300 | 1.965 | −13.567 | 13.161 | O |
| 94 | H | VAL | 300 | 0.624 | −13.296 | 14.925 | H |
| 95 | HA | VAL | 300 | −0.372 | −15.656 | 13.539 | H |
| 96 | HB | VAL | 300 | −1.243 | −14.323 | 11.609 | H |
| 97 | HG11 | VAL | 300 | −3.446 | −13.951 | 12.516 | H |
| 98 | HG12 | VAL | 300 | −2.743 | −15.402 | 13.257 | H |
| 99 | HG13 | VAL | 300 | −2.789 | −13.887 | 14.175 | H |
| 100 | HG21 | VAL | 300 | −1.382 | −11.989 | 13.546 | H |
| 101 | HG22 | VAL | 300 | −0.225 | −12.101 | 12.197 | H |
| 102 | HG23 | VAL | 300 | −1.961 | −12.03 | 11.873 | H |
| 103 | N | PRO | 301 | 1.483 | −15.161 | 11.659 | N |
| 104 | CD | PRO | 301 | 0.732 | −16.291 | 11.124 | C |
| 105 | CG | PRO | 301 | 1.79 | −17.243 | 10.575 | C |
| 106 | CB | PRO | 301 | 2.843 | −16.275 | 10.044 | C |
| 107 | CA | PRO | 301 | 2.819 | −15.133 | 11.063 | C |
| 108 | C | PRO | 301 | 3.273 | −13.793 | 10.449 | C |
| 109 | O | PRO | 301 | 2.526 | −12.811 | 10.318 | O |
| 110 | HA | PRO | 301 | 3.528 | −15.387 | 11.852 | H |
| 111 | HB2 | PRO | 301 | 2.541 | −15.906 | 9.065 | H |
| 112 | HB3 | PRO | 301 | 3.824 | −16.744 | 9.989 | H |
| 113 | HG2 | PRO | 301 | 1.395 | −17.887 | 9.791 | H |
| 114 | HG3 | PRO | 301 | 2.209 | −17.838 | 11.388 | H |
| 115 | HD2 | PRO | 301 | 0.086 | −15.948 | 10.314 | H |
| 116 | HD3 | PRO | 301 | 0.143 | −16.804 | 11.884 | H |
| 117 | N | GLY | 302 | 4.561 | −13.774 | 10.109 | N |
| 118 | CA | GLY | 302 | 5.28 | −12.647 | 9.524 | C |
| 119 | C | GLY | 302 | 6.091 | −11.828 | 10.529 | C |
| 120 | O | GLY | 302 | 6.22 | −12.193 | 11.698 | O |
| 121 | H | GLY | 302 | 5.11 | −14.59 | 10.351 | H |
| 122 | HA2 | GLY | 302 | 4.582 | −11.983 | 9.017 | H |
| 123 | HA3 | GLY | 302 | 5.964 | −13.039 | 8.778 | H |
| 124 | N | GLY | 303 | 6.675 | −10.745 | 10.029 | N |
| 125 | CA | GLY | 303 | 7.471 | −9.755 | 10.758 | C |
| 126 | C | GLY | 303 | 8.18 | −8.857 | 9.742 | C |
| 127 | O | GLY | 303 | 8.794 | −9.376 | 8.818 | O |
| 128 | H | GLY | 303 | 6.561 | −10.592 | 9.03 | H |
| 129 | HA2 | GLY | 303 | 8.218 | −10.248 | 11.38 | H |
| 130 | HA3 | GLY | 303 | 6.814 | −9.161 | 11.391 | H |
| 131 | N | GLY | 304 | 7.949 | −7.544 | 9.765 | N |
| 132 | CA | GLY | 304 | 8.306 | −6.621 | 8.669 | C |
| 133 | C | GLY | 304 | 7.413 | −6.724 | 7.414 | C |
| 134 | O | GLY | 304 | 7.299 | −5.754 | 6.665 | O |
| 135 | H | GLY | 304 | 7.466 | −7.169 | 10.575 | H |
| 136 | HA2 | GLY | 304 | 9.334 | −6.824 | 8.361 | H |
| 137 | HA3 | GLY | 304 | 8.258 | −5.598 | 9.039 | H |
| 138 | N | SER | 305 | 6.7 | −7.844 | 7.257 | N |
| 139 | CA | SER | 305 | 5.646 | −8.136 | 6.276 | C |
| 140 | CB | SER | 305 | 6.319 | −8.675 | 5.006 | C |
| 141 | OG | SER | 305 | 5.416 | −8.856 | 3.929 | O |
| 142 | C | SER | 305 | 4.644 | −9.155 | 6.87 | C |
| 143 | O | SER | 305 | 4.712 | −9.491 | 8.058 | O |
| 144 | H | SER | 305 | 6.917 | −8.598 | 7.894 | H |
| 145 | HA | SER | 305 | 5.112 | −7.226 | 6.018 | H |
| 146 | HB2 | SER | 305 | 7.089 | −7.971 | 4.694 | H |
| 147 | HB3 | SER | 305 | 6.797 | −9.626 | 5.228 | H |
| 148 | HG | SER | 305 | 5.964 | −9.167 | 3.169 | H |
| 149 | N | VAL | 306 | 3.751 | −9.711 | 6.041 | N |
| 150 | CA | VAL | 306 | 3.028 | −10.977 | 6.321 | C |
| 151 | CB | VAL | 306 | 1.807 | −11.164 | 5.396 | C |
| 152 | CG1 | VAL | 306 | 0.849 | −9.974 | 5.501 | C |
| 153 | CG2 | VAL | 306 | 2.19 | −11.372 | 3.924 | C |
| 154 | C | VAL | 306 | 3.95 | −12.204 | 6.224 | C |
| 155 | O | VAL | 306 | 3.698 | −13.229 | 6.857 | O |
| 156 | H | VAL | 306 | 3.756 | −9.366 | 5.088 | H |
| 157 | HA | VAL | 306 | 2.653 | −10.943 | 7.344 | H |
| 158 | HB | VAL | 306 | 1.264 | −12.049 | 5.726 | H |
| 159 | HG11 | VAL | 306 | 1.307 | −9.069 | 5.104 | H |
| 160 | HG12 | VAL | 306 | −0.053 | −10.192 | 4.931 | H |
| 161 | HG13 | VAL | 306 | 0.57 | −9.818 | 6.544 | H |
| 162 | HG21 | VAL | 306 | 2.828 | −10.562 | 3.58 | H |
| 163 | HG22 | VAL | 306 | 2.714 | −12.32 | 3.801 | H |
| 164 | HG23 | VAL | 306 | 1.291 | −11.408 | 3.307 | H |
| 165 | N | GLN | 307 | 5.058 | −12.063 | 5.493 | N |
| 166 | CA | GLN | 307 | 6.253 | −12.908 | 5.556 | C |
| 167 | CB | GLN | 307 | 6.932 | −12.9 | 4.178 | C |
| 168 | CG | GLN | 307 | 6.122 | −13.615 | 3.091 | C |
| 169 | CD | GLN | 307 | 6.786 | −13.446 | 1.732 | C |
| 170 | OE1 | GLN | 307 | 6.659 | −12.404 | 1.1 | O |
| 171 | NE2 | GLN | 307 | 7.592 | −14.388 | 1.29 | N |
| 172 | C | GLN | 307 | 7.222 | −12.376 | 6.628 | C |
| 173 | O | GLN | 307 | 7.053 | −11.26 | 7.12 | O |
| 174 | H | GLN | 307 | 5.164 | −11.182 | 5.014 | H |
| 175 | HA | GLN | 307 | 5.979 | −13.932 | 5.815 | H |
| 176 | HB2 | GLN | 307 | 7.104 | −11.865 | 3.88 | H |
| 177 | HB3 | GLN | 307 | 7.901 | −13.385 | 4.249 | H |
| 178 | HG2 | GLN | 307 | 6.04 | −14.675 | 3.331 | H |
| 179 | HG3 | GLN | 307 | 5.12 | −13.192 | 3.039 | H |
| 180 | HE21 | GLN | 307 | 7.997 | −14.284 | 0.372 | H |
| 181 | HE22 | GLN | 307 | 7.68 | −15.272 | 1.769 | H |
| 182 | N | ILE | 308 | 8.26 | −13.141 | 6.976 | N |
| 183 | CA | ILE | 308 | 9.336 | −12.678 | 7.867 | C |
| 184 | CB | ILE | 308 | 9.885 | −13.833 | 8.736 | C |
| 185 | CG2 | ILE | 308 | 10.974 | −13.285 | 9.681 | C |
| 186 | CG1 | ILE | 308 | 8.771 | −14.518 | 9.566 | C |
| 187 | CD1 | ILE | 308 | 9.216 | −15.845 | 10.196 | C |
| 188 | C | ILE | 308 | 10.426 | −12.021 | 7.008 | C |
| 189 | O | ILE | 308 | 11.096 | −12.698 | 6.222 | O |
| 190 | H | ILE | 308 | 8.382 | −14.04 | 6.514 | H |
| 191 | HA | ILE | 308 | 8.94 | −11.923 | 8.547 | H |
| 192 | HB | ILE | 308 | 10.331 | −14.579 | 8.076 | H |
| 193 | HG12 | ILE | 308 | 8.431 | −13.842 | 10.352 | H |
| 194 | HG13 | ILE | 308 | 7.918 | −14.747 | 8.929 | H |
| 195 | HG21 | ILE | 308 | 11.419 | −14.094 | 10.258 | H |
| 196 | HG22 | ILE | 308 | 11.776 | −12.814 | 9.115 | H |
| 197 | HG23 | ILE | 308 | 10.541 | −12.551 | 10.362 | H |
| 198 | HD11 | ILE | 308 | 9.545 | −16.53 | 9.415 | H |
| 199 | HD12 | ILE | 308 | 10.031 | −15.692 | 10.901 | H |
| 200 | HD13 | ILE | 308 | 8.378 | −16.289 | 10.732 | H |
| 201 | N | VAL | 309 | 10.609 | −10.709 | 7.128 | N |
| 202 | CA | VAL | 309 | 11.491 | −9.885 | 6.283 | C |
| 203 | CB | VAL | 309 | 10.658 | −8.975 | 5.355 | C |
| 204 | CG1 | VAL | 309 | 11.542 | −8.213 | 4.363 | C |
| 205 | CG2 | VAL | 309 | 9.658 | −9.785 | 4.519 | C |
| 206 | C | VAL | 309 | 12.416 | −9.053 | 7.171 | C |
| 207 | O | VAL | 309 | 11.981 | −8.518 | 8.187 | O |
| 208 | H | VAL | 309 | 10.035 | −10.208 | 7.803 | H |
| 209 | HA | VAL | 309 | 12.106 | −10.53 | 5.657 | H |
| 210 | HB | VAL | 309 | 10.103 | −8.256 | 5.959 | H |
| 211 | HG11 | VAL | 309 | 12.056 | −8.903 | 3.694 | H |
| 212 | HG12 | VAL | 309 | 10.917 | −7.54 | 3.776 | H |
| 213 | HG13 | VAL | 309 | 12.271 | −7.597 | 4.883 | H |
| 214 | HG21 | VAL | 309 | 8.94 | −10.281 | 5.168 | H |
| 215 | HG22 | VAL | 309 | 9.112 | −9.116 | 3.855 | H |
| 216 | HG23 | VAL | 309 | 10.183 | −10.529 | 3.921 | H |
| 217 | N | TYR | 310 | 13.701 | −8.942 | 6.812 | N |
| 218 | CA | TYR | 310 | 14.711 | −8.344 | 7.698 | C |
| 219 | CB | TYR | 310 | 16.129 | −8.759 | 7.249 | C |
| 220 | CG | TYR | 310 | 17.03 | −7.695 | 6.632 | C |
| 221 | CD1 | TYR | 310 | 17.792 | −6.854 | 7.469 | C |
| 222 | CE1 | TYR | 310 | 18.706 | −5.93 | 6.926 | C |
| 223 | CZ | TYR | 310 | 18.863 | −5.846 | 5.527 | C |
| 224 | OH | TYR | 310 | 19.719 | −4.94 | 4.977 | O |
| 225 | CE2 | TYR | 310 | 18.093 | −6.678 | 4.685 | C |
| 226 | CD2 | TYR | 310 | 17.18 | −7.601 | 5.234 | C |
| 227 | C | TYR | 310 | 14.542 | −6.829 | 7.925 | C |
| 228 | O | TYR | 310 | 15.056 | −6.308 | 8.915 | O |
| 229 | H | TYR | 310 | 14.016 | −9.405 | 5.974 | H |
| 230 | HA | TYR | 310 | 14.559 | −8.793 | 8.681 | H |
| 231 | HB2 | TYR | 310 | 16.637 | −9.135 | 8.135 | H |
| 232 | HB3 | TYR | 310 | 16.068 | −9.608 | 6.566 | H |
| 233 | HD1 | TYR | 310 | 17.669 | −6.922 | 8.54 | H |
| 234 | HD2 | TYR | 310 | 16.625 | −8.261 | 4.581 | H |
| 235 | HE1 | TYR | 310 | 19.279 | −5.289 | 7.581 | H |
| 236 | HE2 | TYR | 310 | 18.235 | −6.631 | 3.616 | H |
| 237 | HH | TYR | 310 | 20.251 | −4.466 | 5.644 | H |
| 238 | N | LYS | 311 | 13.767 | −6.142 | 7.071 | N |
| 239 | CA | LYS | 311 | 13.35 | −4.734 | 7.197 | C |
| 240 | CB | LYS | 311 | 14.229 | −3.83 | 6.317 | C |
| 241 | CG | LYS | 311 | 15.709 | −3.835 | 6.737 | C |
| 242 | CD | LYS | 311 | 16.626 | −3.02 | 5.817 | C |
| 243 | CE | LYS | 311 | 16.485 | −3.482 | 4.362 | C |
| 244 | NZ | LYS | 311 | 17.735 | −3.291 | 3.606 | N1+ |
| 245 | C | LYS | 311 | 11.874 | −4.587 | 6.777 | C |
| 246 | O | LYS | 311 | 11.412 | −5.376 | 5.952 | O |
| 247 | H | LYS | 311 | 13.319 | −6.667 | 6.333 | H |
| 248 | HA | LYS | 311 | 13.464 | −4.43 | 8.236 | H |
| 249 | HB2 | LYS | 311 | 14.136 | −4.176 | 5.287 | H |
| 250 | HB3 | LYS | 311 | 13.857 | −2.805 | 6.37 | H |
| 251 | HG2 | LYS | 311 | 15.798 | −3.452 | 7.753 | H |
| 252 | HG3 | LYS | 311 | 16.073 | −4.858 | 6.726 | H |
| 253 | HD2 | LYS | 311 | 16.389 | −1.96 | 5.886 | H |
| 254 | HD3 | LYS | 311 | 17.653 | −3.16 | 6.159 | H |
| 255 | HE2 | LYS | 311 | 16.228 | −4.544 | 4.355 | H |
| 256 | HE3 | LYS | 311 | 15.667 | −2.93 | 3.891 | H |
| 257 | HZ1 | LYS | 311 | 18.058 | −2.324 | 3.612 | H |
| 258 | HZ2 | LYS | 311 | 18.482 | −3.844 | 4.004 | H |
| 259 | HZ3 | LYS | 311 | 17.626 | −3.611 | 2.646 | H |
| 260 | N | PRO | 312 | 11.123 | −3.594 | 7.288 | N |
| 261 | CD | PRO | 312 | 11.476 | −2.722 | 8.396 | C |
| 262 | CG | PRO | 312 | 10.145 | −2.324 | 9.023 | C |
| 263 | CB | PRO | 312 | 9.231 | −2.237 | 7.802 | C |
| 264 | CA | PRO | 312 | 9.722 | −3.384 | 6.915 | C |
| 265 | C | PRO | 312 | 9.519 | −3.079 | 5.421 | C |
| 266 | O | PRO | 312 | 10.208 | −2.224 | 4.857 | O |
| 267 | HA | PRO | 312 | 9.173 | −4.285 | 7.174 | H |
| 268 | HB2 | PRO | 312 | 9.401 | −1.283 | 7.3 | H |
| 269 | HB3 | PRO | 312 | 8.18 | −2.352 | 8.069 | H |
| 270 | HG2 | PRO | 312 | 10.226 | −1.377 | 9.555 | H |
| 271 | HG3 | PRO | 312 | 9.799 | −3.115 | 9.69 | H |
| 272 | HD2 | PRO | 312 | 11.985 | −1.84 | 8.009 | H |
| 273 | HD3 | PRO | 312 | 12.09 | −3.219 | 9.146 | H |
| 274 | N | VAL | 313 | 8.547 | −3.735 | 4.78 | N |
| 275 | CA | VAL | 313 | 8.273 | −3.592 | 3.331 | C |
| 276 | CB | VAL | 313 | 7.469 | −4.786 | 2.771 | C |
| 277 | CG1 | VAL | 313 | 8.261 | −6.087 | 2.936 | C |
| 278 | CG2 | VAL | 313 | 6.08 | −4.948 | 3.402 | C |
| 279 | C | VAL | 313 | 7.614 | −2.254 | 2.966 | C |
| 280 | O | VAL | 313 | 7.025 | −1.581 | 3.819 | O |
| 281 | H | VAL | 313 | 8.033 | −4.441 | 5.297 | H |
| 282 | HA | VAL | 313 | 9.234 | −3.603 | 2.815 | H |
| 283 | HB | VAL | 313 | 7.322 | −4.628 | 1.703 | H |
| 284 | HG11 | VAL | 313 | 8.422 | −6.307 | 3.992 | H |
| 285 | HG12 | VAL | 313 | 7.72 | −6.913 | 2.475 | H |
| 286 | HG13 | VAL | 313 | 9.23 | −5.987 | 2.448 | H |
| 287 | HG21 | VAL | 313 | 6.166 | −5.123 | 4.472 | H |
| 288 | HG22 | VAL | 313 | 5.488 | −4.051 | 3.227 | H |
| 289 | HG23 | VAL | 313 | 5.57 | −5.798 | 2.947 | H |
| 290 | N | ASP | 314 | 7.714 | −1.84 | 1.701 | N |
| 291 | CA | ASP | 314 | 7.188 | −0.56 | 1.197 | C |
| 292 | CB | ASP | 314 | 8.191 | 0.002 | 0.174 | C |
| 293 | CG | ASP | 314 | 7.829 | 1.369 | −0.421 | C |
| 294 | OD1 | ASP | 314 | 8.633 | 1.835 | −1.262 | O |
| 295 | OD2 | ASP | 314 | 6.795 | 1.977 | −0.042 | O1− |
| 296 | C | ASP | 314 | 5.784 | −0.738 | 0.596 | C |
| 297 | O | ASP | 314 | 5.635 | −1.349 | −0.464 | O |
| 298 | H | ASP | 314 | 8.188 | −2.441 | 1.029 | H |
| 299 | HA | ASP | 314 | 7.118 | 0.159 | 2.015 | H |
| 300 | HB2 | ASP | 314 | 9.162 | 0.093 | 0.664 | H |
| 301 | HB3 | ASP | 314 | 8.301 | −0.712 | −0.644 | H |
| 302 | N | LEU | 315 | 4.745 | −0.22 | 1.265 | N |
| 303 | CA | LEU | 315 | 3.345 | −0.335 | 0.821 | C |
| 304 | CB | LEU | 315 | 2.436 | −0.692 | 2.01 | C |
| 305 | CG | LEU | 315 | 2.766 | −2.025 | 2.704 | C |
| 306 | CD1 | LEU | 315 | 1.847 | −2.188 | 3.914 | C |
| 307 | CD2 | LEU | 315 | 2.586 | −3.241 | 1.793 | C |
| 308 | C | LEU | 315 | 2.843 | 0.896 | 0.046 | C |
| 309 | O | LEU | 315 | 1.662 | 0.981 | −0.285 | O |
| 310 | H | LEU | 315 | 4.918 | 0.268 | 2.14 | H |
| 311 | HA | LEU | 315 | 3.274 | −1.153 | 0.104 | H |
| 312 | HB2 | LEU | 315 | 2.492 | 0.114 | 2.741 | H |
| 313 | HB3 | LEU | 315 | 1.407 | −0.742 | 1.656 | H |
| 314 | HG | LEU | 315 | 3.795 | −2.011 | 3.061 | H |
| 315 | HD11 | LEU | 315 | 2.014 | −1.365 | 4.609 | H |
| 316 | HD12 | LEU | 315 | 0.805 | −2.178 | 3.602 | H |
| 317 | HD13 | LEU | 315 | 2.053 | −3.129 | 4.418 | H |
| 318 | HD21 | LEU | 315 | 2.763 | −4.16 | 2.352 | H |
| 319 | HD22 | LEU | 315 | 1.579 | −3.26 | 1.38 | H |
| 320 | HD23 | LEU | 315 | 3.299 | −3.198 | 0.971 | H |
| 321 | N | SER | 316 | 3.739 | 1.806 | −0.348 | N |
| 322 | CA | SER | 316 | 3.428 | 2.94 | −1.235 | C |
| 323 | CB | SER | 316 | 4.459 | 4.05 | −1.006 | C |
| 324 | OG | SER | 316 | 5.71 | 3.792 | −1.628 | O |
| 325 | C | SER | 316 | 3.314 | 2.544 | −2.724 | C |
| 326 | O | SER | 316 | 3.472 | 3.388 | −3.616 | O |
| 327 | H | SER | 316 | 4.711 | 1.672 | −0.083 | H |
| 328 | HA | SER | 316 | 2.457 | 3.34 | −0.938 | H |
| 329 | HB2 | SER | 316 | 4.052 | 4.981 | −1.4 | H |
| 330 | HB3 | SER | 316 | 4.622 | 4.17 | 0.064 | H |
| 331 | HG | SER | 316 | 6.201 | 3.133 | −1.079 | H |
| 332 | N | LYS | 317 | 3.176 | 1.237 | −2.993 | N |
| 333 | CA | LYS | 317 | 3.71 | 0.559 | −4.179 | C |
| 334 | CB | LYS | 317 | 5.204 | 0.311 | −3.889 | C |
| 335 | CG | LYS | 317 | 6.032 | −0.045 | −5.132 | C |
| 336 | CD | LYS | 317 | 7.481 | 0.427 | −4.973 | C |
| 337 | CE | LYS | 317 | 8.244 | −0.347 | −3.895 | C |
| 338 | NZ | LYS | 317 | 9.453 | 0.391 | −3.471 | N1+ |
| 339 | C | LYS | 317 | 2.975 | −0.753 | −4.497 | C |
| 340 | O | LYS | 317 | 2.301 | −1.328 | −3.642 | O |
| 341 | H | LYS | 317 | 2.897 | 0.644 | −2.221 | H |
| 342 | HA | LYS | 317 | 3.614 | 1.224 | −5.039 | H |
| 343 | HB2 | LYS | 317 | 5.617 | 1.226 | −3.459 | H |
| 344 | HB3 | LYS | 317 | 5.31 | −0.477 | −3.141 | H |
| 345 | HG2 | LYS | 317 | 6.006 | −1.122 | −5.304 | H |
| 346 | HG3 | LYS | 317 | 5.607 | 0.454 | −6.003 | H |
| 347 | HD2 | LYS | 317 | 7.996 | 0.304 | −5.927 | H |
| 348 | HD3 | LYS | 317 | 7.475 | 1.49 | −4.727 | H |
| 349 | HE2 | LYS | 317 | 7.602 | −0.517 | −3.026 | H |
| 350 | HE3 | LYS | 317 | 8.525 | −1.32 | −4.307 | H |
| 351 | HZ1 | LYS | 317 | 10.012 | 0.679 | −4.272 | H |
| 352 | HZ2 | LYS | 317 | 9.202 | 1.201 | −2.897 | H |
| 353 | HZ3 | LYS | 317 | 10.025 | −0.214 | −2.885 | H |
| 354 | N | VAL | 318 | 3.174 | −1.258 | −5.716 | N |
| 355 | CA | VAL | 318 | 2.666 | −2.546 | −6.217 | C |
| 356 | CB | VAL | 318 | 1.349 | −2.321 | −6.999 | C |
| 357 | CG1 | VAL | 318 | 1.558 | −1.625 | −8.351 | C |
| 358 | CG2 | VAL | 318 | 0.553 | −3.614 | −7.215 | C |
| 359 | C | VAL | 318 | 3.742 | −3.265 | −7.046 | C |
| 360 | O | VAL | 318 | 4.704 | −2.652 | −7.511 | O |
| 361 | H | VAL | 318 | 3.798 | −0.759 | −6.331 | H |
| 362 | HA | VAL | 318 | 2.438 | −3.184 | −5.362 | H |
| 363 | HB | VAL | 318 | 0.719 | −1.67 | −6.391 | H |
| 364 | HG11 | VAL | 318 | 0.59 | −1.402 | −8.799 | H |
| 365 | HG12 | VAL | 318 | 2.096 | −0.688 | −8.211 | H |
| 366 | HG13 | VAL | 318 | 2.12 | −2.269 | −9.029 | H |
| 367 | HG21 | VAL | 318 | 0.408 | −4.12 | −6.26 | H |
| 368 | HG22 | VAL | 318 | −0.426 | −3.374 | −7.63 | H |
| 369 | HG23 | VAL | 318 | 1.064 | −4.277 | −7.911 | H |
| 370 | N | THR | 319 | 3.611 | −4.582 | −7.19 | N |
| 371 | CA | THR | 319 | 4.419 | −5.435 | −8.079 | C |
| 372 | CB | THR | 319 | 4.325 | −6.9 | −7.611 | C |
| 373 | CG2 | THR | 319 | 5.187 | −7.88 | −8.405 | C |
| 374 | OG1 | THR | 319 | 4.766 | −6.987 | −6.269 | O |
| 375 | C | THR | 319 | 3.975 | −5.297 | −9.543 | C |
| 376 | O | THR | 319 | 2.808 | −5.03 | −9.831 | O |
| 377 | H | THR | 319 | 2.861 | −5.029 | −6.675 | H |
| 378 | HA | THR | 319 | 5.462 | −5.127 | −8.016 | H |
| 379 | HB | THR | 319 | 3.286 | −7.223 | −7.652 | H |
| 380 | HG21 | THR | 319 | 6.234 | −7.578 | −8.378 | H |
| 381 | HG22 | THR | 319 | 5.077 | −8.876 | −7.98 | H |
| 382 | HG23 | THR | 319 | 4.847 | −7.941 | −9.437 | H |
| 383 | HG1 | THR | 319 | 4.001 | −6.699 | −5.718 | H |
| 384 | N | SER | 320 | 4.9 | −5.51 | −10.486 | N |
| 385 | CA | SER | 320 | 4.599 | −5.542 | −11.925 | C |
| 386 | CB | SER | 320 | 5.892 | −5.706 | −12.726 | C |
| 387 | OG | SER | 320 | 5.694 | −5.251 | −14.052 | O |
| 388 | C | SER | 320 | 3.584 | −6.631 | −12.324 | C |
| 389 | O | SER | 320 | 3.328 | −7.587 | −11.588 | O |
| 390 | H | SER | 320 | 5.853 | −5.678 | −10.196 | H |
| 391 | HA | SER | 320 | 4.163 | −4.578 | −12.188 | H |
| 392 | HB2 | SER | 320 | 6.68 | −5.106 | −12.272 | H |
| 393 | HB3 | SER | 320 | 6.197 | −6.753 | −12.725 | H |
| 394 | HG | SER | 320 | 6.467 | −5.545 | −14.576 | H |
| 395 | N | LYS | 321 | 3.003 | −6.472 | −13.517 | N |
| 396 | CA | LYS | 321 | 1.883 | −7.247 | −14.079 | C |
| 397 | CB | LYS | 321 | 0.614 | −6.366 | −14.015 | C |
| 398 | CG | LYS | 321 | 0.143 | −5.985 | −12.59 | C |
| 399 | CD | LYS | 321 | −0.642 | −4.659 | −12.584 | C |
| 400 | CE | LYS | 321 | −1.207 | −4.324 | −11.194 | C |
| 401 | NZ | LYS | 321 | −1.814 | −2.97 | −11.171 | N1+ |
| 402 | C | LYS | 321 | 2.241 | −7.623 | −15.526 | C |
| 403 | O | LYS | 321 | 2.673 | −6.744 | −16.274 | O |
| 404 | H | LYS | 321 | 3.378 | −5.733 | −14.098 | H |
| 405 | HA | LYS | 321 | 1.722 | −8.163 | −13.505 | H |
| 406 | HB2 | LYS | 321 | 0.818 | −5.45 | −14.574 | H |
| 407 | HB3 | LYS | 321 | −0.203 | −6.885 | −14.517 | H |
| 408 | HG2 | LYS | 321 | −0.483 | −6.787 | −12.198 | H |
| 409 | HG3 | LYS | 321 | 0.995 | −5.864 | −11.923 | H |
| 410 | HD2 | LYS | 321 | 0.029 | −3.856 | −12.897 | H |
| 411 | HD3 | LYS | 321 | −1.466 | −4.724 | −13.297 | H |
| 412 | HE2 | LYS | 321 | −1.968 | −5.069 | −10.946 | H |
| 413 | HE3 | LYS | 321 | −0.409 | −4.387 | −10.447 | H |
| 414 | HZ1 | LYS | 321 | −2.484 | −2.845 | −11.926 | H |
| 415 | HZ2 | LYS | 321 | −2.316 | −2.766 | −10.305 | H |
| 416 | HZ3 | LYS | 321 | −1.13 | −2.226 | −11.299 | H |
| 417 | N | CYS | 322 | 2.154 | −8.898 | −15.91 | N |
| 418 | CA | CYS | 322 | 2.797 | −9.386 | −17.141 | C |
| 419 | CB | CYS | 322 | 2.809 | −10.921 | −17.134 | C |
| 420 | SG | CYS | 322 | 3.917 | −11.513 | −15.822 | S |
| 421 | C | CYS | 322 | 2.172 | −8.829 | −18.436 | C |
| 422 | O | CYS | 322 | 0.957 | −8.626 | −18.521 | O |
| 423 | H | CYS | 322 | 1.757 | −9.581 | −15.272 | H |
| 424 | HA | CYS | 322 | 3.836 | −9.05 | −17.132 | H |
| 425 | HB2 | CYS | 322 | 1.797 | −11.295 | −16.971 | H |
| 426 | HB3 | CYS | 322 | 3.168 | −11.293 | −18.096 | H |
| 427 | HG | CYS | 322 | 5.07 | −11.282 | −16.471 | H |
| 428 | N | GLY | 323 | 3.004 | −8.638 | −19.465 | N |
| 429 | CA | GLY | 323 | 2.571 | −8.235 | −20.805 | C |
| 430 | C | GLY | 323 | 1.992 | −6.82 | −20.868 | C |
| 431 | O | GLY | 323 | 2.55 | −5.874 | −20.306 | O |
| 432 | H | GLY | 323 | 3.983 | −8.855 | −19.341 | H |
| 433 | HA2 | GLY | 323 | 3.423 | −8.278 | −21.483 | H |
| 434 | HA3 | GLY | 323 | 1.822 | −8.945 | −21.158 | H |
| 435 | N | SER | 324 | 0.848 | −6.673 | −21.539 | N |
| 436 | CA | SER | 324 | 0.166 | −5.401 | −21.834 | C |
| 437 | CB | SER | 324 | −0.845 | −5.619 | −22.973 | C |
| 438 | OG | SER | 324 | −1.617 | −6.787 | −22.757 | O |
| 439 | C | SER | 324 | −0.456 | −4.69 | −20.615 | C |
| 440 | O | SER | 324 | −1.302 | −3.813 | −20.775 | O |
| 441 | H | SER | 324 | 0.404 | −7.502 | −21.912 | H |
| 442 | HA | SER | 324 | 0.917 | −4.714 | −22.22 | H |
| 443 | HB2 | SER | 324 | −1.497 | −4.751 | −23.076 | H |
| 444 | HB3 | SER | 324 | −0.292 | −5.742 | −23.905 | H |
| 445 | HG | SER | 324 | −2.213 | −6.901 | −23.508 | H |
| 446 | N | LEU | 325 | −0.001 | −5.018 | −19.4 | N |
| 447 | CA | LEU | 325 | −0.416 | −4.421 | −18.127 | C |
| 448 | CB | LEU | 325 | −0.895 | −5.547 | −17.189 | C |
| 449 | CG | LEU | 325 | −2.054 | −6.407 | −17.738 | C |
| 450 | CD1 | LEU | 325 | −2.353 | −7.544 | −16.76 | C |
| 451 | CD2 | LEU | 325 | −3.337 | −5.599 | −17.944 | C |
| 452 | C | LEU | 325 | 0.716 | −3.573 | −17.513 | C |
| 453 | O | LEU | 325 | 0.544 | −2.373 | −17.284 | O |
| 454 | H | LEU | 325 | 0.722 | −5.725 | −19.37 | H |
| 455 | HA | LEU | 325 | −1.253 | −3.745 | −18.3 | H |
| 456 | HB2 | LEU | 325 | −0.05 | −6.207 | −16.99 | H |
| 457 | HB3 | LEU | 325 | −1.207 | −5.1 | −16.244 | H |
| 458 | HG | LEU | 325 | −1.764 | −6.855 | −18.688 | H |
| 459 | HD11 | LEU | 325 | −2.669 | −7.142 | −15.797 | H |
| 460 | HD12 | LEU | 325 | −3.148 | −8.174 | −17.163 | H |
| 461 | HD13 | LEU | 325 | −1.461 | −8.155 | −16.624 | H |
| 462 | HD21 | LEU | 325 | −3.629 | −5.109 | −17.015 | H |
| 463 | HD22 | LEU | 325 | −3.187 | −4.85 | −18.721 | H |
| 464 | HD23 | LEU | 325 | −4.141 | −6.262 | −18.266 | H |
| 465 | N | GLY | 326 | 1.928 | −4.129 | −17.382 | N |
| 466 | CA | GLY | 326 | 3.14 | −3.353 | −17.074 | C |
| 467 | C | GLY | 326 | 3.542 | −2.377 | −18.193 | C |
| 468 | O | GLY | 326 | 4.117 | −1.324 | −17.918 | O |
| 469 | H | GLY | 326 | 2.029 | −5.131 | −17.506 | H |
| 470 | HA2 | GLY | 326 | 2.983 | −2.784 | −16.157 | H |
| 471 | HA3 | GLY | 326 | 3.965 | −4.047 | −16.915 | H |
| 472 | N | ASN | 327 | 3.117 | −2.655 | −19.431 | N |
| 473 | CA | ASN | 327 | 3.278 | −1.797 | −20.612 | C |
| 474 | CB | ASN | 327 | 2.943 | −2.678 | −21.842 | C |
| 475 | CG | ASN | 327 | 3.643 | −2.325 | −23.151 | C |
| 476 | OD1 | ASN | 327 | 4.077 | −3.195 | −23.896 | O |
| 477 | ND2 | ASN | 327 | 3.686 | −1.072 | −23.527 | N |
| 478 | C | ASN | 327 | 2.422 | −0.497 | −20.557 | C |
| 479 | O | ASN | 327 | 2.556 | 0.356 | −21.433 | O |
| 480 | H | ASN | 327 | 2.67 | −3.552 | −19.57 | H |
| 481 | HA | ASN | 327 | 4.326 | −1.493 | −20.669 | H |
| 482 | HB2 | ASN | 327 | 3.222 | −3.71 | −21.633 | H |
| 483 | HB3 | ASN | 327 | 1.868 | −2.646 | −22.014 | H |
| 484 | HD21 | ASN | 327 | 3.285 | −0.355 | −22.932 | H |
| 485 | HD22 | ASN | 327 | 4.027 | −0.855 | −24.449 | H |
| 486 | N | ILE | 328 | 1.526 | −0.346 | −19.571 | N |
| 487 | CA | ILE | 328 | 0.665 | 0.835 | −19.377 | C |
| 488 | CB | ILE | 328 | −0.684 | 0.411 | −18.741 | C |
| 489 | CG2 | ILE | 328 | −1.583 | 1.638 | −18.553 | C |
| 490 | CG1 | ILE | 328 | −1.434 | −0.67 | −19.551 | C |
| 491 | CD1 | ILE | 328 | −2.611 | −1.297 | −18.785 | C |
| 492 | C | ILE | 328 | 1.402 | 1.874 | −18.508 | C |
| 493 | O | ILE | 328 | 1.855 | 1.535 | −17.412 | O |
| 494 | H | ILE | 328 | 1.47 | −1.072 | −18.869 | H |
| 495 | HA | ILE | 328 | 0.455 | 1.283 | −20.348 | H |
| 496 | HB | ILE | 328 | −0.477 | 0.001 | −17.755 | H |
| 497 | HG12 | ILE | 328 | −1.796 | −0.243 | −20.487 | H |
| 498 | HG13 | ILE | 328 | −0.748 | −1.478 | −19.794 | H |
| 499 | HG21 | ILE | 328 | −1.065 | 2.378 | −17.953 | H |
| 500 | HG22 | ILE | 328 | −1.844 | 2.064 | −19.522 | H |
| 501 | HG23 | ILE | 328 | −2.495 | 1.373 | −18.019 | H |
| 502 | HD11 | ILE | 328 | −3.401 | −0.57 | −18.616 | H |
| 503 | HD12 | ILE | 328 | −3.023 | −2.124 | −19.362 | H |
| 504 | HD13 | ILE | 328 | −2.267 | −1.678 | −17.823 | H |
| 505 | N | HIE | 329 | 1.47 | 3.14 | −18.941 | N |
| 506 | CA | HIE | 329 | 2.339 | 4.176 | −18.346 | C |
| 507 | CB | HIE | 329 | 3.167 | 4.847 | −19.451 | C |
| 508 | CG | HIE | 329 | 3.957 | 3.888 | −20.302 | C |
| 509 | ND1 | HIE | 329 | 3.775 | 3.676 | −21.674 | N |
| 510 | CE1 | HIE | 329 | 4.707 | 2.771 | −22.024 | C |
| 511 | NE2 | HIE | 329 | 5.458 | 2.435 | −20.961 | N |
| 512 | CD2 | HIE | 329 | 4.995 | 3.121 | −19.864 | C |
| 513 | C | HIE | 329 | 1.595 | 5.26 | −17.552 | C |
| 514 | O | HIE | 329 | 0.539 | 5.713 | −17.989 | O |
| 515 | H | HIE | 329 | 1.028 | 3.36 | −19.828 | H |
| 516 | HA | HIE | 329 | 3.035 | 3.69 | −17.669 | H |
| 517 | HB2 | HIE | 329 | 2.502 | 5.42 | −20.098 | H |
| 518 | HB3 | HIE | 329 | 3.865 | 5.546 | −18.989 | H |
| 519 | HD2 | HIE | 329 | 5.39 | 3.088 | −18.856 | H |
| 520 | HE2 | HIE | 329 | 6.285 | 1.841 | −20.987 | H |
| 521 | HE1 | HIE | 329 | 4.856 | 2.388 | −23.027 | H |
| 522 | N | HID | 330 | 2.184 | 5.773 | −16.462 | N |
| 523 | CA | HID | 330 | 1.728 | 6.991 | −15.757 | C |
| 524 | CB | HID | 330 | 1.314 | 6.688 | −14.301 | C |
| 525 | CG | HID | 330 | 2.242 | 5.829 | −13.472 | C |
| 526 | ND1 | HID | 330 | 1.843 | 4.969 | −12.472 | N |
| 527 | CE1 | HID | 330 | 2.927 | 4.338 | −11.997 | C |
| 528 | NE2 | HID | 330 | 4.03 | 4.769 | −12.631 | N |
| 529 | CD2 | HID | 330 | 3.607 | 5.733 | −13.556 | C |
| 530 | C | HID | 330 | 2.701 | 8.186 | −15.889 | C |
| 531 | O | HID | 330 | 3.891 | 8.021 | −16.183 | O |
| 532 | H | HID | 330 | 3.054 | 5.349 | −16.156 | H |
| 533 | HA | HID | 330 | 0.814 | 7.333 | −16.245 | H |
| 534 | HB2 | HID | 330 | 1.15 | 7.627 | −13.771 | H |
| 535 | HB3 | HID | 330 | 0.35 | 6.179 | −14.338 | H |
| 536 | HD1 | HID | 330 | 0.903 | 4.879 | −12.09 | H |
| 537 | HD2 | HID | 330 | 4.245 | 6.278 | −14.237 | H |
| 538 | HE1 | HID | 330 | 2.917 | 3.605 | −11.199 | H |
| 539 | N | LYS | 331 | 2.166 | 9.412 | −15.742 | N |
| 540 | CA | LYS | 331 | 2.791 | 10.652 | −16.235 | C |
| 541 | CB | LYS | 331 | 2.088 | 11.007 | −17.559 | C |
| 542 | CG | LYS | 331 | 2.808 | 12.105 | −18.359 | C |
| 543 | CD | LYS | 331 | 2.059 | 12.467 | −19.655 | C |
| 544 | CE | LYS | 331 | 1.763 | 11.275 | −20.581 | C |
| 545 | NZ | LYS | 331 | 2.983 | 10.694 | −21.186 | N1+ |
| 546 | C | LYS | 331 | 2.716 | 11.811 | −15.217 | C |
| 547 | O | LYS | 331 | 1.623 | 12.313 | −14.954 | O |
| 548 | H | LYS | 331 | 1.193 | 9.474 | −15.467 | H |
| 549 | HA | LYS | 331 | 3.836 | 10.457 | −16.473 | H |
| 550 | HB2 | LYS | 331 | 2.053 | 10.104 | −18.169 | H |
| 551 | HB3 | LYS | 331 | 1.061 | 11.319 | −17.356 | H |
| 552 | HG2 | LYS | 331 | 2.891 | 13.005 | −17.748 | H |
| 553 | HG3 | LYS | 331 | 3.815 | 11.77 | −18.605 | H |
| 554 | HD2 | LYS | 331 | 1.105 | 12.919 | −19.38 | H |
| 555 | HD3 | LYS | 331 | 2.632 | 13.219 | −20.2 | H |
| 556 | HE2 | LYS | 331 | 1.224 | 10.508 | −20.021 | H |
| 557 | HE3 | LYS | 331 | 1.104 | 11.614 | −21.384 | H |
| 558 | HZ1 | LYS | 331 | 2.728 | 9.898 | −21.772 | H |
| 559 | HZ2 | LYS | 331 | 3.435 | 11.378 | −21.786 | H |
| 560 | HZ3 | LYS | 331 | 3.654 | 10.384 | −20.488 | H |
| 561 | N | PRO | 332 | 3.851 | 12.296 | −14.681 | N |
| 562 | CD | PRO | 332 | 5.141 | 11.626 | −14.65 | C |
| 563 | CG | PRO | 332 | 5.794 | 12.09 | −13.353 | C |
| 564 | CB | PRO | 332 | 5.308 | 13.534 | −13.244 | C |
| 565 | CA | PRO | 332 | 3.887 | 13.481 | −13.819 | C |
| 566 | C | PRO | 332 | 3.499 | 14.775 | −14.558 | C |
| 567 | O | PRO | 332 | 4.041 | 15.088 | −15.623 | O |
| 568 | HA | PRO | 332 | 3.193 | 13.321 | −12.996 | H |
| 569 | HB2 | PRO | 332 | 5.948 | 14.159 | −13.865 | H |
| 570 | HB3 | PRO | 332 | 5.315 | 13.887 | −12.212 | H |
| 571 | HG2 | PRO | 332 | 6.879 | 12.031 | −13.415 | H |
| 572 | HG3 | PRO | 332 | 5.415 | 11.504 | −12.514 | H |
| 573 | HD2 | PRO | 332 | 5.74 | 11.945 | −15.505 | H |
| 574 | HD3 | PRO | 332 | 5.039 | 10.54 | −14.641 | H |
| 575 | N | GLY | 333 | 2.598 | 15.569 | −13.979 | N |
| 576 | CA | GLY | 333 | 2.113 | 16.819 | −14.57 | C |
| 577 | C | GLY | 333 | 0.866 | 17.366 | −13.873 | C |
| 578 | O | GLY | 333 | 0.479 | 16.88 | −12.815 | O |
| 579 | H | GLY | 333 | 2.229 | 15.314 | −13.065 | H |
| 580 | HA2 | GLY | 333 | 2.897 | 17.574 | −14.517 | H |
| 581 | HA3 | GLY | 333 | 1.872 | 16.653 | −15.621 | H |
| 582 | N | GLY | 334 | 0.222 | 18.351 | −14.502 | N |
| 583 | CA | GLY | 334 | −1.034 | 18.96 | −14.054 | C |
| 584 | C | GLY | 334 | −2.087 | 19.023 | −15.167 | C |
| 585 | O | GLY | 334 | −2.053 | 18.261 | −16.137 | O |
| 586 | H | GLY | 334 | 0.57 | 18.633 | −15.414 | H |
| 587 | HA2 | GLY | 334 | −1.458 | 18.397 | −13.221 | H |
| 588 | HA3 | GLY | 334 | −0.829 | 19.972 | −13.705 | H |
| 589 | N | GLY | 335 | −3.039 | 19.944 | −15.058 | N |
| 590 | CA | GLY | 335 | −4.211 | 20.012 | −15.931 | C |
| 591 | C | GLY | 335 | −5.226 | 18.944 | −15.539 | C |
| 592 | O | GLY | 335 | −5.713 | 18.954 | −14.407 | O |
| 593 | H | GLY | 335 | −3.062 | 20.517 | −14.22 | H |
| 594 | HA2 | GLY | 335 | −4.688 | 20.985 | −15.821 | H |
| 595 | HA3 | GLY | 335 | −3.92 | 19.884 | −16.973 | H |
| 596 | N | GLN | 336 | −5.54 | 18.023 | −16.451 | N |
| 597 | CA | GLN | 336 | −6.463 | 16.915 | −16.181 | C |
| 598 | CB | GLN | 336 | −6.677 | 16.059 | −17.441 | C |
| 599 | CG | GLN | 336 | −7.199 | 16.838 | −18.661 | C |
| 600 | CD | GLN | 336 | −6.099 | 17.578 | −19.42 | C |
| 601 | OE1 | GLN | 336 | −5.088 | 17.006 | −19.817 | O |
| 602 | NE2 | GLN | 336 | −6.222 | 18.869 | −19.621 | N |
| 603 | C | GLN | 336 | −5.948 | 16.042 | −15.022 | C |
| 604 | O | GLN | 336 | −4.773 | 15.678 | −15.006 | O |
| 605 | H | GLN | 336 | −5.092 | 18.063 | −17.353 | H |
| 606 | HA | GLN | 336 | −7.423 | 17.34 | −15.888 | H |
| 607 | HB2 | GLN | 336 | −5.746 | 15.553 | −17.703 | H |
| 608 | HB3 | GLN | 336 | −7.412 | 15.293 | −17.195 | H |
| 609 | HG2 | GLN | 336 | −7.657 | 16.132 | −19.354 | H |
| 610 | HG3 | GLN | 336 | −7.977 | 17.533 | −18.343 | H |
| 611 | HE21 | GLN | 336 | −7.037 | 19.373 | −19.267 | H |
| 612 | HE22 | GLN | 336 | −5.552 | 19.312 | −20.241 | H |
| 613 | N | VAL | 337 | −6.817 | 15.683 | −14.075 | N |
| 614 | CA | VAL | 337 | −6.434 | 15.028 | −12.806 | C |
| 615 | CB | VAL | 337 | −7.162 | 15.7 | −11.622 | C |
| 616 | CG1 | VAL | 337 | −6.864 | 15.029 | −10.275 | C |
| 617 | CG2 | VAL | 337 | −6.737 | 17.171 | −11.508 | C |
| 618 | C | VAL | 337 | −6.684 | 13.518 | −12.851 | C |
| 619 | O | VAL | 337 | −7.755 | 13.071 | −13.262 | O |
| 620 | H | VAL | 337 | −7.786 | 15.961 | −14.193 | H |
| 621 | HA | VAL | 337 | −5.364 | 15.172 | −12.647 | H |
| 622 | HB | VAL | 337 | −8.239 | 15.66 | −11.792 | H |
| 623 | HG11 | VAL | 337 | −7.25 | 14.009 | −10.268 | H |
| 624 | HG12 | VAL | 337 | −5.789 | 15.012 | −10.091 | H |
| 625 | HG13 | VAL | 337 | −7.359 | 15.577 | −9.473 | H |
| 626 | HG21 | VAL | 337 | −5.652 | 17.241 | −11.425 | H |
| 627 | HG22 | VAL | 337 | −7.068 | 17.726 | −12.385 | H |
| 628 | HG23 | VAL | 337 | −7.191 | 17.626 | −10.628 | H |
| 629 | N | GLU | 338 | −5.708 | 12.713 | −12.421 | N |
| 630 | CA | GLU | 338 | −5.815 | 11.248 | −12.427 | C |
| 631 | CB | GLU | 338 | −4.402 | 10.639 | −12.536 | C |
| 632 | CG | GLU | 338 | −4.35 | 9.143 | −12.891 | C |
| 633 | CD | GLU | 338 | −5.285 | 8.774 | −14.049 | C |
| 634 | OE1 | GLU | 338 | −6.426 | 8.347 | −13.747 | O |
| 635 | OE2 | GLU | 338 | −4.956 | 8.958 | −15.242 | O1− |
| 636 | C | GLU | 338 | −6.68 | 10.706 | −11.261 | C |
| 637 | O | GLU | 338 | −6.755 | 11.302 | −10.184 | O |
| 638 | H | GLU | 338 | −4.846 | 13.126 | −12.09 | H |
| 639 | HA | GLU | 338 | −6.336 | 10.98 | −13.342 | H |
| 640 | HB2 | GLU | 338 | −3.86 | 11.177 | −13.316 | H |
| 641 | HB3 | GLU | 338 | −3.869 | 10.798 | −11.597 | H |
| 642 | HG2 | GLU | 338 | −3.322 | 8.873 | −13.14 | H |
| 643 | HG3 | GLU | 338 | −4.637 | 8.569 | −12.007 | H |
| 644 | N | VAL | 339 | −7.378 | 9.589 | −11.499 | N |
| 645 | CA | VAL | 339 | −8.361 | 8.936 | −10.605 | C |
| 646 | CB | VAL | 339 | −9.808 | 9.303 | −11.025 | C |
| 647 | CG1 | VAL | 339 | −10.828 | 8.958 | −9.931 | C |
| 648 | CG2 | VAL | 339 | −10.014 | 10.789 | −11.365 | C |
| 649 | C | VAL | 339 | −8.205 | 7.399 | −10.601 | C |
| 650 | O | VAL | 339 | −8.596 | 6.732 | −9.634 | O |
| 651 | H | VAL | 339 | −7.241 | 9.169 | −12.414 | H |
| 652 | HA | VAL | 339 | −8.197 | 9.286 | −9.585 | H |
| 653 | HB | VAL | 339 | −10.056 | 8.732 | −11.918 | H |
| 654 | HG11 | VAL | 339 | −10.847 | 7.884 | −9.749 | H |
| 655 | HG12 | VAL | 339 | −10.572 | 9.478 | −9.007 | H |
| 656 | HG13 | VAL | 339 | −11.828 | 9.258 | −10.243 | H |
| 657 | HG21 | VAL | 339 | −9.745 | 11.412 | −10.513 | H |
| 658 | HG22 | VAL | 339 | −9.408 | 11.074 | −12.224 | H |
| 659 | HG23 | VAL | 339 | −11.058 | 10.969 | −11.622 | H |
| 660 | N | LYS | 340 | −7.584 | 6.817 | −11.639 | N |
| 661 | CA | LYS | 340 | −7.233 | 5.39 | −11.699 | C |
| 662 | CB | LYS | 340 | −6.742 | 5.002 | −13.101 | C |
| 663 | CG | LYS | 340 | −7.901 | 5.036 | −14.108 | C |
| 664 | CD | LYS | 340 | −7.462 | 4.716 | −15.544 | C |
| 665 | CE | LYS | 340 | −6.448 | 5.709 | −16.126 | C |
| 666 | NZ | LYS | 340 | −6.929 | 7.108 | −16.076 | N1+ |
| 667 | C | LYS | 340 | −6.178 | 5.071 | −10.645 | C |
| 668 | O | LYS | 340 | −5.176 | 5.768 | −10.521 | O |
| 669 | H | LYS | 340 | −7.228 | 7.412 | −12.384 | H |
| 670 | HA | LYS | 340 | −8.12 | 4.799 | −11.476 | H |
| 671 | HB2 | LYS | 340 | −5.943 | 5.679 | −13.406 | H |
| 672 | HB3 | LYS | 340 | −6.342 | 3.988 | −13.074 | H |
| 673 | HG2 | LYS | 340 | −8.648 | 4.3 | −13.806 | H |
| 674 | HG3 | LYS | 340 | −8.373 | 6.017 | −14.083 | H |
| 675 | HD2 | LYS | 340 | −7.025 | 3.716 | −15.563 | H |
| 676 | HD3 | LYS | 340 | −8.347 | 4.703 | −16.183 | H |
| 677 | HE2 | LYS | 340 | −5.502 | 5.629 | −15.581 | H |
| 678 | HE3 | LYS | 340 | −6.249 | 5.435 | −17.163 | H |
| 679 | HZ1 | LYS | 340 | −6.988 | 7.425 | −15.109 | H |
| 680 | HZ2 | LYS | 340 | −6.235 | 7.741 | −16.465 | H |
| 681 | HZ3 | LYS | 340 | −7.846 | 7.243 | −16.489 | H |
| 682 | N | SER | 341 | −6.433 | 4.039 | −9.854 | N |
| 683 | CA | SER | 341 | −5.681 | 3.689 | −8.648 | C |
| 684 | CB | SER | 341 | −6.241 | 4.451 | −7.436 | C |
| 685 | OG | SER | 341 | −7.587 | 4.083 | −7.197 | O |
| 686 | C | SER | 341 | −5.745 | 2.183 | −8.384 | C |
| 687 | O | SER | 341 | −6.702 | 1.508 | −8.766 | O |
| 688 | H | SER | 341 | −7.291 | 3.532 | −10.014 | H |
| 689 | HA | SER | 341 | −4.639 | 3.98 | −8.768 | H |
| 690 | HB2 | SER | 341 | −5.64 | 4.219 | −6.556 | H |
| 691 | HB3 | SER | 341 | −6.19 | 5.525 | −7.62 | H |
| 692 | HG | SER | 341 | −7.743 | 4.101 | −6.234 | H |
| 693 | N | GLU | 342 | −4.774 | 1.669 | −7.636 | N |
| 694 | CA | GLU | 342 | −4.743 | 0.277 | −7.177 | C |
| 695 | CB | GLU | 342 | −3.264 | −0.087 | −6.945 | C |
| 696 | CG | GLU | 342 | −2.957 | −1.587 | −6.936 | C |
| 697 | CD | GLU | 342 | −3.262 | −2.278 | −8.273 | C |
| 698 | OE1 | GLU | 342 | −3.393 | −3.521 | −8.288 | O |
| 699 | OE2 | GLU | 342 | −3.378 | −1.613 | −9.33 | O1− |
| 700 | C | GLU | 342 | −5.625 | 0.045 | −5.927 | C |
| 701 | O | GLU | 342 | −5.78 | −1.092 | −5.466 | O |
| 702 | H | GLU | 342 | −3.959 | 2.244 | −7.423 | H |
| 703 | HA | GLU | 342 | −5.142 | −0.352 | −7.974 | H |
| 704 | HB2 | GLU | 342 | −2.657 | 0.357 | −7.735 | H |
| 705 | HB3 | GLU | 342 | −2.939 | 0.348 | −6 | H |
| 706 | HG2 | GLU | 342 | −1.898 | −1.712 | −6.71 | H |
| 707 | HG3 | GLU | 342 | −3.518 | −2.065 | −6.133 | H |
| 708 | N | LYS | 343 | −6.18 | 1.121 | −5.346 | N |
| 709 | CA | LYS | 343 | −7.065 | 1.127 | −4.17 | C |
| 710 | CB | LYS | 343 | −6.257 | 1.302 | −2.867 | C |
| 711 | CG | LYS | 343 | −5.54 | 0.018 | −2.429 | C |
| 712 | CD | LYS | 343 | −4.065 | −0.084 | −2.845 | C |
| 713 | CE | LYS | 343 | −3.477 | −1.487 | −2.625 | C |
| 714 | NZ | LYS | 343 | −4.315 | −2.552 | −3.234 | N1+ |
| 715 | C | LYS | 343 | −8.132 | 2.222 | −4.254 | C |
| 716 | O | LYS | 343 | −7.818 | 3.373 | −4.567 | O |
| 717 | H | LYS | 343 | −6.012 | 2.009 | −5.798 | H |
| 718 | HA | LYS | 343 | −7.596 | 0.177 | −4.12 | H |
| 719 | HB2 | LYS | 343 | −5.553 | 2.133 | −2.949 | H |
| 720 | HB3 | LYS | 343 | −6.968 | 1.548 | −2.079 | H |
| 721 | HG2 | LYS | 343 | −5.578 | −0.049 | −1.348 | H |
| 722 | HG3 | LYS | 343 | −6.107 | −0.823 | −2.819 | H |
| 723 | HD2 | LYS | 343 | −3.955 | 0.181 | −3.892 | H |
| 724 | HD3 | LYS | 343 | −3.486 | 0.635 | −2.262 | H |
| 725 | HE2 | LYS | 343 | −2.478 | −1.511 | −3.073 | H |
| 726 | HE3 | LYS | 343 | −3.365 | −1.669 | −1.552 | H |
| 727 | HZ1 | LYS | 343 | −3.863 | −3.465 | −3.174 | H |
| 728 | HZ2 | LYS | 343 | −5.198 | −2.636 | −2.739 | H |
| 729 | HZ3 | LYS | 343 | −4.502 | −2.359 | −4.212 | H |
| 730 | N | LEU | 344 | −9.362 | 1.863 | −3.879 | N |
| 731 | CA | LEU | 344 | −10.538 | 2.735 | −3.707 | C |
| 732 | CB | LEU | 344 | −11.49 | 2.547 | −4.907 | C |
| 733 | CG | LEU | 344 | −11.01 | 3.106 | −6.259 | C |
| 734 | CD1 | LEU | 344 | −12.063 | 2.805 | −7.326 | C |
| 735 | CD2 | LEU | 344 | −10.807 | 4.621 | −6.208 | C |
| 736 | C | LEU | 344 | −11.306 | 2.455 | −2.394 | C |
| 737 | O | LEU | 344 | −11.918 | 3.361 | −1.826 | O |
| 738 | H | LEU | 344 | −9.505 | 0.878 | −3.701 | H |
| 739 | HA | LEU | 344 | −10.216 | 3.775 | −3.658 | H |
| 740 | HB2 | LEU | 344 | −11.703 | 1.483 | −5.019 | H |
| 741 | HB3 | LEU | 344 | −12.434 | 3.039 | −4.671 | H |
| 742 | HG | LEU | 344 | −10.074 | 2.625 | −6.546 | H |
| 743 | HD11 | LEU | 344 | −12.215 | 1.729 | −7.403 | H |
| 744 | HD12 | LEU | 344 | −13.008 | 3.279 | −7.063 | H |
| 745 | HD13 | LEU | 344 | −11.734 | 3.181 | −8.294 | H |
| 746 | HD21 | LEU | 344 | −11.704 | 5.1 | −5.816 | H |
| 747 | HD22 | LEU | 344 | −9.953 | 4.864 | −5.577 | H |
| 748 | HD23 | LEU | 344 | −10.605 | 5.006 | −7.206 | H |
| 749 | N | ASP | 345 | −11.217 | 1.234 | −1.861 | N |
| 750 | CA | ASP | 345 | −11.762 | 0.816 | −0.563 | C |
| 751 | CB | ASP | 345 | −12.233 | −0.646 | −0.637 | C |
| 752 | CG | ASP | 345 | −13.553 | −0.8 | −1.383 | C |
| 753 | OD1 | ASP | 345 | −13.532 | −0.95 | −2.629 | O |
| 754 | OD2 | ASP | 345 | −14.61 | −0.727 | −0.708 | O1− |
| 755 | C | ASP | 345 | −10.715 | 0.96 | 0.55 | C |
| 756 | O | ASP | 345 | −9.581 | 0.501 | 0.385 | O |
| 757 | H | ASP | 345 | −10.748 | 0.523 | −2.404 | H |
| 758 | HA | ASP | 345 | −12.619 | 1.442 | −0.312 | H |
| 759 | HB2 | ASP | 345 | −11.465 | −1.259 | −1.111 | H |
| 760 | HB3 | ASP | 345 | −12.373 | −1.019 | 0.38 | H |
| 761 | N | PHE | 346 | −11.111 | 1.521 | 1.696 | N |
| 762 | CA | PHE | 346 | −10.254 | 1.734 | 2.875 | C |
| 763 | CB | PHE | 346 | −10.987 | 2.655 | 3.865 | C |
| 764 | CG | PHE | 346 | −11.454 | 3.988 | 3.306 | C |
| 765 | CD1 | PHE | 346 | −12.829 | 4.29 | 3.246 | C |
| 766 | CE1 | PHE | 346 | −13.258 | 5.551 | 2.796 | C |
| 767 | CZ | PHE | 346 | −12.314 | 6.513 | 2.393 | C |
| 768 | CE2 | PHE | 346 | −10.943 | 6.211 | 2.437 | C |
| 769 | CD2 | PHE | 346 | −10.512 | 4.954 | 2.9 | C |
| 770 | C | PHE | 346 | −9.833 | 0.42 | 3.571 | C |
| 771 | O | PHE | 346 | −10.364 | −0.653 | 3.27 | O |
| 772 | H | PHE | 346 | −12.082 | 1.793 | 1.77 | H |
| 773 | HA | PHE | 346 | −9.34 | 2.238 | 2.557 | H |
| 774 | HB2 | PHE | 346 | −11.845 | 2.116 | 4.268 | H |
| 775 | HB3 | PHE | 346 | −10.322 | 2.869 | 4.702 | H |
| 776 | HD1 | PHE | 346 | −13.564 | 3.569 | 3.577 | H |
| 777 | HD2 | PHE | 346 | −9.454 | 4.743 | 2.973 | H |
| 778 | HE1 | PHE | 346 | −14.315 | 5.784 | 2.784 | H |
| 779 | HE2 | PHE | 346 | −10.218 | 6.958 | 2.142 | H |
| 780 | HZ | PHE | 346 | −12.64 | 7.49 | 2.06 | H |
| 781 | N | LYS | 347 | −8.887 | 0.493 | 4.52 | N |
| 782 | CA | LYS | 347 | −8.403 | −0.654 | 5.318 | C |
| 783 | CB | LYS | 347 | −7.213 | −1.295 | 4.564 | C |
| 784 | CG | LYS | 347 | −6.626 | −2.5 | 5.307 | C |
| 785 | CD | LYS | 347 | −5.581 | −3.31 | 4.541 | C |
| 786 | CE | LYS | 347 | −4.922 | −4.355 | 5.451 | C |
| 787 | NZ | LYS | 347 | −4.123 | −3.751 | 6.546 | N1+ |
| 788 | C | LYS | 347 | −8.091 | −0.268 | 6.78 | C |
| 789 | O | LYS | 347 | −7.999 | 0.914 | 7.103 | O |
| 790 | H | LYS | 347 | −8.502 | 1.408 | 4.734 | H |
| 791 | HA | LYS | 347 | −9.2 | −1.399 | 5.368 | H |
| 792 | HB2 | LYS | 347 | −7.558 | −1.624 | 3.582 | H |
| 793 | HB3 | LYS | 347 | −6.429 | −0.549 | 4.423 | H |
| 794 | HG2 | LYS | 347 | −6.139 | −2.11 | 6.185 | H |
| 795 | HG3 | LYS | 347 | −7.429 | −3.165 | 5.615 | H |
| 796 | HD2 | LYS | 347 | −6.069 | −3.818 | 3.708 | H |
| 797 | HD3 | LYS | 347 | −4.817 | −2.644 | 4.155 | H |
| 798 | HE2 | LYS | 347 | −5.708 | −4.98 | 5.882 | H |
| 799 | HE3 | LYS | 347 | −4.284 | −4.998 | 4.836 | H |
| 800 | HZ1 | LYS | 347 | −4.7 | −3.171 | 7.154 | H |
| 801 | HZ2 | LYS | 347 | −3.738 | −4.483 | 7.146 | H |
| 802 | HZ3 | LYS | 347 | −3.361 | −3.2 | 6.184 | H |
| 803 | N | ASP | 348 | −8.012 | −1.257 | 7.675 | N |
| 804 | CA | ASP | 348 | −7.563 | −1.149 | 9.075 | C |
| 805 | CB | ASP | 348 | −8.406 | −2.123 | 9.916 | C |
| 806 | CG | ASP | 348 | −8.022 | −2.085 | 11.391 | C |
| 807 | OD1 | ASP | 348 | −7.458 | −3.089 | 11.868 | O |
| 808 | OD2 | ASP | 348 | −8.12 | −1.014 | 12.031 | O1− |
| 809 | C | ASP | 348 | −6.036 | −1.389 | 9.263 | C |
| 810 | O | ASP | 348 | −5.369 | −1.997 | 8.417 | O |
| 811 | H | ASP | 348 | −8.244 | −2.187 | 7.348 | H |
| 812 | HA | ASP | 348 | −7.78 | −0.14 | 9.432 | H |
| 813 | HB2 | ASP | 348 | −9.462 | −1.878 | 9.819 | H |
| 814 | HB3 | ASP | 348 | −8.26 | −3.135 | 9.534 | H |
| 815 | N | ARG | 349 | −5.485 | −0.934 | 10.402 | N |
| 816 | CA | ARG | 349 | −4.047 | −0.852 | 10.77 | C |
| 817 | CB | ARG | 349 | −3.384 | −2.236 | 10.953 | C |
| 818 | CG | ARG | 349 | −4 | −3.22 | 11.961 | C |
| 819 | CD | ARG | 349 | −4.173 | −2.685 | 13.389 | C |
| 820 | NE | ARG | 349 | −5.513 | −2.116 | 13.602 | N |
| 821 | CZ | ARG | 349 | −6.101 | −1.835 | 14.749 | C |
| 822 | NH1 | ARG | 349 | −5.488 | −1.884 | 15.897 | N |
| 823 | NH2 | ARG | 349 | −7.355 | −1.502 | 14.731 | N1+ |
| 824 | C | ARG | 349 | −3.189 | 0.016 | 9.838 | C |
| 825 | O | ARG | 349 | −2.579 | 0.975 | 10.31 | O |
| 826 | H | ARG | 349 | −6.142 | −0.531 | 11.064 | H |
| 827 | HA | ARG | 349 | −3.993 | −0.354 | 11.739 | H |
| 828 | HB2 | ARG | 349 | −3.339 | −2.74 | 9.987 | H |
| 829 | HB3 | ARG | 349 | −2.352 | −2.065 | 11.265 | H |
| 830 | HG2 | ARG | 349 | −4.953 | −3.585 | 11.579 | H |
| 831 | HG3 | ARG | 349 | −3.329 | −4.078 | 12.016 | H |
| 832 | HD2 | ARG | 349 | −4.047 | −3.526 | 14.072 | H |
| 833 | HD3 | ARG | 349 | −3.401 | −1.945 | 13.605 | H |
| 834 | HE | ARG | 349 | −6.169 | −2.21 | 12.831 | H |
| 835 | HH11 | ARG | 349 | −5.973 | −1.678 | 16.757 | H |
| 836 | HH12 | ARG | 349 | −4.493 | −2.11 | 15.914 | H |
| 837 | HH21 | ARG | 349 | −7.819 | −1.437 | 13.828 | H |
| 838 | HH22 | ARG | 349 | −7.895 | −1.435 | 15.593 | H |
| 839 | N | VAL | 350 | −3.174 | −0.269 | 8.537 | N |
| 840 | CA | VAL | 350 | −2.395 | 0.478 | 7.537 | C |
| 841 | CB | VAL | 350 | −2.142 | −0.371 | 6.272 | C |
| 842 | CG1 | VAL | 350 | −3.373 | −0.501 | 5.372 | C |
| 843 | CG2 | VAL | 350 | −0.988 | 0.16 | 5.414 | C |
| 844 | C | VAL | 350 | −3.044 | 1.827 | 7.221 | C |
| 845 | O | VAL | 350 | −4.263 | 1.99 | 7.304 | O |
| 846 | H | VAL | 350 | −3.791 | −1.009 | 8.234 | H |
| 847 | HA | VAL | 350 | −1.421 | 0.68 | 7.984 | H |
| 848 | HB | VAL | 350 | −1.865 | −1.375 | 6.597 | H |
| 849 | HG11 | VAL | 350 | −3.155 | −1.196 | 4.562 | H |
| 850 | HG12 | VAL | 350 | −4.224 | −0.85 | 5.951 | H |
| 851 | HG13 | VAL | 350 | −3.626 | 0.463 | 4.928 | H |
| 852 | HG21 | VAL | 350 | −0.762 | −0.561 | 4.628 | H |
| 853 | HG22 | VAL | 350 | −1.261 | 1.101 | 4.938 | H |
| 854 | HG23 | VAL | 350 | −0.096 | 0.302 | 6.023 | H |
| 855 | N | GLN | 351 | −2.225 | 2.79 | 6.802 | N |
| 856 | CA | GLN | 351 | −2.669 | 4.092 | 6.319 | C |
| 857 | CB | GLN | 351 | −1.445 | 5.021 | 6.288 | C |
| 858 | CG | GLN | 351 | −1.74 | 6.437 | 5.768 | C |
| 859 | CD | GLN | 351 | −0.474 | 7.148 | 5.304 | C |
| 860 | OE1 | GLN | 351 | 0.639 | 6.77 | 5.641 | O |
| 861 | NE2 | GLN | 351 | −0.584 | 8.111 | 4.42 | N |
| 862 | C | GLN | 351 | −3.329 | 3.967 | 4.932 | C |
| 863 | O | GLN | 351 | −2.636 | 3.847 | 3.919 | O |
| 864 | H | GLN | 351 | −1.24 | 2.583 | 6.742 | H |
| 865 | HA | GLN | 351 | −3.394 | 4.505 | 7.022 | H |
| 866 | HB2 | GLN | 351 | −1.022 | 5.098 | 7.291 | H |
| 867 | HB3 | GLN | 351 | −0.694 | 4.564 | 5.646 | H |
| 868 | HG2 | GLN | 351 | −2.415 | 6.389 | 4.917 | H |
| 869 | HG3 | GLN | 351 | −2.213 | 7.024 | 6.554 | H |
| 870 | HE21 | GLN | 351 | −1.491 | 8.368 | 4.041 | H |
| 871 | HE22 | GLN | 351 | 0.267 | 8.567 | 4.101 | H |
| 872 | N | SER | 352 | −4.651 | 4.16 | 4.899 | N |
| 873 | CA | SER | 352 | −5.449 | 4.493 | 3.706 | C |
| 874 | CB | SER | 352 | −6.635 | 3.526 | 3.548 | C |
| 875 | OG | SER | 352 | −7.593 | 3.559 | 4.593 | O |
| 876 | C | SER | 352 | −5.84 | 5.986 | 3.644 | C |
| 877 | O | SER | 352 | −6.578 | 6.418 | 2.761 | O |
| 878 | H | SER | 352 | −5.144 | 4.145 | 5.788 | H |
| 879 | HA | SER | 352 | −4.818 | 4.33 | 2.833 | H |
| 880 | HB2 | SER | 352 | −7.15 | 3.761 | 2.618 | H |
| 881 | HB3 | SER | 352 | −6.244 | 2.51 | 3.471 | H |
| 882 | HG | SER | 352 | −7.151 | 3.6 | 5.467 | H |
| 883 | N | LYS | 353 | −5.284 | 6.8 | 4.552 | N |
| 884 | CA | LYS | 353 | −5.328 | 8.274 | 4.58 | C |
| 885 | CB | LYS | 353 | −5.023 | 8.719 | 6.023 | C |
| 886 | CG | LYS | 353 | −5.325 | 10.196 | 6.303 | C |
| 887 | CD | LYS | 353 | −5.05 | 10.529 | 7.774 | C |
| 888 | CE | LYS | 353 | −5.543 | 11.943 | 8.093 | C |
| 889 | NZ | LYS | 353 | −5.4 | 12.238 | 9.536 | N1+ |
| 890 | C | LYS | 353 | −4.343 | 8.874 | 3.56 | C |
| 891 | O | LYS | 353 | −3.263 | 8.31 | 3.385 | O |
| 892 | H | LYS | 353 | −4.689 | 6.352 | 5.229 | H |
| 893 | HA | LYS | 353 | −6.335 | 8.599 | 4.32 | H |
| 894 | HB2 | LYS | 353 | −5.631 | 8.118 | 6.704 | H |
| 895 | HB3 | LYS | 353 | −3.973 | 8.525 | 6.243 | H |
| 896 | HG2 | LYS | 353 | −4.702 | 10.828 | 5.671 | H |
| 897 | HG3 | LYS | 353 | −6.376 | 10.388 | 6.09 | H |
| 898 | HD2 | LYS | 353 | −5.576 | 9.815 | 8.41 | H |
| 899 | HD3 | LYS | 353 | −3.978 | 10.456 | 7.971 | H |
| 900 | HE2 | LYS | 353 | −4.967 | 12.663 | 7.504 | H |
| 901 | HE3 | LYS | 353 | −6.596 | 12.018 | 7.808 | H |
| 902 | HZ1 | LYS | 353 | −4.422 | 12.208 | 9.81 | H |
| 903 | HZ2 | LYS | 353 | −5.776 | 13.148 | 9.767 | H |
| 904 | HZ3 | LYS | 353 | −5.909 | 11.559 | 10.093 | H |
| 905 | N | ILE | 354 | −4.669 | 10.012 | 2.933 | N |
| 906 | CA | ILE | 354 | −3.851 | 10.612 | 1.854 | C |
| 907 | CB | ILE | 354 | −4.49 | 11.857 | 1.198 | C |
| 908 | CG2 | ILE | 354 | −5.875 | 11.535 | 0.615 | C |
| 909 | CG1 | ILE | 354 | −4.473 | 13.148 | 2.056 | C |
| 910 | CD1 | ILE | 354 | −5.382 | 13.198 | 3.288 | C |
| 911 | C | ILE | 354 | −2.397 | 10.916 | 2.255 | C |
| 912 | O | ILE | 354 | −2.127 | 11.249 | 3.414 | O |
| 913 | H | ILE | 354 | −5.559 | 10.441 | 3.152 | H |
| 914 | HA | ILE | 354 | −3.8 | 9.866 | 1.068 | H |
| 915 | HB | ILE | 354 | −3.864 | 12.081 | 0.333 | H |
| 916 | HG12 | ILE | 354 | −3.452 | 13.353 | 2.377 | H |
| 917 | HG13 | ILE | 354 | −4.754 | 13.977 | 1.414 | H |
| 918 | HG21 | ILE | 354 | −6.223 | 12.374 | 0.012 | H |
| 919 | HG22 | ILE | 354 | −5.808 | 10.66 | −0.032 | H |
| 920 | HG23 | ILE | 354 | −6.598 | 11.338 | 1.403 | H |
| 921 | HD11 | ILE | 354 | −6.424 | 13.09 | 2.992 | H |
| 922 | HD12 | ILE | 354 | −5.109 | 12.417 | 3.994 | H |
| 923 | HD13 | ILE | 354 | −5.263 | 14.164 | 3.78 | H |
| 924 | N | GLY | 355 | −1.48 | 10.871 | 1.283 | N |
| 925 | CA | GLY | 355 | −0.044 | 11.093 | 1.498 | C |
| 926 | C | GLY | 355 | 0.813 | 11.379 | 0.253 | C |
| 927 | O | GLY | 355 | 1.931 | 11.867 | 0.409 | O |
| 928 | H | GLY | 355 | −1.791 | 10.604 | 0.35 | H |
| 929 | HA2 | GLY | 355 | 0.087 | 11.931 | 2.183 | H |
| 930 | HA3 | GLY | 355 | 0.367 | 10.205 | 1.976 | H |
| 931 | N | SER | 356 | 0.326 | 11.166 | −0.98 | N |
| 932 | CA | SER | 356 | 1.095 | 11.523 | −2.191 | C |
| 933 | CB | SER | 356 | 0.55 | 10.785 | −3.423 | C |
| 934 | OG | SER | 356 | 1.168 | 11.154 | −4.653 | O |
| 935 | C | SER | 356 | 1.148 | 13.048 | −2.386 | C |
| 936 | O | SER | 356 | 0.121 | 13.734 | −2.408 | O |
| 937 | H | SER | 356 | −0.62 | 10.805 | −1.092 | H |
| 938 | HA | SER | 356 | 2.117 | 11.173 | −2.047 | H |
| 939 | HB2 | SER | 356 | 0.676 | 9.712 | −3.272 | H |
| 940 | HB3 | SER | 356 | −0.518 | 10.991 | −3.505 | H |
| 941 | HG | SER | 356 | 2.148 | 11.161 | −4.615 | H |
| 942 | N | LEU | 357 | 2.368 | 13.58 | −2.513 | N |
| 943 | CA | LEU | 357 | 2.681 | 15.005 | −2.714 | C |
| 944 | CB | LEU | 357 | 3.865 | 15.363 | −1.797 | C |
| 945 | CG | LEU | 357 | 3.456 | 15.585 | −0.332 | C |
| 946 | CD1 | LEU | 357 | 4.702 | 15.569 | 0.547 | C |
| 947 | CD2 | LEU | 357 | 2.774 | 16.947 | −0.165 | C |
| 948 | C | LEU | 357 | 3.014 | 15.36 | −4.172 | C |
| 949 | O | LEU | 357 | 3.038 | 16.535 | −4.536 | O |
| 950 | H | LEU | 357 | 3.151 | 12.946 | −2.445 | H |
| 951 | HA | LEU | 357 | 1.82 | 15.614 | −2.443 | H |
| 952 | HB2 | LEU | 357 | 4.607 | 14.564 | −1.857 | H |
| 953 | HB3 | LEU | 357 | 4.344 | 16.274 | −2.16 | H |
| 954 | HG | LEU | 357 | 2.783 | 14.794 | −0.003 | H |
| 955 | HD11 | LEU | 357 | 5.219 | 14.615 | 0.439 | H |
| 956 | HD12 | LEU | 357 | 5.368 | 16.373 | 0.245 | H |
| 957 | HD13 | LEU | 357 | 4.425 | 15.689 | 1.591 | H |
| 958 | HD21 | LEU | 357 | 1.859 | 16.989 | −0.752 | H |
| 959 | HD22 | LEU | 357 | 2.514 | 17.102 | 0.88 | H |
| 960 | HD23 | LEU | 357 | 3.442 | 17.748 | −0.482 | H |
| 961 | N | ASP | 358 | 3.303 | 14.349 | −4.986 | N |
| 962 | CA | ASP | 358 | 3.683 | 14.447 | −6.389 | C |
| 963 | CB | ASP | 358 | 4.644 | 13.293 | −6.744 | C |
| 964 | CG | ASP | 358 | 4.224 | 11.87 | −6.317 | C |
| 965 | OD1 | ASP | 358 | 3.854 | 11.658 | −5.128 | O |
| 966 | OD2 | ASP | 358 | 4.43 | 10.953 | −7.145 | O1− |
| 967 | C | ASP | 358 | 2.457 | 14.546 | −7.315 | C |
| 968 | O | ASP | 358 | 1.51 | 13.764 | −7.211 | O |
| 969 | H | ASP | 358 | 3.25 | 13.404 | −4.62 | H |
| 970 | HA | ASP | 358 | 4.251 | 15.369 | −6.515 | H |
| 971 | HB2 | ASP | 358 | 4.817 | 13.312 | −7.821 | H |
| 972 | HB3 | ASP | 358 | 5.601 | 13.503 | −6.265 | H |
| 973 | N | ASN | 359 | 2.466 | 15.535 | −8.218 | N |
| 974 | CA | ASN | 359 | 1.357 | 15.825 | −9.133 | C |
| 975 | CB | ASN | 359 | 1.304 | 17.339 | −9.42 | C |
| 976 | CG | ASN | 359 | 0.404 | 18.088 | −8.452 | C |
| 977 | OD1 | ASN | 359 | −0.771 | 18.311 | −8.703 | O |
| 978 | ND2 | ASN | 359 | 0.892 | 18.555 | −7.329 | N |
| 979 | C | ASN | 359 | 1.435 | 14.961 | −10.41 | C |
| 980 | O | ASN | 359 | 2.465 | 14.966 | −11.099 | O |
| 981 | H | ASN | 359 | 3.292 | 16.11 | −8.293 | H |
| 982 | HA | ASN | 359 | 0.422 | 15.573 | −8.636 | H |
| 983 | HB2 | ASN | 359 | 2.302 | 17.775 | −9.41 | H |
| 984 | HB3 | ASN | 359 | 0.884 | 17.495 | −10.411 | H |
| 985 | HD21 | ASN | 359 | 1.865 | 18.431 | −7.069 | H |
| 986 | HD22 | ASN | 359 | 0.262 | 19.03 | −6.706 | H |
| 987 | N | ILE | 360 | 0.343 | 14.252 | −10.723 | N |
| 988 | CA | ILE | 360 | 0.171 | 13.307 | −11.839 | C |
| 989 | CB | ILE | 360 | −0.11 | 11.882 | −11.291 | C |
| 990 | CG2 | ILE | 360 | −0.243 | 10.847 | −12.425 | C |
| 991 | CG1 | ILE | 360 | 0.928 | 11.383 | −10.254 | C |
| 992 | CD1 | ILE | 360 | 2.384 | 11.316 | −10.734 | C |
| 993 | C | ILE | 360 | −0.973 | 13.781 | −12.753 | C |
| 994 | O | ILE | 360 | −2.08 | 14.055 | −12.285 | O |
| 995 | H | ILE | 360 | −0.431 | 14.298 | −10.065 | H |
| 996 | HA | ILE | 360 | 1.083 | 13.273 | −12.43 | H |
| 997 | HB | ILE | 360 | −1.072 | 11.913 | −10.775 | H |
| 998 | HG12 | ILE | 360 | 0.893 | 12.028 | −9.376 | H |
| 999 | HG13 | ILE | 360 | 0.634 | 10.388 | −9.918 | H |
| 1000 | HG21 | ILE | 360 | 0.665 | 10.816 | −13.025 | H |
| 1001 | HG22 | ILE | 360 | −0.432 | 9.858 | −12.006 | H |
| 1002 | HG23 | ILE | 360 | −1.084 | 11.099 | −13.072 | H |
| 1003 | HD11 | ILE | 360 | 3.01 | 10.968 | −9.911 | H |
| 1004 | HD12 | ILE | 360 | 2.482 | 10.626 | −11.572 | H |
| 1005 | HD13 | ILE | 360 | 2.732 | 12.303 | −11.029 | H |
| 1006 | N | THR | 361 | −0.732 | 13.813 | −14.067 | N |
| 1007 | CA | THR | 361 | −1.736 | 14.205 | −15.07 | C |
| 1008 | CB | THR | 361 | −1.123 | 15.099 | −16.159 | C |
| 1009 | CG2 | THR | 361 | −0.147 | 14.413 | −17.113 | C |
| 1010 | OG1 | THR | 361 | −2.132 | 15.696 | −16.94 | O |
| 1011 | C | THR | 361 | −2.475 | 12.989 | −15.638 | C |
| 1012 | O | THR | 361 | −1.898 | 11.909 | −15.774 | O |
| 1013 | H | THR | 361 | 0.136 | 13.412 | −14.402 | H |
| 1014 | HA | THR | 361 | −2.48 | 14.819 | −14.566 | H |
| 1015 | HB | THR | 361 | −0.584 | 15.905 | −15.665 | H |
| 1016 | HG21 | THR | 361 | 0.175 | 15.122 | −17.876 | H |
| 1017 | HG22 | THR | 361 | 0.73 | 14.082 | −16.561 | H |
| 1018 | HG23 | THR | 361 | −0.621 | 13.557 | −17.591 | H |
| 1019 | HG1 | THR | 361 | −2.288 | 16.567 | −16.525 | H |
| 1020 | N | HID | 362 | −3.756 | 13.163 | −15.971 | N |
| 1021 | CA | HID | 362 | −4.655 | 12.082 | −16.386 | C |
| 1022 | CB | HID | 362 | −6.101 | 12.601 | −16.468 | C |
| 1023 | CG | HID | 362 | −7.149 | 11.514 | −16.434 | C |
| 1024 | ND1 | HID | 362 | −7.928 | 11.192 | −15.352 | N |
| 1025 | CE1 | HID | 362 | −8.703 | 10.149 | −15.679 | C |
| 1026 | NE2 | HID | 362 | −8.512 | 9.811 | −16.968 | N |
| 1027 | CD2 | HID | 362 | −7.516 | 10.672 | −17.448 | C |
| 1028 | C | HID | 362 | −4.239 | 11.448 | −17.722 | C |
| 1029 | O | HID | 362 | −4.035 | 12.155 | −18.72 | O |
| 1030 | H | HID | 362 | −4.148 | 14.086 | −15.826 | H |
| 1031 | HA | HID | 362 | −4.615 | 11.317 | −15.613 | H |
| 1032 | HB2 | HID | 362 | −6.285 | 13.264 | −15.626 | H |
| 1033 | HB3 | HID | 362 | −6.225 | 13.179 | −17.383 | H |
| 1034 | HD1 | HID | 362 | −7.953 | 11.711 | −14.475 | H |
| 1035 | HD2 | HID | 362 | −7.102 | 10.674 | −18.444 | H |
| 1036 | HE1 | HID | 362 | −9.412 | 9.672 | −15.012 | H |
| 1037 | N | VAL | 363 | −4.258 | 10.114 | −17.788 | N |
| 1038 | CA | VAL | 363 | −4.047 | 9.333 | −19.019 | C |
| 1039 | CB | VAL | 363 | −2.59 | 8.821 | −19.102 | C |
| 1040 | CG1 | VAL | 363 | −2.325 | 8.173 | −20.469 | C |
| 1041 | CG2 | VAL | 363 | −1.535 | 9.924 | −18.926 | C |
| 1042 | C | VAL | 363 | −5.063 | 8.172 | −19.047 | C |
| 1043 | O | VAL | 363 | −4.937 | 7.244 | −18.247 | O |
| 1044 | H | VAL | 363 | −4.416 | 9.604 | −16.913 | H |
| 1045 | HA | VAL | 363 | −4.2 | 9.973 | −19.884 | H |
| 1046 | HB | VAL | 363 | −2.423 | 8.087 | −18.315 | H |
| 1047 | HG11 | VAL | 363 | −1.31 | 7.773 | −20.496 | H |
| 1048 | HG12 | VAL | 363 | −3.016 | 7.351 | −20.645 | H |
| 1049 | HG13 | VAL | 363 | −2.439 | 8.911 | −21.264 | H |
| 1050 | HG21 | VAL | 363 | −0.539 | 9.503 | −19.053 | H |
| 1051 | HG22 | VAL | 363 | −1.695 | 10.716 | −19.655 | H |
| 1052 | HG23 | VAL | 363 | −1.595 | 10.341 | −17.92 | H |
| 1053 | N | PRO | 364 | −6.102 | 8.176 | −19.914 | N |
| 1054 | CD | PRO | 364 | −6.471 | 9.247 | −20.831 | C |
| 1055 | CG | PRO | 364 | −7.988 | 9.152 | −20.982 | C |
| 1056 | CB | PRO | 364 | −8.234 | 7.651 | −20.878 | C |
| 1057 | CA | PRO | 364 | −7.198 | 7.19 | −19.842 | C |
| 1058 | C | PRO | 364 | −6.75 | 5.738 | −20.083 | C |
| 1059 | O | PRO | 364 | −7.006 | 4.848 | −19.265 | O |
| 1060 | HA | PRO | 364 | −7.651 | 7.252 | −18.853 | H |
| 1061 | HB2 | PRO | 364 | −8.033 | 7.201 | −21.851 | H |
| 1062 | HB3 | PRO | 364 | −9.253 | 7.429 | −20.559 | H |
| 1063 | HG2 | PRO | 364 | −8.326 | 9.556 | −21.936 | H |
| 1064 | HG3 | PRO | 364 | −8.48 | 9.66 | −20.151 | H |
| 1065 | HD2 | PRO | 364 | −5.99 | 9.082 | −21.797 | H |
| 1066 | HD3 | PRO | 364 | −6.207 | 10.231 | −20.446 | H |
| 1067 | N | GLY | 365 | −5.962 | 5.529 | −21.142 | N |
| 1068 | CA | GLY | 365 | −5.241 | 4.284 | −21.426 | C |
| 1069 | C | GLY | 365 | −3.864 | 4.235 | −20.752 | C |
| 1070 | O | GLY | 365 | −2.876 | 3.901 | −21.409 | O |
| 1071 | H | GLY | 365 | −5.792 | 6.314 | −21.755 | H |
| 1072 | HA2 | GLY | 365 | −5.827 | 3.434 | −21.076 | H |
| 1073 | HA3 | GLY | 365 | −5.115 | 4.18 | −22.501 | H |
| 1074 | N | GLY | 366 | −3.774 | 4.761 | −19.526 | N |
| 1075 | CA | GLY | 366 | −2.551 | 4.949 | −18.742 | C |
| 1076 | C | GLY | 366 | −2.68 | 4.471 | −17.291 | C |
| 1077 | O | GLY | 366 | −3.709 | 3.918 | −16.893 | O |
| 1078 | H | GLY | 366 | −4.63 | 5.077 | −19.091 | H |
| 1079 | HA2 | GLY | 366 | −1.719 | 4.421 | −19.208 | H |
| 1080 | HA3 | GLY | 366 | −2.291 | 6.002 | −18.724 | H |
| 1081 | N | GLY | 367 | −1.573 | 4.554 | −16.559 | N |
| 1082 | CA | GLY | 367 | −1.345 | 3.827 | −15.313 | C |
| 1083 | C | GLY | 367 | −2.028 | 4.421 | −14.081 | C |
| 1084 | O | GLY | 367 | −2.552 | 5.533 | −14.094 | O |
| 1085 | H | GLY | 367 | −0.793 | 5.059 | −16.962 | H |
| 1086 | HA2 | GLY | 367 | −1.706 | 2.807 | −15.437 | H |
| 1087 | HA3 | GLY | 367 | −0.273 | 3.777 | −15.117 | H |
| 1088 | N | ASN | 368 | −1.948 | 3.675 | −12.983 | N |
| 1089 | CA | ASN | 368 | −2.581 | 4.026 | −11.715 | C |
| 1090 | CB | ASN | 368 | −2.83 | 2.71 | −10.954 | C |
| 1091 | CG | ASN | 368 | −3.89 | 1.826 | −11.598 | C |
| 1092 | OD1 | ASN | 368 | −4.537 | 2.16 | −12.581 | O |
| 1093 | ND2 | ASN | 368 | −4.129 | 0.654 | −11.066 | N |
| 1094 | C | ASN | 368 | −1.748 | 5.055 | −10.915 | C |
| 1095 | O | ASN | 368 | −0.525 | 4.909 | −10.81 | O |
| 1096 | H | ASN | 368 | −1.518 | 2.76 | −13.065 | H |
| 1097 | HA | ASN | 368 | −3.546 | 4.487 | −11.932 | H |
| 1098 | HB2 | ASN | 368 | −1.901 | 2.145 | −10.882 | H |
| 1099 | HB3 | ASN | 368 | −3.154 | 2.935 | −9.944 | H |
| 1100 | HD21 | ASN | 368 | −3.56 | 0.253 | −10.33 | H |
| 1101 | HD22 | ASN | 368 | −4.885 | 0.124 | −11.48 | H |
| 1102 | N | LYS | 369 | −2.401 | 6.049 | −10.292 | N |
| 1103 | CA | LYS | 369 | −1.797 | 7.102 | −9.44 | C |
| 1104 | CB | LYS | 369 | −2.864 | 8.168 | −9.11 | C |
| 1105 | CG | LYS | 369 | −4.001 | 7.735 | −8.148 | C |
| 1106 | CD | LYS | 369 | −3.729 | 7.865 | −6.637 | C |
| 1107 | CE | LYS | 369 | −3.436 | 9.312 | −6.232 | C |
| 1108 | NZ | LYS | 369 | −2.751 | 9.419 | −4.929 | N1+ |
| 1109 | C | LYS | 369 | −1.119 | 6.538 | −8.183 | C |
| 1110 | O | LYS | 369 | −1.519 | 5.475 | −7.707 | O |
| 1111 | H | LYS | 369 | −3.408 | 6.088 | −10.427 | H |
| 1112 | HA | LYS | 369 | −1.017 | 7.588 | −10.027 | H |
| 1113 | HB2 | LYS | 369 | −2.365 | 9.056 | −8.729 | H |
| 1114 | HB3 | LYS | 369 | −3.328 | 8.466 | −10.05 | H |
| 1115 | HG2 | LYS | 369 | −4.882 | 8.334 | −8.379 | H |
| 1116 | HG3 | LYS | 369 | −4.259 | 6.697 | −8.348 | H |
| 1117 | HD2 | LYS | 369 | −4.607 | 7.521 | −6.088 | H |
| 1118 | HD3 | LYS | 369 | −2.897 | 7.226 | −6.362 | H |
| 1119 | HE2 | LYS | 369 | −2.799 | 9.769 | −6.989 | H |
| 1120 | HE3 | LYS | 369 | −4.373 | 9.873 | −6.206 | H |
| 1121 | HZ | LYS | 369 | −2.542 | 10.391 | −4.717 | H |
| 1122 | HZ2 | LYS | 369 | −3.344 | 9.102 | −4.164 | H |
| 1123 | HZ3 | LYS | 369 | −1.889 | 8.882 | −4.921 | H |
| 1124 | N | LYS | 370 | −0.136 | 7.236 | −7.594 | N |
| 1125 | CA | LYS | 370 | 0.67 | 6.677 | −6.488 | C |
| 1126 | CB | LYS | 370 | 1.914 | 7.544 | −6.193 | C |
| 1127 | CG | LYS | 370 | 2.78 | 6.887 | −5.1 | C |
| 1128 | CD | LYS | 370 | 4.099 | 7.614 | −4.813 | C |
| 1129 | CE | LYS | 370 | 4.794 | 7 | −3.584 | C |
| 1130 | NZ | LYS | 370 | 5.236 | 5.6 | −3.818 | N1+ |
| 1131 | C | LYS | 370 | −0.169 | 6.426 | −5.228 | C |
| 1132 | O | LYS | 370 | −0.9 | 7.306 | −4.767 | O |
| 1133 | H | LYS | 370 | 0.049 | 8.184 | −7.904 | H |
| 1134 | HA | LYS | 370 | 1.032 | 5.704 | −6.827 | H |
| 1135 | HB2 | LYS | 370 | 2.505 | 7.648 | −7.104 | H |
| 1136 | HB3 | LYS | 370 | 1.604 | 8.536 | −5.86 | H |
| 1137 | HG2 | LYS | 370 | 2.214 | 6.868 | −4.17 | H |
| 1138 | HG3 | LYS | 370 | 2.999 | 5.86 | −5.396 | H |
| 1139 | HD2 | LYS | 370 | 4.754 | 7.562 | −5.685 | H |
| 1140 | HD3 | LYS | 370 | 3.889 | 8.665 | −4.605 | H |
| 1141 | HE2 | LYS | 370 | 5.663 | 7.618 | −3.339 | H |
| 1142 | HE3 | LYS | 370 | 4.102 | 7.038 | −2.736 | H |
| 1143 | HZ1 | LYS | 370 | 5.673 | 5.196 | −2.99 | H |
| 1144 | HZ2 | LYS | 370 | 4.462 | 4.98 | −4.047 | H |
| 1145 | HZ3 | LYS | 370 | 5.92 | 5.574 | −4.571 | H |
| 1146 | N | ILE | 371 | −0.009 | 5.232 | −4.649 | N |
| 1147 | CA | ILE | 371 | −0.668 | 4.808 | −3.405 | C |
| 1148 | CB | ILE | 371 | −0.386 | 3.313 | −3.105 | C |
| 1149 | CG2 | ILE | 371 | −1.069 | 2.871 | −1.796 | C |
| 1150 | CG1 | ILE | 371 | −0.84 | 2.413 | −4.282 | C |
| 1151 | CD1 | ILE | 371 | −0.501 | 0.926 | −4.115 | C |
| 1152 | C | ILE | 371 | −0.244 | 5.722 | −2.244 | C |
| 1153 | O | ILE | 371 | 0.936 | 6.025 | −2.058 | O |
| 1154 | H | ILE | 371 | 0.615 | 4.577 | −5.091 | H |
| 1155 | HA | ILE | 371 | −1.744 | 4.92 | −3.545 | H |
| 1156 | HB | ILE | 371 | 0.689 | 3.192 | −2.978 | H |
| 1157 | HG12 | ILE | 371 | −1.917 | 2.515 | −4.424 | H |
| 1158 | HG13 | ILE | 371 | −0.349 | 2.742 | −5.197 | H |
| 1159 | HG21 | ILE | 371 | −0.812 | 1.838 | −1.562 | H |
| 1160 | HG22 | ILE | 371 | −0.718 | 3.465 | −0.954 | H |
| 1161 | HG23 | ILE | 371 | −2.153 | 2.957 | −1.886 | H |
| 1162 | HD11 | ILE | 371 | 0.55 | 0.815 | −3.859 | H |
| 1163 | HD12 | ILE | 371 | −1.114 | 0.475 | −3.336 | H |
| 1164 | HD13 | ILE | 371 | −0.691 | 0.403 | −5.053 | H |
| 1165 | N | GLU | 372 | −1.229 | 6.158 | −1.458 | N |
| 1166 | CA | GLU | 372 | −1.084 | 7.207 | −0.439 | C |
| 1167 | CB | GLU | 372 | −2.468 | 7.488 | 0.17 | C |
| 1168 | CG | GLU | 372 | −3.568 | 7.904 | −0.826 | C |
| 1169 | CD | GLU | 372 | −3.292 | 9.211 | −1.583 | C |
| 1170 | OE1 | GLU | 372 | −4.048 | 9.506 | −2.539 | O |
| 1171 | OE2 | GLU | 372 | −2.324 | 9.933 | −1.254 | O1− |
| 1172 | C | GLU | 372 | −0.118 | 6.846 | 0.701 | C |
| 1173 | O | GLU | 372 | 0.622 | 7.706 | 1.177 | O |
| 1174 | H | GLU | 372 | −2.168 | 5.871 | −1.688 | H |
| 1175 | HA | GLU | 372 | −0.705 | 8.116 | −0.907 | H |
| 1176 | HB2 | GLU | 372 | −2.81 | 6.589 | 0.685 | H |
| 1177 | HB3 | GLU | 372 | −2.352 | 8.265 | 0.919 | H |
| 1178 | HG2 | GLU | 372 | −3.72 | 7.097 | −1.546 | H |
| 1179 | HG3 | GLU | 372 | −4.499 | 8.017 | −0.268 | H |
| 1180 | N | THR | 373 | −0.103 | 5.569 | 1.094 | N |
| 1181 | CA | THR | 373 | 0.553 | 5.012 | 2.286 | C |
| 1182 | CB | THR | 373 | 0.449 | 3.477 | 2.216 | C |
| 1183 | CG2 | THR | 373 | 0.91 | 2.759 | 3.483 | C |
| 1184 | OG1 | THR | 373 | −0.889 | 3.093 | 1.969 | O |
| 1185 | C | THR | 373 | 2.024 | 5.425 | 2.421 | C |
| 1186 | O | THR | 373 | 2.856 | 5.061 | 1.59 | O |
| 1187 | H | THR | 373 | −0.708 | 4.933 | 0.597 | H |
| 1188 | HA | THR | 373 | 0.012 | 5.353 | 3.167 | H |
| 1189 | HB | THR | 373 | 1.055 | 3.123 | 1.381 | H |
| 1190 | HG21 | THR | 373 | 0.352 | 3.114 | 4.345 | H |
| 1191 | HG22 | THR | 373 | 0.746 | 1.689 | 3.366 | H |
| 1192 | HG23 | THR | 373 | 1.975 | 2.929 | 3.64 | H |
| 1193 | HG1 | THR | 373 | −1.437 | 3.366 | 2.731 | H |
| 1194 | N | HIE | 374 | 2.371 | 6.118 | 3.511 | N |
| 1195 | CA | HIE | 374 | 3.731 | 6.573 | 3.845 | C |
| 1196 | CB | HIE | 374 | 3.852 | 8.084 | 3.572 | C |
| 1197 | CG | HIE | 374 | 3.066 | 8.981 | 4.497 | C |
| 1198 | ND1 | HIE | 374 | 1.949 | 9.734 | 4.132 | N |
| 1199 | CE1 | HIE | 374 | 1.625 | 10.465 | 5.209 | C |
| 1200 | NE2 | HIE | 374 | 2.473 | 10.21 | 6.219 | N |
| 1201 | CD2 | HIE | 374 | 3.391 | 9.279 | 5.788 | C |
| 1202 | C | HIE | 374 | 4.205 | 6.174 | 5.257 | C |
| 1203 | O | HIE | 374 | 5.304 | 6.564 | 5.662 | O |
| 1204 | H | HIE | 374 | 1.627 | 6.449 | 4.119 | H |
| 1205 | HA | HIE | 374 | 4.433 | 6.08 | 3.172 | H |
| 1206 | HB2 | HIE | 374 | 4.902 | 8.37 | 3.637 | H |
| 1207 | HB3 | HIE | 374 | 3.529 | 8.283 | 2.55 | H |
| 1208 | HD2 | HIE | 374 | 4.239 | 8.894 | 6.337 | H |
| 1209 | HE2 | HIE | 374 | 2.458 | 10.661 | 7.123 | H |
| 1210 | HE1 | HIE | 374 | 0.803 | 11.167 | 5.252 | H |
| 1211 | N | LYS | 375 | 3.428 | 5.35 | 5.978 | N |
| 1212 | CA | LYS | 375 | 3.809 | 4.689 | 7.241 | C |
| 1213 | CB | LYS | 375 | 2.845 | 5.081 | 8.379 | C |
| 1214 | CG | LYS | 375 | 2.848 | 6.582 | 8.714 | C |
| 1215 | CD | LYS | 375 | 2.415 | 6.805 | 10.173 | C |
| 1216 | CE | LYS | 375 | 2.116 | 8.282 | 10.429 | C |
| 1217 | NZ | LYS | 375 | 2.008 | 8.598 | 11.874 | N1+ |
| 1218 | C | LYS | 375 | 3.91 | 3.16 | 7.117 | C |
| 1219 | O | LYS | 375 | 3.457 | 2.563 | 6.138 | O |
| 1220 | H | LYS | 375 | 2.515 | 5.137 | 5.601 | H |
| 1221 | HA | LYS | 375 | 4.808 | 5.027 | 7.515 | H |
| 1222 | HB2 | LYS | 375 | 1.828 | 4.777 | 8.121 | H |
| 1223 | HB3 | LYS | 375 | 3.135 | 4.532 | 9.276 | H |
| 1224 | HG2 | LYS | 375 | 3.852 | 6.99 | 8.585 | H |
| 1225 | HG3 | LYS | 375 | 2.168 | 7.1 | 8.036 | H |
| 1226 | HD2 | LYS | 375 | 1.519 | 6.22 | 10.384 | H |
| 1227 | HD3 | LYS | 375 | 3.218 | 6.474 | 10.833 | H |
| 1228 | HE2 | LYS | 375 | 2.915 | 8.89 | 9.994 | H |
| 1229 | HE3 | LYS | 375 | 1.183 | 8.541 | 9.922 | H |
| 1230 | HZ1 | LYS | 375 | 1.29 | 8.048 | 12.343 | H |
| 1231 | HZ2 | LYS | 375 | 2.908 | 8.477 | 12.335 | H |
| 1232 | HZ3 | LYS | 375 | 1.803 | 9.591 | 11.966 | H |
| 1233 | N | LEU | 376 | 4.489 | 2.536 | 8.144 | N |
| 1234 | CA | LEU | 376 | 4.687 | 1.087 | 8.281 | C |
| 1235 | CB | LEU | 376 | 6.082 | 0.846 | 8.892 | C |
| 1236 | CG | LEU | 376 | 7.246 | 1.519 | 8.133 | C |
| 1237 | CD1 | LEU | 376 | 8.548 | 1.284 | 8.892 | C |
| 1238 | CD2 | LEU | 376 | 7.379 | 0.987 | 6.705 | C |
| 1239 | C | LEU | 376 | 3.577 | 0.437 | 9.133 | C |
| 1240 | O | LEU | 376 | 3.058 | 1.078 | 10.047 | O |
| 1241 | H | LEU | 376 | 4.852 | 3.123 | 8.887 | H |
| 1242 | HA | LEU | 376 | 4.657 | 0.626 | 7.293 | H |
| 1243 | HB2 | LEU | 376 | 6.076 | 1.227 | 9.915 | H |
| 1244 | HB3 | LEU | 376 | 6.263 | −0.229 | 8.937 | H |
| 1245 | HG | LEU | 376 | 7.089 | 2.596 | 8.084 | H |
| 1246 | HD11 | LEU | 376 | 8.496 | 1.771 | 9.866 | H |
| 1247 | HD12 | LEU | 376 | 8.711 | 0.222 | 9.032 | H |
| 1248 | HD13 | LEU | 376 | 9.383 | 1.713 | 8.338 | H |
| 1249 | HD21 | LEU | 376 | 7.445 | −0.099 | 6.706 | H |
| 1250 | HD22 | LEU | 376 | 6.523 | 1.3 | 6.109 | H |
| 1251 | HD23 | LEU | 376 | 8.277 | 1.403 | 6.248 | H |
| 1252 | N | THR | 377 | 3.229 | −0.833 | 8.87 | N |
| 1253 | CA | THR | 377 | 2.053 | −1.515 | 9.479 | C |
| 1254 | CB | THR | 377 | 0.91 | −1.594 | 8.449 | C |
| 1255 | CG2 | THR | 377 | 1.154 | −2.619 | 7.341 | C |
| 1256 | OG1 | THR | 377 | −0.31 | −1.923 | 9.066 | O |
| 1257 | C | THR | 377 | 2.318 | −2.896 | 10.115 | C |
| 1258 | O | THR | 377 | 1.376 | −3.562 | 10.552 | O |
| 1259 | H | THR | 377 | 3.681 | −1.281 | 8.085 | H |
| 1260 | HA | THR | 377 | 1.683 | −0.894 | 10.296 | H |
| 1261 | HB | THR | 377 | 0.797 | −0.612 | 7.99 | H |
| 1262 | HG21 | THR | 377 | 1.137 | −3.633 | 7.743 | H |
| 1263 | HG22 | THR | 377 | 0.372 | −2.529 | 6.587 | H |
| 1264 | HG23 | THR | 377 | 2.119 | −2.437 | 6.871 | H |
| 1265 | HG1 | THR | 377 | −0.127 | −2.677 | 9.648 | H |
| 1266 | N | PHE | 378 | 3.572 | −3.356 | 10.153 | N |
| 1267 | CA | PHE | 378 | 3.952 | −4.685 | 10.657 | C |
| 1268 | CB | PHE | 378 | 4.648 | −5.479 | 9.541 | C |
| 1269 | CG | PHE | 378 | 3.813 | −5.665 | 8.288 | C |
| 1270 | CD1 | PHE | 378 | 2.693 | −6.517 | 8.313 | C |
| 1271 | CE1 | PHE | 378 | 1.923 | −6.708 | 7.153 | C |
| 1272 | CZ | PHE | 378 | 2.273 | −6.054 | 5.96 | C |
| 1273 | CE2 | PHE | 378 | 3.389 | −5.199 | 5.932 | C |
| 1274 | CD2 | PHE | 378 | 4.153 | −4.997 | 7.095 | C |
| 1275 | C | PHE | 378 | 4.852 | −4.58 | 11.894 | C |
| 1276 | O | PHE | 378 | 5.662 | −3.654 | 11.983 | O |
| 1277 | H | PHE | 378 | 4.315 | −2.755 | 9.833 | H |
| 1278 | HA | PHE | 378 | 3.059 | −5.238 | 10.947 | H |
| 1279 | HB2 | PHE | 378 | 5.58 | −4.976 | 9.28 | H |
| 1280 | HB3 | PHE | 378 | 4.909 | −6.467 | 9.924 | H |
| 1281 | HD1 | PHE | 378 | 2.42 | −7.028 | 9.225 | H |
| 1282 | HD2 | PHE | 378 | 5.021 | −4.353 | 7.063 | H |
| 1283 | HE1 | PHE | 378 | 1.059 | −7.359 | 7.184 | H |
| 1284 | HE2 | PHE | 378 | 3.67 | −4.715 | 5.009 | H |
| 1285 | HZ | PHE | 378 | 1.683 | −6.212 | 5.067 | H |
| 1286 | N | ARG | 379 | 4.757 | −5.538 | 12.826 | N |
| 1287 | CA | ARG | 379 | 5.738 | −5.681 | 13.92 | C |
| 1288 | CB | ARG | 379 | 5.147 | −6.446 | 15.123 | C |
| 1289 | CG | ARG | 379 | 5.333 | −7.972 | 15.132 | C |
| 1290 | CD | ARG | 379 | 4.674 | −8.726 | 13.972 | C |
| 1291 | NE | ARG | 379 | 4.809 | −10.17 | 14.201 | N |
| 1292 | CZ | ARG | 379 | 3.997 | −11.122 | 13.79 | C |
| 1293 | NH1 | ARG | 379 | 3.16 | −10.997 | 12.8 | N |
| 1294 | NH2 | ARG | 379 | 4.005 | −12.259 | 14.409 | N1+ |
| 1295 | C | ARG | 379 | 7.065 | −6.248 | 13.403 | C |
| 1296 | O | ARG | 379 | 7.099 | −6.892 | 12.357 | O |
| 1297 | H | ARG | 379 | 4.046 | −6.244 | 12.717 | H |
| 1298 | HA | ARG | 379 | 5.968 | −4.677 | 14.283 | H |
| 1299 | HB2 | ARG | 379 | 5.635 | −6.067 | 16.023 | H |
| 1300 | HB3 | ARG | 379 | 4.085 | −6.213 | 15.22 | H |
| 1301 | HG2 | ARG | 379 | 6.398 | −8.205 | 15.144 | H |
| 1302 | HG3 | ARG | 379 | 4.912 | −8.346 | 16.065 | H |
| 1303 | HD2 | ARG | 379 | 3.617 | −8.456 | 13.932 | H |
| 1304 | HD3 | ARG | 379 | 5.153 | −8.456 | 13.03 | H |
| 1305 | HE | ARG | 379 | 5.536 | −10.466 | 14.839 | H |
| 1306 | HH11 | ARG | 379 | 2.607 | −11.804 | 12.542 | H |
| 1307 | HH12 | ARG | 379 | 3.237 | −10.214 | 12.165 | H |
| 1308 | HH21 | ARG | 379 | 3.368 | −12.977 | 14.092 | H |
| 1309 | HH22 | ARG | 379 | 4.409 | −12.292 | 15.336 | H |
| 1310 | N | GLU | 380 | 8.158 | −6.012 | 14.117 | N |
| 1311 | CA | GLU | 380 | 9.531 | −6.247 | 13.64 | C |
| 1312 | CB | GLU | 380 | 10.473 | −5.223 | 14.308 | C |
| 1313 | CG | GLU | 380 | 10.918 | −5.532 | 15.752 | C |
| 1314 | CD | GLU | 380 | 9.776 | −5.749 | 16.759 | C |
| 1315 | OE1 | GLU | 380 | 8.713 | −5.095 | 16.645 | O |
| 1316 | OE2 | GLU | 380 | 9.949 | −6.589 | 17.669 | O1− |
| 1317 | C | GLU | 380 | 10.05 | −7.688 | 13.804 | C |
| 1318 | O | GLU | 380 | 11.068 | −8.049 | 13.211 | O |
| 1319 | H | GLU | 380 | 8.058 | −5.508 | 14.999 | H |
| 1320 | HA | GLU | 380 | 9.553 | −6.041 | 12.569 | H |
| 1321 | HB2 | GLU | 380 | 11.376 | −5.156 | 13.698 | H |
| 1322 | HB3 | GLU | 380 | 10.002 | −4.239 | 14.283 | H |
| 1323 | HG2 | GLU | 380 | 11.554 | −6.42 | 15.734 | H |
| 1324 | HG3 | GLU | 380 | 11.536 | −4.703 | 16.101 | H |
| 1325 | N | ASN | 381 | 9.378 | −8.507 | 14.619 | N |
| 1326 | CA | ASN | 381 | 9.888 | −9.793 | 15.1 | C |
| 1327 | CB | ASN | 381 | 10.277 | −9.59 | 16.576 | C |
| 1328 | CG | ASN | 381 | 10.946 | −10.77 | 17.253 | C |
| 1329 | OD1 | ASN | 381 | 11.18 | −11.824 | 16.676 | O |
| 1330 | ND2 | ASN | 381 | 11.308 | −10.609 | 18.503 | N |
| 1331 | C | ASN | 381 | 8.886 | −10.941 | 14.87 | C |
| 1332 | C | ASN | 381 | 7.697 | −10.829 | 15.186 | O |
| 1333 | H | ASN | 381 | 8.556 | −8.138 | 15.073 | H |
| 1334 | HA | ASN | 381 | 10.798 | −10.044 | 14.553 | H |
| 1335 | HB2 | ASN | 381 | 10.977 | −8.756 | 16.634 | H |
| 1336 | HB3 | ASN | 381 | 9.395 | −9.315 | 17.151 | H |
| 1337 | HD21 | ASN | 381 | 11.177 | −9.696 | 18.934 | H |
| 1338 | HD22 | ASN | 381 | 11.842 | −11.336 | 18.94 | H |
| 1339 | N | ALA | 382 | 9.396 | −12.078 | 14.381 | N |
| 1340 | CA | ALA | 382 | 8.625 | −13.265 | 14.012 | C |
| 1341 | CB | ALA | 382 | 9.612 | −14.306 | 13.473 | C |
| 1342 | C | ALA | 382 | 7.778 | −13.868 | 15.151 | C |
| 1343 | O | ALA | 382 | 6.683 | −14.383 | 14.897 | O |
| 1344 | H | ALA | 382 | 10.388 | −12.099 | 14.187 | H |
| 1345 | HA | ALA | 382 | 7.945 | −12.991 | 13.209 | H |
| 1346 | HB1 | ALA | 382 | 10.141 | −13.902 | 12.61 | H |
| 1347 | HB2 | ALA | 382 | 10.336 | −14.576 | 14.244 | H |
| 1348 | HB3 | ALA | 382 | 9.069 | −15.202 | 13.169 | H |
| 1349 | N | LYS | 383 | 8.247 | −13.783 | 16.404 | N |
| 1350 | CA | LYS | 383 | 7.517 | −14.292 | 17.581 | C |
| 1351 | CB | LYS | 383 | 8.48 | −15.014 | 18.536 | C |
| 1352 | CG | LYS | 383 | 9.477 | −14.065 | 19.214 | C |
| 1353 | CD | LYS | 383 | 10.385 | −14.833 | 20.177 | C |
| 1354 | CE | LYS | 383 | 11.402 | −13.86 | 20.769 | C |
| 1355 | NZ | LYS | 383 | 12.327 | −14.546 | 21.691 | N1+ |
| 1356 | C | LYS | 383 | 6.653 | −13.25 | 18.302 | C |
| 1357 | O | LYS | 383 | 5.815 | −13.633 | 19.116 | O |
| 1358 | H | LYS | 383 | 9.171 | −13.385 | 16.536 | H |
| 1359 | HA | LYS | 383 | 6.81 | −15.048 | 17.242 | H |
| 1360 | HB2 | LYS | 383 | 7.895 | −15.52 | 19.306 | H |
| 1361 | HB3 | LYS | 383 | 9.029 | −15.776 | 17.979 | H |
| 1362 | HG2 | LYS | 383 | 10.089 | −13.58 | 18.453 | H |
| 1363 | HG3 | LYS | 383 | 8.939 | −13.299 | 19.774 | H |
| 1364 | HD2 | LYS | 383 | 9.782 | −15.271 | 20.974 | H |
| 1365 | HD3 | LYS | 383 | 10.904 | −15.629 | 19.64 | H |
| 1366 | HE2 | LYS | 383 | 11.971 | −13.406 | 19.952 | H |
| 1367 | HE3 | LYS | 383 | 10.864 | −13.072 | 21.304 | H |
| 1368 | HZ1 | LYS | 383 | 11.819 | −15.028 | 22.429 | H |
| 1369 | HZ2 | LYS | 383 | 12.919 | −15.203 | 21.189 | H |
| 1370 | HZ3 | LYS | 383 | 12.967 | −13.878 | 22.111 | H |
| 1371 | N | ALA | 384 | 6.825 | −11.959 | 18.006 | N |
| 1372 | CA | ALA | 384 | 6.026 | −10.883 | 18.599 | C |
| 1373 | CB | ALA | 384 | 6.716 | −9.542 | 18.323 | C |
| 1374 | C | ALA | 384 | 4.566 | −10.924 | 18.104 | C |
| 1375 | O | ALA | 384 | 4.267 | −11.56 | 17.087 | O |
| 1376 | H | ALA | 384 | 7.463 | −11.716 | 17.263 | H |
| 1377 | HA | ALA | 384 | 6.004 | −11.023 | 19.682 | H |
| 1378 | HB1 | ALA | 384 | 7.689 | −9.521 | 18.815 | H |
| 1379 | HB2 | ALA | 384 | 6.847 | −9.396 | 17.253 | H |
| 1380 | HB3 | ALA | 384 | 6.114 | −8.722 | 18.717 | H |
| 1381 | N | LYS | 385 | 3.65 | −10.274 | 18.833 | N |
| 1382 | CA | LYS | 385 | 2.2 | −10.289 | 18.553 | C |
| 1383 | CB | LYS | 385 | 1.422 | −10.295 | 19.885 | C |
| 1384 | CG | LYS | 385 | 1.658 | −11.611 | 20.647 | C |
| 1385 | CD | LYS | 385 | 0.686 | −11.789 | 21.82 | C |
| 1386 | CE | LYS | 385 | 1.005 | −13.101 | 22.549 | C |
| 1387 | NZ | LYS | 385 | −0.032 | −13.429 | 23.551 | N1+ |
| 1388 | C | LYS | 385 | 1.777 | −9.151 | 17.612 | C |
| 1389 | O | LYS | 385 | 2.583 | −8.298 | 17.249 | O |
| 1390 | H | LYS | 385 | 3.981 | −9.678 | 19.586 | H |
| 1391 | HA | LYS | 385 | 1.953 | −11.207 | 18.02 | H |
| 1392 | HB2 | LYS | 385 | 1.732 | −9.449 | 20.502 | H |
| 1393 | HB3 | LYS | 385 | 0.356 | −10.194 | 19.683 | H |
| 1394 | HG2 | LYS | 385 | 1.523 | −12.448 | 19.96 | H |
| 1395 | HG3 | LYS | 385 | 2.682 | −11.631 | 21.024 | H |
| 1396 | HD2 | LYS | 385 | 0.786 | −10.95 | 22.513 | H |
| 1397 | HD3 | LYS | 385 | −0.336 | −11.818 | 21.437 | H |
| 1398 | HE2 | LYS | 385 | 1.069 | −13.909 | 21.814 | H |
| 1399 | HE3 | LYS | 385 | 1.979 | −13.003 | 23.037 | H |
| 1400 | HZ1 | LYS | 385 | 0.169 | −14.3 | 24.032 | H |
| 1401 | HZ2 | LYS | 385 | −0.113 | −12.692 | 24.251 | H |
| 1402 | HZ3 | LYS | 385 | −0.935 | −13.568 | 23.102 | H |
| 1403 | N | THR | 386 | 0.517 | −9.161 | 17.17 | N |
| 1404 | CA | THR | 386 | −0.09 | −8.058 | 16.412 | C |
| 1405 | CB | THR | 386 | 0.078 | −8.223 | 14.888 | C |
| 1406 | CG2 | THR | 386 | −0.675 | −9.412 | 14.301 | C |
| 1407 | OG1 | THR | 386 | −0.344 | −7.064 | 14.186 | O |
| 1408 | C | THR | 386 | −1.552 | −7.835 | 16.787 | C |
| 1409 | O | THR | 386 | −2.302 | −8.766 | 17.082 | O |
| 1410 | H | THR | 386 | −0.099 | −9.909 | 17.474 | H |
| 1411 | HA | THR | 386 | 0.445 | −7.151 | 16.692 | H |
| 1412 | HB | THR | 386 | 1.14 | −8.363 | 14.683 | H |
| 1413 | HG21 | THR | 386 | −1.752 | −9.287 | 14.423 | H |
| 1414 | HG22 | THR | 386 | −0.443 | −9.497 | 13.239 | H |
| 1415 | HG23 | THR | 386 | −0.359 | −10.323 | 14.806 | H |
| 1416 | HG1 | THR | 386 | −1.314 | −7.006 | 14.253 | H |
| 1417 | N | ASP | 387 | −1.941 | −6.568 | 16.724 | N |
| 1418 | CA | ASP | 387 | −3.299 | −6.053 | 16.79 | C |
| 1419 | CB | ASP | 387 | −3.238 | −4.574 | 17.233 | C |
| 1420 | CG | ASP | 387 | −2.35 | −3.649 | 16.372 | C |
| 1421 | OD1 | ASP | 387 | −1.201 | −4.019 | 16.023 | O |
| 1422 | OD2 | ASP | 387 | −2.791 | −2.516 | 16.069 | O1− |
| 1423 | C | ASP | 387 | −4.053 | −6.248 | 15.459 | C |
| 1424 | O | ASP | 387 | −3.456 | −6.206 | 14.375 | O |
| 1425 | H | ASP | 387 | −1.25 | −5.869 | 16.471 | H |
| 1426 | HA | ASP | 387 | −3.833 | −6.606 | 17.56 | H |
| 1427 | HB2 | ASP | 387 | −4.255 | −4.181 | 17.252 | H |
| 1428 | HB3 | ASP | 387 | −2.861 | −4.541 | 18.257 | H |
| 1429 | N | HIE | 388 | −5.372 | −6.462 | 15.536 | N |
| 1430 | CA | HIE | 388 | −6.281 | −6.542 | 14.383 | C |
| 1431 | CB | HIE | 388 | −6.257 | −7.967 | 13.771 | C |
| 1432 | CG | HIE | 388 | −7.094 | −9.041 | 14.435 | C |
| 1433 | ND1 | HIE | 388 | −6.62 | −10.102 | 15.22 | N |
| 1434 | CE1 | HIE | 388 | −7.692 | −10.868 | 15.489 | C |
| 1435 | NE2 | HIE | 388 | −8.792 | −10.359 | 14.913 | N |
| 1436 | CD2 | HIE | 388 | −8.43 | −9.225 | 14.227 | C |
| 1437 | C | HIE | 388 | −7.704 | −6.043 | 14.714 | C |
| 1438 | O | HIE | 388 | −8.036 | −5.796 | 15.874 | O |
| 1439 | H | HIE | 388 | −5.795 | −6.52 | 16.46 | H |
| 1440 | HA | HIE | 388 | −5.898 | −5.861 | 13.622 | H |
| 1441 | HB2 | HIE | 388 | −6.605 | −7.891 | 12.741 | H |
| 1442 | HB3 | HIE | 388 | −5.225 | −8.317 | 13.724 | H |
| 1443 | HD2 | HIE | 388 | −9.071 | −8.617 | 13.606 | H |
| 1444 | HE2 | HIE | 388 | −9.73 | −10.731 | 15.001 | H |
| 1445 | HE1 | HIE | 388 | −7.681 | −11.76 | 16.103 | H |
| 1446 | N | GLY | 389 | −8.534 | −5.88 | 13.678 | N |
| 1447 | CA | GLY | 389 | −9.949 | −5.486 | 13.737 | C |
| 1448 | C | GLY | 389 | −10.872 | −6.603 | 14.242 | C |
| 1449 | O | GLY | 389 | −10.628 | −7.171 | 15.307 | O |
| 1450 | H | GLY | 389 | −8.146 | −6.019 | 12.752 | H |
| 1451 | HA2 | GLY | 389 | −10.058 | −4.624 | 14.392 | H |
| 1452 | HA3 | GLY | 389 | −10.271 | −5.185 | 12.739 | H |
| 1453 | N | ALA | 390 | −11.933 | −6.934 | 13.5 | N |
| 1454 | CA | ALA | 390 | −12.845 | −8.038 | 13.829 | C |
| 1455 | CB | ALA | 390 | −14.197 | −7.776 | 13.152 | C |
| 1456 | C | ALA | 390 | −12.289 | −9.444 | 13.487 | C |
| 1457 | O | ALA | 390 | −11.409 | −9.613 | 12.632 | O |
| 1458 | H | ALA | 390 | −12.141 | −6.386 | 12.672 | H |
| 1459 | HA | ALA | 390 | −13.018 | −8.019 | 14.907 | H |
| 1460 | HB1 | ALA | 390 | −14.083 | −7.8 | 12.069 | H |
| 1461 | HB2 | ALA | 390 | −14.916 | −8.54 | 13.45 | H |
| 1462 | HB3 | ALA | 390 | −14.576 | −6.797 | 13.451 | H |
| 1463 | N | GLU | 391 | −12.83 | −10.471 | 14.157 | N |
| 1464 | CA | GLU | 391 | −12.433 | −11.891 | 14.063 | C |
| 1465 | CB | GLU | 391 | −11.893 | −12.318 | 15.437 | C |
| 1466 | CG | GLU | 391 | −11.31 | −13.733 | 15.478 | C |
| 1467 | CD | GLU | 391 | −9.913 | −13.835 | 14.866 | C |
| 1468 | OE1 | GLU | 391 | −8.972 | −14.255 | 15.579 | O |
| 1469 | OE2 | GLU | 391 | −9.749 | −13.547 | 13.653 | O1− |
| 1470 | C | GLU | 391 | −13.535 | −12.84 | 13.56 | C |
| 1471 | O | GLU | 391 | −14.497 | −13.128 | 14.308 | O |
| 1472 | OXT | GLU | 391 | −13.391 | −13.329 | 12.418 | O1− |
| 1473 | H | GLU | 391 | −13.501 | −10.247 | 14.883 | H |
| 1474 | HA | GLU | 391 | −11.631 | −11.983 | 13.337 | H |
| 1475 | HB2 | GLU | 391 | −11.124 | −11.618 | 15.763 | H |
| 1476 | HB3 | GLU | 391 | −12.709 | −12.27 | 16.157 | H |
| 1477 | HG2 | GLU | 391 | −11.272 | −14.031 | 16.525 | H |
| 1478 | HG3 | GLU | 391 | −11.964 | −14.429 | 14.955 | H |
| 1479 | C8 | MOL | 392 | −6.578 | 2.271 | 0.481 | C |
| 1480 | C6 | MOL | 392 | −5.466 | 1.518 | 0.847 | C |
| 1481 | S | MOL | 392 | −5.715 | −0.133 | 1.394 | S |
| 1482 | C3 | MOL | 392 | −4.098 | −0.81 | 1.481 | C |
| 1483 | C2 | MOL | 392 | −3.96 | −2.171 | 1.736 | C |
| 1484 | C1 | MOL | 392 | −2.688 | −2.745 | 1.888 | C |
| 1485 | N1 | MOL | 392 | −2.576 | −4.13 | 2.254 | N |
| 1486 | C12 | MOL | 392 | −3.569 | −5.055 | 1.658 | C |
| 1487 | C13 | MOL | 392 | −1.228 | −4.747 | 2.289 | C |
| 1488 | C | MOL | 392 | −1.552 | −1.945 | 1.713 | C |
| 1489 | C5 | MOL | 392 | −1.685 | −0.587 | 1.43 | C |
| 1490 | C4 | MOL | 392 | −2.947 | −0.018 | 1.323 | C |
| 1491 | N | MOL | 392 | −3.009 | 1.369 | 1.055 | N |
| 1492 | C7 | MOL | 392 | −4.186 | 2.093 | 0.757 | C |
| 1493 | C11 | MOL | 392 | −4.036 | 3.412 | 0.346 | C |
| 1494 | C10 | MOL | 392 | −5.15 | 4.157 | −0.034 | C |
| 1495 | C9 | MOL | 392 | −6.425 | 3.587 | 0.017 | C |
| 1496 | N2 | MOL | 392 | −7.541 | 4.346 | −0.45 | N |
| 1497 | C15 | MOL | 392 | −7.696 | 5.732 | 0.055 | C |
| 1498 | C14 | MOL | 392 | −8.867 | 3.704 | −0.53 | C |
| 1499 | H1 | MOL | 392 | −7.532 | 1.832 | 0.559 | H |
| 1500 | H3 | MOL | 392 | −4.829 | −2.76 | 1.839 | H |
| 1501 | H7 | MOL | 392 | −3.583 | −4.88 | 0.59 | H |
| 1502 | H8 | MOL | 392 | −4.533 | −4.861 | 2.106 | H |
| 1503 | H9 | MOL | 392 | −3.283 | −6.077 | 1.87 | H |
| 1504 | H10 | MOL | 392 | −1.32 | −5.764 | 2.647 | H |
| 1505 | H11 | MOL | 392 | −0.824 | −4.728 | 1.284 | H |
| 1506 | H12 | MOL | 392 | −0.617 | −4.181 | 2.978 | H |
| 1507 | H2 | MOL | 392 | −0.584 | −2.357 | 1.781 | H |
| 1508 | H4 | MOL | 392 | −0.824 | 0.018 | 1.292 | H |
| 1509 | H | MOL | 392 | −2.124 | 1.911 | 1.149 | H |
| 1510 | H6 | MOL | 392 | −3.07 | 3.845 | 0.31 | H |
| 1511 | H5 | MOL | 392 | −5.023 | 5.147 | −0.384 | H |
| 1512 | H16 | MOL | 392 | −6.737 | 6.227 | 0.099 | H |
| 1513 | H17 | MOL | 392 | −8.35 | 6.264 | −0.625 | H |
| 1514 | H18 | MOL | 392 | −8.144 | 5.672 | 1.036 | H |
| 1515 | H13 | MOL | 392 | −9.222 | 3.536 | 0.478 | H |
| 1516 | H14 | MOL | 392 | −9.53 | 4.37 | −1.067 | H |
| 1517 | H15 | MOL | 392 | −8.761 | 2.774 | −1.072 | H |

### Example 3—Structure-Based Design of Potent Tau Assembly Inhibitors

Overview

The molecular simulations performed in Example 2 suggest that LMT is able to bind dGAE and enhance the stabilisation of a compact folded conformation. The inventors hypothesized that molecules which are able to bind tightly within this pocket would further stabilise dGAE and prevent assembly into PHF. They therefore set out to analyse the pharmacophore features of the LMT binding pocket.

Methods

A pharmacophore model was designed by analysing the interactions between LMT and the LMT binding pocket identified in Example 2. A series of compounds were designed to test the binding hypothesis generated from this model. These compounds were tested in a cell-based tau aggregation assay as described in Rickard J E, Horsley D, Wischik C M, Harrington C R., Methods Mol Biol. 2017; 1523:129-140, using LMT (EC₅₀=0.096 μM) as a reference compound.

Results

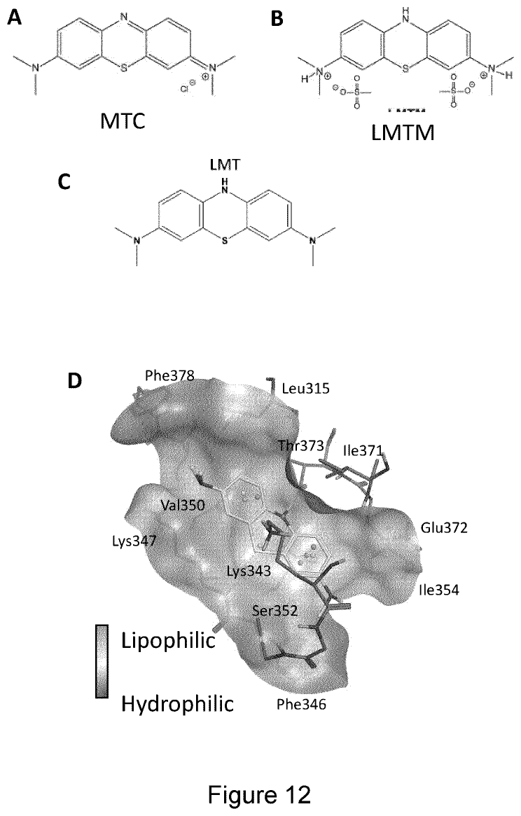

As shown on FIG. 12D, the LMT binding pocket is predominately hydrophobic in nature, with some sites suitable for forming hydrogen bonds. The site is capped by phenylalanine residues Phe378 and Phe346, and contains other hydrophobic side chains in Val350, Leu315, Ile354, Ile371. A number of side chains have the potential of forming hydrogen-bonds to a molecule within the pocket. These include the backbone of Lys347, the hydroxy group of Thr373, the backbone carbonyl of Leu315 and the NH of Glu372. LMT forms interactions with Lys347 and Thr373. LMT is also bound by Lys343, which has the potential to form Pi-cation interactions with the aromatic rings of LMT (see LMT structure, FIG. 12C).

Using in silico design approaches, a set of compounds were designed that exploit the hydrogen-bonding features observed with LMT, and the shape and lipophilic features of the LMT-induced binding pocket. These compounds were assessed in a cell-based Tau aggregation assay. The features of these compounds and the results of the cell-based Tau aggregation assays are shown in Table 2. All compounds had the same central ring and one of 6 substituents to the left of the central ring and one of 5 substituents to the right of the central ring (by comparison to the central ring, left and right rings or LMT shown on FIG. 12C). Compounds 15-18, 12-14, 9-11. 3-8 and 1-2, respectively, had the same substituent on the right. Compounds (3, 9, 15), (4, 10), (1, 5, 12, 16), (6, 13), (7, 14, 17) and (2, 8, 11, 18) respectively, had the same substituent on the left. The substituents were designed to test the required strength of hydrogen bonds with the pocket, as well as optimal orientation for hydrophobic and pi interactions. The results confirm the key features of the predicted stabilised cryptic pocket that can be exploited to inhibit Tau aggregation. Compound 17 showed the best activity with an EC₅₀=0.139 μM. This compound fulfilled a number of binding features, including hydrogen-bonding to the Glu372 backbone NH, hydrogen-bonding to the Lys374 backbone NH and a hydrogen bond to the NH of Thr373. Additionally, the compound comprises an aromatic substituent that binds in the lipophilic pocket towards Phe378, forming a face-edge Pi stack with Phe378, and it is well positioned to interact with the Lys343 through Pi-cation interactions.

The results suggest that interactions with Lys343 is important to differentiate compound potency, with both hydrogen bonding to the backbone carbonyl of Lys343 and Pi-cation interactions with Lys343 are favourable for ligand potency. Small substituents that are able to bind in the lipophilic pocket towards Phe378, and the ability to form a H-bond with the NH of Thr373 also appear relevant. The structural differences between compounds 17 and 18 include the removal of the aromatic substituent that binds in the lipophilic pocket towards Phe378, and the introduction of a substituent on one of the heavy atoms that formed a H bond with the NH of Thr373. These changes bring about a marked 16-fold loss in potency, compound 18 (EC₅₀=2.246 μM). The structural differences between compounds 17 and 14 include a reorientation of the right substituent (the left substituent in these compounds being identical), which affect the hydrogen-bonding orientation and hence strength between the left substituent of the ligand and the protein. The interaction with Phe378 is also lost. These changes result in a loss of potency by 8-fold (EC₅₀=1.17 μM for compound 14).

The structure-activity relationships of the 19 compounds examined (including LMT) supports the hypothesis that a stabilised cryptic pocket exists which can be exploited to achieve tau assembly inhibition.

TABLE 2
Compounds tested for tau aggregation inhibition

| | | Activity |
|---|---|---|
| Compound | Predicted interactions (average distance between heavy atoms) | (EC50, μM) |
| LMT | H-donor with THR373 sidechain OG1 (average distance | 0.096 |
| | between NH and OG1 of THR373 = 2.92) | |
| | H-acceptor with LYS347 backbone NH (average distance | |
| | between S and NH of LYS347 = 3.45) | |
| | pi-H with interaction SER352 backbone CA (average distance | |
| | between 6-ring and CA of SER352 = 4.1) | |
| | pi-cation interaction LYS343 sidechain NZ (average distance | |
| | between aromatic ring and NZ of LYS343 = 3.5) | |
| 17 | H-donor with GLU372 sidechain OE1 (average distance | 0.139 |
| | between H donor atom and OE1 of GLU372 = 3.52) | |
| | H-donor with LEU315 backbone C═O (average distance | |
| | between H donor atom and C═O of LEU315 = 3.17) | |
| | H-acceptor with ILE371 backbone CA (average distance | |
| | between H acceptor atom and CA of ILE371 = 3.25) | |
| | H-acceptor with SER341 sidechain CB (average distance | |
| | between H acceptor atom and CB of SER341 = 3.28) | |
| | H-acceptor with LYS343 backbone NH (average distance | |
| | between H acceptor atom and NH of LYS343 = 2.86) | |
| | H-acceptor with PHE346 backbone CA (average distance | |
| | between H acceptor atom and CA of PHE346 = 3.4) | |
| | H-acceptor with LYS347 backbone NH (average distance | |
| | between H acceptor atom and NH of LYS347 = 2.99) | |
| | pi-H with LYS343 sidechain CB (average distance between 5- | |
| | ring and CB of LYS343 = 4.21) | |
| | pi-cation with LYS343 sidechain NZ (average distance | |
| | between aromatic ring and NZ of LYS343 = 3.67) | |
| 12 | H-donor with GLU372 sidechain OE1 (average distance | 0.204 |
| | between H donor atom and OE1 of GLU372 = 3.07) | |
| | H-acceptor with ILE371 backbone CA (average distance | |
| | between H acceptor atom and CA of ILE371 = 3.24) | |
| | H-acceptor with GLU372 backbone NH (average distance | |
| | between H acceptor atom and NH of GLU372 = 2.98) | |
| | H-acceptor with LYS343 backbone NH (average distance | |
| | between H acceptor atom and NH of LYS343 = 3.42) | |
| | H-acceptor with LYS347 backbone NH (average distance | |
| | between H acceptor atom and NH of LYS347 = 3.02) | |
| 16 | H-acceptor with LYS347 backbone NH (average distance | 0.239 |
| | between H acceptor atom and NH of LYS347 = 3.14) | |
| | Hydrophobic interactions with the sidechains of Leu315, | |
| | Val350, Ile371 | |
| 1 | H-donor with GLU372 sidechain OE1 (average distance | 0.381 |
| | between H donor atom and OE1 of GLU372 = 2.97) | |
| | H-acceptor with ILE371 backbone CA (average distance | |
| | between H acceptor atom and CA of ILE371 = 3.32) | |
| | H-acceptor with GLU372 sidechain NH (average distance | |
| | between H acceptor atom and NH of GLU372 = 3) | |
| | H-acceptor with GLU342 backbone NH (average distance | |
| | between H acceptor atom and NH of GLU342 = 3.17) | |
| | H-acceptor with PHE346 backbone CA (average distance | |
| | between H acceptor atom and CA of PHE346 = 3.45) | |
| | H-acceptor with LYS347 backbone NH (average distance | |
| | between H acceptor atom and NH of LYS347 = 3.11) | |
| 7 | H-donor with LEU315 backbone C═O (average distance | 0.463 |
| | between H donor atom and C═O of LEU315 = 3.22) | |
| | H-acceptor with ILE371 backbone CA (average distance | |
| | between H acceptor atom and CA of ILE371 = 3.26) | |
| | H-acceptor with GLU372 backbone NH (average distance | |
| | between H acceptor atom and NH of GLU372 = 2.94) | |
| | H-acceptor with GLU342 backbone NH (average distance | |
| | between H acceptor atom and NH of GLU342 = 3.16) | |
| | H-acceptor with PHE346 backbone CA (average distance | |
| | between H acceptor atom and CA of PHE346 = 3.4) | |
| | H-acceptor with LYS347 backbone NH (average distance | |
| | between H acceptor atom and NH of LYS347 = 3.05) | |
| 3 | H-donor with LYS343 backbone C═O (average distance | 0.583 |
| | between H donor atom and C═O of LYS343 = 3.25) | |
| | H-donor with THR373 sidechain OG1 (average distance | |
| | between H donor atom and OG1 of THR373 = 3.12) | |
| 4 | H-donor with THR373 sidechain OG1 (average distance | 0.591 |
| | between H donor atom and OG1 of THR373 = 3.34) | |
| | H-acceptor with LYS369 backbone CE (average distance | |
| | between H acceptor atom and CE of LYS369 = 2.98) | |
| | H-acceptor with GLU372 backbone NH (average distance | |
| | between H acceptor atom and NH of GLU372 = 3.11) | |
| 9 | H-donor with THR373 sidechain OG1 (average distance | 0.682 |
| | between H donor atom and OG1 of THR373 = 3.04) | |
| | Hydrophobic interactions with Val350 and Leu315 and Ile371 | |
| 5 | H-acceptor with ILE371 backbone CA (average distance | 0.757 |
| | between H acceptor atom and CA of ILE371 = 3.28) | |
| | H-acceptor with GLU372 backbone NH (average distance | |
| | between H acceptor atom and NH of GLU372 = 2.98) | |
| | H-acceptor with GLU342 backbone NH (average distance | |
| | between H acceptor atom and NH of GLU342 = 3.14) | |
| | H-acceptor with LYS343 backbone NH (average distance | |
| | between H acceptor atom and NH of LYS343 = 3.47) | |
| | H-acceptor with PHE346 backbone CA (average distance | |
| | between H acceptor atom and CA of PHE346 = 3.4) | |
| | H-acceptor with LYS347 backbone NH (average distance | |
| | between H acceptor atom and NH of LYS347 = 3.09) | |
| 11 | pi-H with LYS343 sidechain CB (average distance between 5- | 0.926 |
| | ring and CB of LYS343 = 3.84) | |
| | Hydrophobic interactions with Val350 and Leu315 and Ile371 | |
| 14 | H-donor with GLU372 sidechain OE1 (average distance | 1.17 |
| | between H donor atom and OE1 of GLU372 = 2.87) | |
| | H-donor with LEU315 backbone C═O (average distance | |
| | between H donor atom and C═O of LEU315 = 3.13) | |
| | H-acceptor with LYS347 backbone NH (average distance | |
| | between H acceptor atom and NH of LYS347 = 3.14) | |
| | pi-H with LYS343 sidechain CB (average distance between 5- | |
| | ring and CB of LYS343 = 3.66) | |
| | pi-cation with LYS343 sidechain NZ (average distance between | |
| | aromatic ring and NZ of LYS343 = 3.63) | |
| 2 | H-donor with GLU372 sidechain OE1 (average distance | 1.397 |
| | between H donor atom and OE1 of GLU372 = 3.05) | |
| | H-acceptor with LYS343 backbone NH (average distance | |
| | between H acceptor atom and NH of LYS343 = 3.1) | |
| | H-acceptor with LYS347 backbone NH (average distance | |
| | between H acceptor atom and NH of LYS347 = 2.97) | |
| | pi-H with LYS343 sidechain CB (average distance between 5- | |
| | ring and CB of LYS343 = 4.04) | |
| 8 | H-acceptor with ILE371 backbone CA (average distance | 1.684 |
| | between H acceptor atom and CA of ILE371 = 3.27) | |
| | H-acceptor with GLU372 backbone NH (average distance | |
| | between H acceptor atom and NH of GLU372 = 2.96) | |
| | H-acceptor with GLU342 backbone NH (average distance | |
| | between H acceptor atom and NH of GLU342 = 3.14) | |
| | H-acceptor with PHE346 backbone CA (average distance | |
| | between H acceptor atom and CA of PHE346 = 3.35) | |
| | H-acceptor with LYS347 backbone NH (average distance | |
| | between H acceptor atom and NH of LYS347 = 3.09) | |
| 10 | H-donor with THR373 sidechain OG1 (average distance | 1.732 |
| | between H donor atom and OG1 of THR373 = 3.21) | |
| | H-acceptor with LYS369 backbone NZ (average distance | |
| | between H acceptor atom and NZ of LYS369 = 3.39) | |
| | pi-H with LYS343 backbone NH (average distance between | |
| | aromatic ring and NH of LYS343 = 4.44) | |
| | pi-H with LYS343 sidechain CB (average distance between 5- | |
| | ring and CB of LYS343 = 4.04) | |
| 6 | H-donor with LEU315 backbone C═O (average distance | 1.896 |
| | between H donor atom and C═O of LEU315 = 3.38) | |
| | H-acceptor with ILE371 backbone CA (average distance | |
| | between H acceptor atom and CA of ILE371 = 3.33) | |
| | H-acceptor with LYS343 backbone NH (average distance | |
| | between H acceptor atom and NH of LYS343 = 3.15) | |
| | pi-H with LYS343 sidechain CE (average distance between 6- | |
| | ring and CE of LYS343 = 3.7) | |
| 13 | H-acceptor with ILE371 backbone CA (average distance | 1.903 |
| | between H acceptor atom and CA of ILE371 = 3.26) | |
| | H-acceptor with GLU372 backbone NH (average distance | |
| | between H acceptor atom and NH of GLU372 = 2.96) | |
| | H-acceptor with LYS343 backbone NH (average distance | |
| | between H acceptor atom and NH of LYS343 = 3.24) | |
| | H-acceptor with PHE346 backbone CA (average distance | |
| | between H acceptor atom and CA of PHE346 = 3.3) | |
| | H-acceptor with LYS347 backbone NH (average distance | |
| | between H acceptor atom and NH of LYS347 = 3.01) | |
| 15 | H-donor with GLU372 sidechain OE1 (average distance | 1.981 |
| | between H donor atom and OE1 of GLU372 = 3.32) | |
| | H-donor with THR373 sidechain OG1 (average distance | |
| | between H donor atom and OG1 of THR373 = 3.01) | |
| 18 | H-donor with LYS343 backbone C═O (average distance | 2.246 |
| | between H donor atom and C═O of LYS343 = 3.57) | |
| | H-acceptor with PHE346 backbone CA (average distance | |
| | between H acceptor atom and CA of PHE346 = 3.46) | |
| | H-acceptor with LYS347 backbone NH (average distance | |
| | between H acceptor atom and NH of LYS347 = 3.21) | |

### Example 4—Mechanism for dGAE Aggregation into Paired Helical Filaments

Overview

Molecular dynamics simulation was used to study the assembly of dGAE in a conformation that is able to bind LMT, into a paired helical filament (PHF) arrangement.

Methods

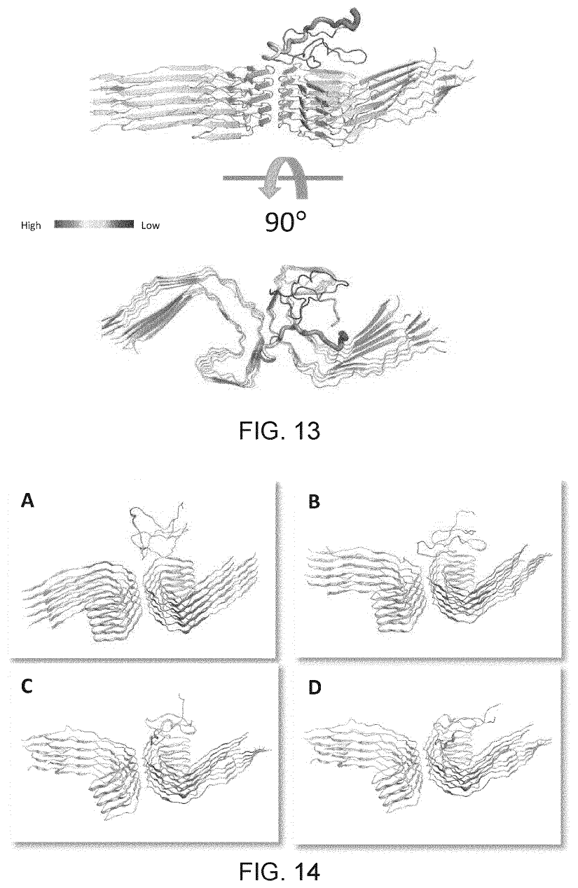

The parameterised 3D protein structures of the PHF 10 oligomer (stack of 10 monomers) modelled from PDB ID: 5O3L (as explained in Example 1), was used as a reference template to model the approach of a LMT-free dGAE monomer (as determined using Tau97 in Example 2) towards the PHF and then complete assembly. First the system was truncated from 97 to 73 residues according to the sequence reported in the literature (Fitzpatrick et al., 2017), which describes based on cryo-EM investigation of tau filaments that filament cores are made of two identical protofilaments comprising residues ³⁰⁶V-to-F³⁷⁸ of the Tau protein. This truncated system will be referred to as dGAE73 and PHF73 for convenience. The LMT-free dGAE73 monomer was obtained from the LMT MD simulation experiment (Example 2) and truncated appropriately. The C-terminal truncated amino acids were left as free acids and the N-terminal residues as free bases. Following an established protocol for MD simulations, system minimisation followed by an equilibration run, in total 1,200 ns, and a production MD simulation was then performed for 258 ns. A structure was extracted from the simulation at 50 ns which represented an average of the flexibility observed in the system over the 258 ns, see FIG. 13.

The 50 ns timeframe structure was allowed to assemble following the simulated annealing approach to nudged elastic band (NEB) method using the AMBER software. The 50 ns timeframe was used as a starting reference structure and the fully assembled and minimised state was used as a final reference structure. A total of 144 replica frames were employed in constructing the trajectory for assembly.

The NEB simulation was run in a number of stages, 1) Heating up the system, 2) simulated annealing and equilibration, and finally 3) slow cooling. These are described below.

During the heating stage, the system was allowed to warm up from 0 to 300K, performed with a small spring constant. A run of 20 ps of MD was performed with a 0.5 fs time step (nstlim=40000, dt=0.0005). The SHAKE algorithm was not used (ntc=1, ntf=1). Since the coordinates of the end points are fixed in Cartesian space in NEB and the intervening structures cannot move far due to the springs there is no need to re-centre the coordinates every few hundred steps so this option was turned off (NSCM=0). The generalised Born model was used for the implicit solvent with a sodium chloride salt concentration of 0.2M (igb=1, saltcon=0.2). Nonbonded cutoffs and truncated Born radii calculations were obtained by setting these to 999.0 angstroms (cut=999.0, rgbmax=999.0). The NEB specific options used were (ineb=1 [turn on neb], skmin=10, skmax=10 [spring constants]). The simulation was started with a relatively small spring constant of 10 KCal/mol/Å². For temperature regulation the langevin thermostat (ntt=3) was used together with a high collision rate of 1000 ps-1 (gamma_ln=1000). NMR weight restraints (nmropt=1) were used to linearly increase the value of temp0 from 0.0 to 300.0 K over 35,000 steps of the 40,000 step simulation (istep1=0, istep2=35000, value1=0.0, value2=300.0). This means that at step 0 the value of the target temperature (temp0) will be 0.0 K. At step 5,000 it will be 42.86 K, at 10,000 it will be 85.72 K, by 20,000 it will be 171.44 K and by 35,000 it will be at 300.0 K. It will then remain at 300.0 K until the end of the 40,000 step run.

In the simulated annealing and equilibration stage, a run of 100 ps of MD at 300K with a larger spring constant was used. The coordinates were saved at a frequency of once every 5,000 steps (ntwx=5000). The next step is to run a simulated annealing run. The NMR restraints option was used to control the value of temp0 during the run. A total of 300 ps of MD was run with the following temperature profile:
0-50 ps: Heat from 300 K to 400 K
50-100 ps: Equilibrate at 400 K
100-150 ps: Heat from 400 to 500 K
150-200 ps: Equilibrate at 500 K
200-250 ps: Cool to 300 K
250-300 ps: Equilibrate at 300 K

The final stage is to slowly cool the system down and this was performed in two stages. In the first stage the system was gradually cooled down in 50 K steps over 120 ps using the following profile:
0 to 10 ps:300K->250 K
10 to 20 ps: Equil at 250 K
20 to 30 ps: 250 K->200 K
30 to 40 ps: Equil at 200 K
40 to 50 ps: 200->150 K
50 to 60 ps: Equil at 150 K
60 to 70 ps: 150 K->100 K
70 to 80 ps: Equil at 100 K
80 to 90 ps: 100 K->50 K
90 to 100 ps: Equil at 50 K
100 to 110 ps:50 K->0 K
110 to 120 ps: Equil at 0 K

Finally, to calculate a final energy minimization of the path the velocity Verlet algorithm was used. This was performed in a one final 200 ps long stage of cooling where setting temp0=0.0 K completes the final quenched MD and having turned on the quenched velocity Verlet method (vv=1).

Results

In an effort to further evaluate the hypothetical LMT-stabilised conformation of dGAE, we used molecular dynamics simulations to study the assembly of PHFs from a conformer of dGAE which is able to bind LMT. A PHF arrangement of 5 stacked dGAE73 (assembled into a 10 monomers PHF, referred to herein as PHF73) oligomers and a monomer dGAE73 in the stabilised state were modelled in a fully solvated environment, as shown on FIG. 2, and in FIGS. 13 and 14A. The simulation was performed in two steps, as described in the Methods above. The first step was to search for the initial site of anchoring the monomer dGAE73 onto the PHF using traditional molecular dynamics simulations in explicit solvent. The second step was to examine the PHF formation using nudged elastic band (NEB) molecular dynamics simulations.

In the first stage (anchor stage), 13 molecular dynamics simulations were started and the orientation of alignment of the dGAE73 monomer relative to the PHF73 stack was observed. The starting orientation was randomly chosen, and the monomer was placed beyond hydrogen-bonding distance to the PHF73 stack. The dGAE73 monomer aligned itself over the residues Val337-Gly355 which form a tight hairpin (as shown on FIGS. 14A-14B) in a number of the simulations. This sequence of 19 residues contains numerous charged amino acids residues, including 4 acidic groups, 5 basic groups and 3 polar side chains. This result may be the result of long-range electrostatic forces pulling the monomer towards to the PHF stack. Once the hairpin (residues 337-355) is in contact with the PHF stack the hairpin (residues 337-355) tightly anchors itself. Over a simulation period of over 40 ns freely mobile N- and C-terminal arms of the dGAE73 monomer unravel from the tightly bound stable core, as is illustrated in FIGS. 13 and 14D.

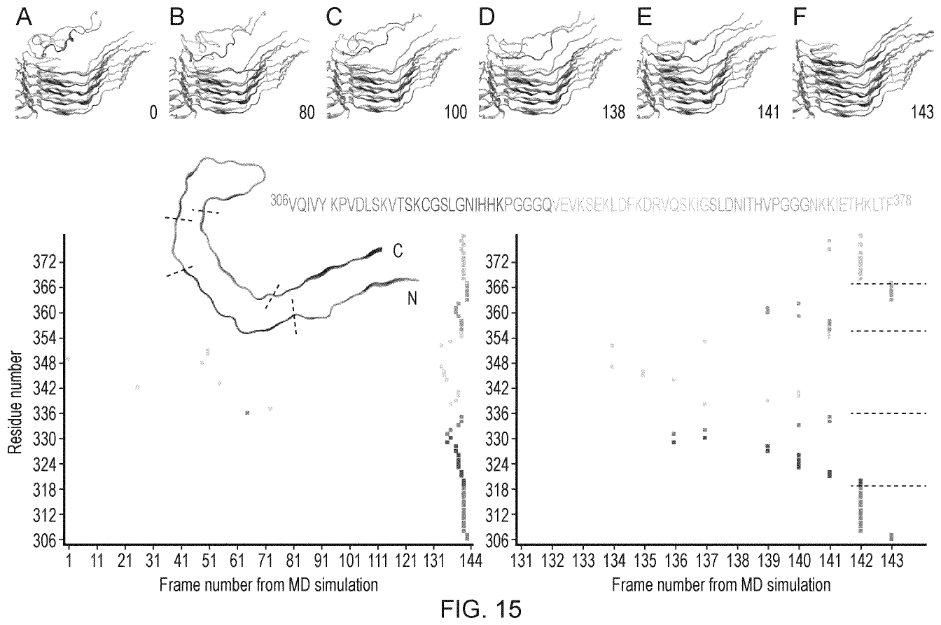

Then, after a period of 50 ns of production molecular dynamics simulation, a nudged elastic band (NEB) molecular dynamics simulation was used to find the minimum energy path for the rearrangement into the fully assembled PHF73 conformation. The observed folding pathway is shown on FIG. 15.

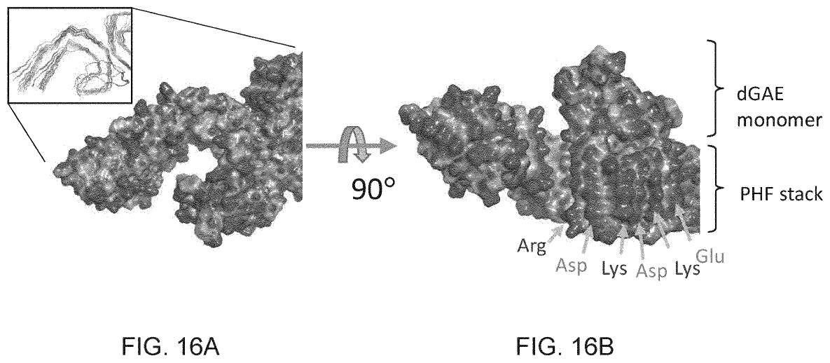

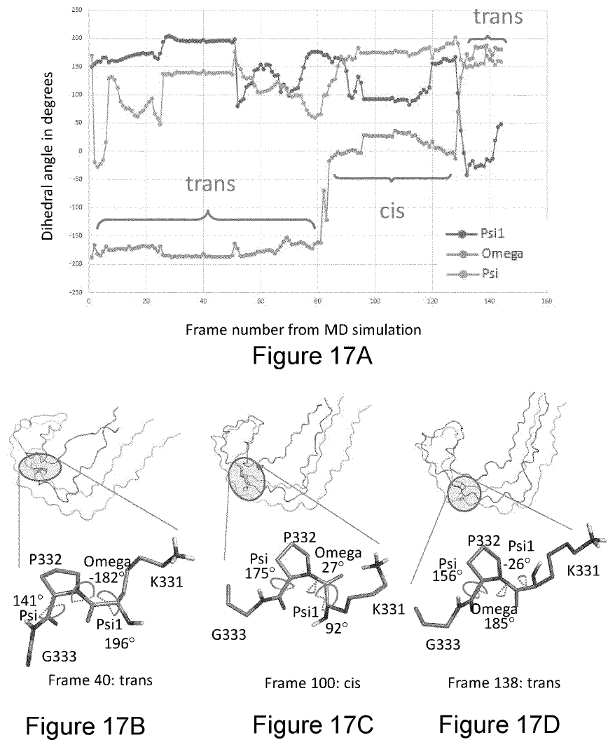

A key step in the formation of PHF73 is the formation of alternating positively charged and negatively charged sidechain stacks in the hairpin loop of PHF73 (residues Val337-Gln355), as best seen on FIG. 16B. This anchors the dGAE73 monomer to the PHF73. This is followed by another key step, the flipping of Pro332 from a trans to a cis configuration, at around frame 80, then back to a cis configuration, at around frame 130, as illustrated on FIG. 17. This enables the formation of interactions between His329, His330 and Lys331 of the monomer and the stack, zipping together from the C- to the N-terminal over a short period (see FIGS. 15 D-E). The N- and C-termini of the dGAE73 monomer then continue to unravel and the PHF73 formation completes in a zipper-like fashion, starting from Ile360-Thr361 (see FIGS. 15E-F). This occurs in two directions, first from 355-360 in the C- to N-direction, then from 361-367 in the N- to C-direction. Lastly, the cross-beta sheet conformation is formed, starting from Phe378 to Asn368 and closing the residues from C- to N-terminal, and joining residues 318-306 of the N-terminal in the C- to N-direction.

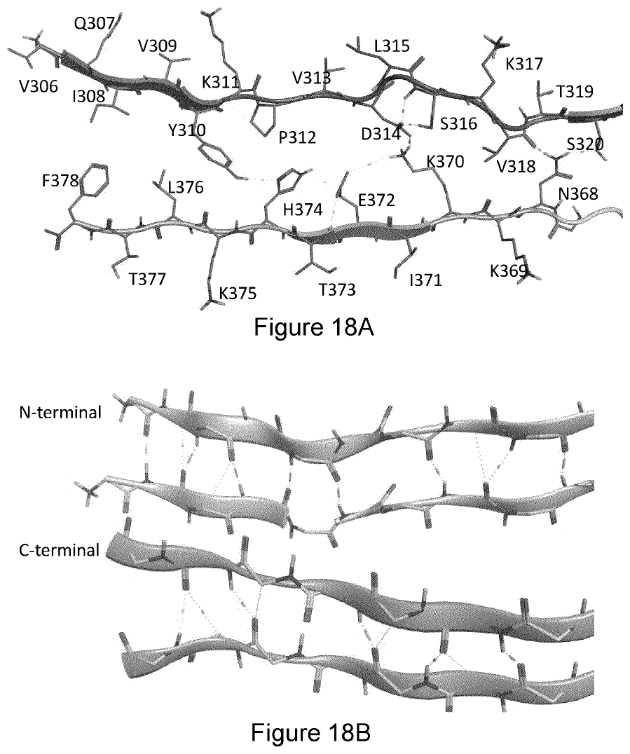

Through this process, the N-terminal arm (residues Val306-Lys321), closely coupled to the C-terminal arm (residues Gly367-Phe378), of dGAE73 collapses onto the PHF73 driven by electrostatic interactions between dGAE73 and the PHF73. Indeed, temporary hydrogen bonds between Asp314 in dGAE73 and Lys370 in the PHF73 stack and between Gln307 of dGAE73 and Lys375 and Thr377 of the PHF753 stack assist the zipper-like closure, helping to bring together the terminal arms of the dGAE73 and PHF73. During the zipper closure, the preferential binding conformation is maintained through hydrophobic interactions between the two arms (C- and N-ter) of the monomer. Indeed, the hydrophobic residues Ile308, Tyr310 and Pro312 of the N-terminal dGAE73 create well-packed hydrophobic interactions with the C-terminal residues of dGAE73 including Leu375 and Phe378, as shown on FIG. 18. The key residues mentioned above are highlighted in the sequence below (SEQ ID NO: 4 (dGAE73, human/mouse, 73 amino acids), together with indices showing the order of the steps mentioned above: [V₃₀₆QVYKPVDLSKV₃₁₈]⁶TSKCGSLGNI[H₃₂₉H₃₃₀K₃₃₁]³[P₃₃₂]²GGGQ[V₃₃₇EVKSEKLDFKDRVQ SKIG₃₅₅]¹ [SLDNI₃₆₀]⁴ [T₃₆₁HVPGGG₃₆₇]5[NKKIETHKLTF₃₇a]⁶

where the indices refer to:
Step 1: anchoring of monomer to PHF by formation of alternating positively charged and negatively charged sidechain stacks in the hairpin loop of PHF (residues Val337-Gln355).
Step 2: flipping of Pro332.
Step 3: formation of interactions between His329, His330 and Lys331 of the monomer and the stack, residues 319-331 zip together in a direction from C- to the N-terminal.
Step 4: zipping from 355-360 in the C- to N-direction.
Step 5: zipping from 361-367 in the N- to C-direction.
Step 6: formation of cross-beta sheet conformation, starting from Phe378 and closing the residues from C- to N-terminal, and joining residues 318-306 of the N-terminal in the C- to N-direction.

A crystal structure of a hexapeptide corresponding to residues V₃₀₆QIVYK₃₁₁ underlined in the above sequence was obtained by Sawaya M R, et al. (2007). This structure was used to design peptide inhibitors of VQIVYK aggregation by Sievers S A, et al. (2011) and Zheng J, et al. (2011). The PHF6 (306VQIVYK311) peptide has been shown to be sufficient for in vitro polymerization to filamentous structures and microcrystals (Goux et al., 2004; Sawaya et al., 2007; von Bergen et al., 2000 and 2001). The PHF6 motif is located in the repeat regions of the microtubule binding domain of tau and has been suggested to play a prominent role in the formation of PHFs and is also part of the PHF core composed of cross-β structure (47-48). Our observations support the finding that the PHF6 sequence in dGAE does form a cross-β structure, although it occurs at the end of the assembly stage and in our experiments is seen to be the last step in completing the stabilisation of the PHF, the closing of the zip.

More recently, the structure of another hexapeptide that is not part of dGAE (VQIINK) was determined and used to design inhibitors of aggregation based on the finding that VQIINK forms a more extensive steric zipper interface than VQIVYK. These inhibitors, peptides WINK and MINK, were found to reduce Tau40 aggregation.

Based on the modelling above, the peptide inhibitors of VQIVYK aggregation would at most be able to prevent the formation of the very last step of the aggregation process.

### Example 5—Examination of the Folding Pathway Using mAb Experiments

Overview

In this example, the inventors identified the core region of dGAE filaments (using protease digestion experiments) and monitored the progression of self-assembly of dGAE into filaments by following the surface exposure of specific epitopes to a range of monoclonal antibodies (using immuno-precipitation and ELISA experiments) were performed to examine the folding pathway and the effect that LMT had on the process.

Methods

Protein production: Recombinant tau297-391 (dGAE) was produced and purified from ‘E. coli as previously described in phosphate buffer (20 mM, pH 7.4) (AI-Hilaly et al, 2017) and stored at −20° C. until required.

Filament assembly: dGAE (100 μM) was incubated at 37° C. in phosphate buffer (20 mM, pH7.4), with or without DTT (10 mM) and, with or without agitation (700 rpm), for 24 hours.

Methylthioninium chloride (MTC): Methylthioninium chloride (MTC) was provided by TauRx Therapeutics Ltd. The concentrations are expressed as free methylthioninium base (MT), and MTC added as the specified time with or without DTT.

Protease digestion: 100 μM dGAE with 10 mM DTT in phosphate buffer (20 mM, pH 7.4), with or without MTC at ratios up to 1:5 was agitated at 700 rpm, 37° C. for 72 hours. 10 μg aliquots were digested with increasing concentrations of Proteinase K (PK), Pronase E (PE), or left untreated. The digestion was carried out at 37° C. for 1 hour then the reaction was stopped with 1 mM Pefabloc and samples were placed on ice for 5 minutes. Sample buffer without any reducing agent was added, incubated for 5 minutes at room temperature then loaded onto a gel.

Immunogold labelling transmission electron microscopy: For all dilutions of antibodies and secondary gold probes a modified phosphate buffer saline (PBS, pH 8.2) was used; this buffer was supplemented with BSA (1%), Tween-20 (0.005%), 10 mM Na EDTA, and NaN3 (0.2 g/I). dGAE-C322A (100 μM) was incubated with and without MT (10 μM) and incubated for 24 h at 37 C with agitation at 700 oscillations per min in dark. Preformed fibrils were decorated ‘on grid’ using a polyclonal anti-tau antibody (Sigma-Aldrich, SAB4501831). In summary, 4 μl of dGAE-C322 fibrils were pipetted onto Formvar/carbon coated 400-mesh copper TEM support grids (Agar Scientific, Essex, UK) and left for 1 min, then a filter paper was used to remove the excess. Normal goat serum (1.10 in PBS, pH 8.2) was used for blocking for 15 min at room temperature. Grids were then incubated with (10 μg/ml IgG) rabbit anti-tau polyclonal antibody for 2 h at room temperature, rinsed three times for two minutes using PBS (pH 8.2), and then immunolabeled in a 10-nm gold particle-conjugated goat anti-mouse IgG secondary probe (GaR10 British BioCell International, Cardiff, UK; 1.10 dilution) for 1 h at room temperature. The grids were then rinsed five times for two minutes in PBS (pH 8.2) and rinsed five times for two minutes in distilled water. Finally, the grids were negatively stained using 0.5% uranyl acetate. As a negative control, Aß342 fibrils were examined using the same protocol.

SDS-PAGE: SDS-PAGE was conducted on the entire assembly mixtures as well as the supernatant and pellet fractions used for CD (3 μl of each per lane). Samples were mixed with SDS-PAGE sample buffer (without reducing agent) and separated using Any kDa Mini-Protean™ TGX™ Precast gels (Bio-Rad) at 120 V, until the sample buffer reached the end of the gel. The gel was stained using Imperial Protein Stain (Thermo Scientific), following the manufacturer's instructions, before sealing the gel and scanning on a Canon ImageRunner Advance 6055i scanner.

Immunoblotting: A stock solution of recombinant dGAE was diluted to 100 μM in phosphate buffer (20 mM, pH7.4) with 10 mM DTT. The solution was agitated at 700 rpm, 37° C. and aliquots taken at t0 (following a 10 second vortex), 2, 4, 6, 8, 24 and 72 hours and stored at −20° C. When all the time points had been collected, samples were vortexed well and 3 μL of the whole mixture applied to a nitrocellulose membrane, 1.5 μL at a time, allow to almost dry then the next 1.5 μL applied. The membrane was washed for 2 minutes in TBS-T (tris buffered saline with 0.05% tween), 2 minutes in TBS then blocked in 5% milk in TBS-T for 1 hr. scAbs were diluted in block and incubated with the membrane for 1 hour followed by 3×10 minute washes in TBS-T. Secondary antibody (anti-Human GHRP, Invitrogen) diluted 1:1000 in block was then added to the membrane for 1 hour then washed 3×10 min TBS-T. ECL reagent (BioRad) was applied to the membrane for 3 minutes, drained then imaged using a scanner with a maximum exposure time of 10 minutes.

Results

dGAE Filaments Contain a Protease Resistant Core.

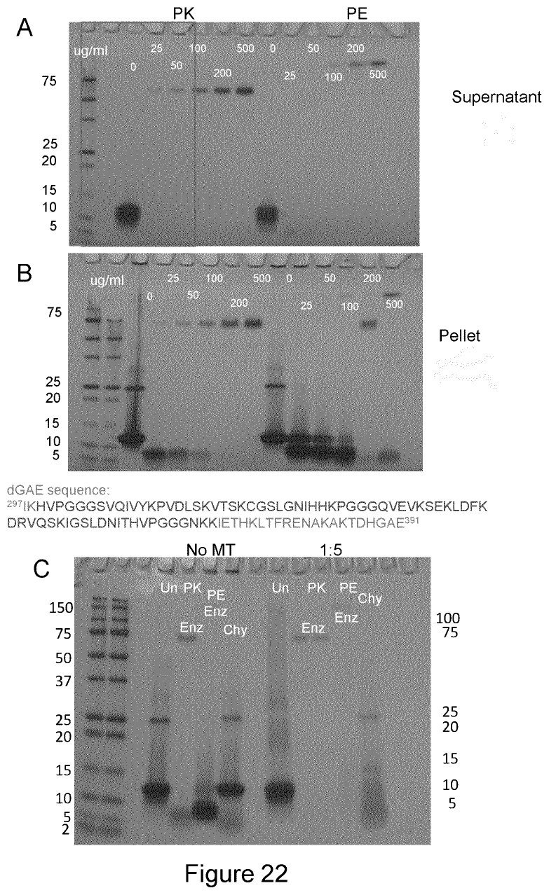

Soluble or fibrils of dGAE (100 μM) in reducing conditions (10 mM DTT) were incubated with increasing concentrations of proteinase K or Pronase E then samples were run on SDS-PAGE. Soluble dGAE runs as a doublet at 10/12Kda under reducing conditions and no bands of this size were observed in the presence of either PK or PE. Soluble dGAE in the supernatant was completely digested with even the lowest concentration of enzyme tested (25 μg/mL). The pellet contains mostly fibrillar dGAE and runs as a monomer ad dimer on SDS-PAGE. With PK, a protease resistant band at around 8 kDa was observed with enzyme concentrations up to around 100 μg/mL, after which the band disappeared indicating the core was fully digested. For PE the core withstood even up to 500 μg/mL enzyme. Mass spectrometry analysis of both protease resistant bands (see FIGS. 22A-B) revealed a protected core region of H299-K370 with a theoretical molecular weight of 7.5 kDa (data not shown).

Repeating these experiments in the presence of LMT revealed that LMT prevents the formation of a protease resistant core. Proteolysis revealed that soluble dGAE is digested while filaments retain a protease resistant core. In the absence of DTT, dGAE remains able to self-assemble and remains protease resistant (data not shown) while in the presence of DTT, LMT is able to prevent inhibition and the resulting soluble dGAE can be digested (FIG. 22C).

Assembly of dGAE Results in Progressive Loss of Epitopes

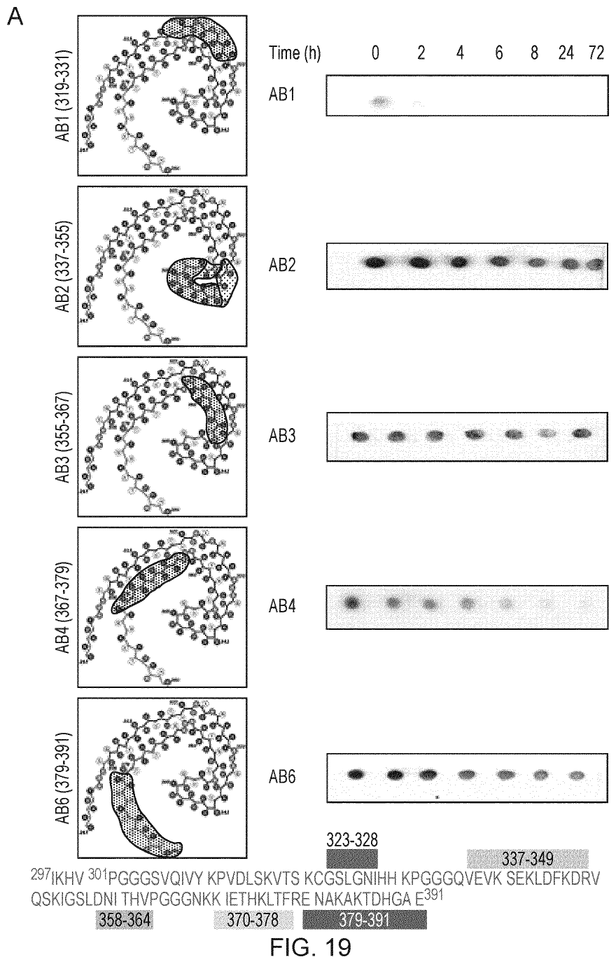

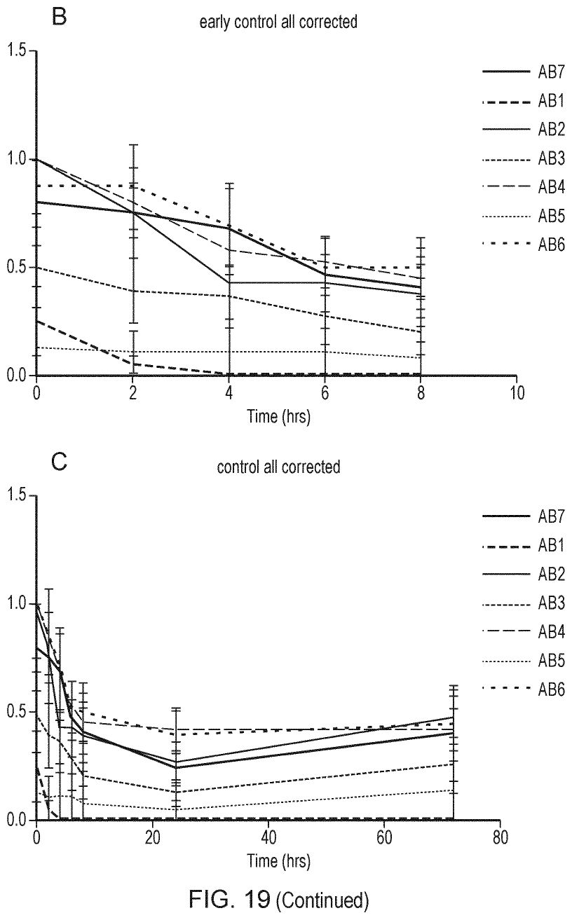

A series of monoclonal single chain antibodies (scAbs) raised to recognise different regions of the peptide were used to perform epitope mapping to follow the conformational change that accompanies self-assembly. Immunoblots were performed on 100 uM dGAe with 10 mM DTT dGAE assembled in reducing conditions over 72 hours at different time points. These were repeated 10 times and the data was pooled to produce graphical output normalised to the intensity at time 0 and error bars were provided to show the variation in the data (see FIG. 19). In particular, dGAE (100 μM, 20 mM phosphate buffer pH7.4 with 10 mM DTT) was agitated at 37° C. for 72 hours in the absence or presence of LMT (reduced) (1:5 ratio). Samples were withdrawn at 2, 4, 6, 8, 10, 20, 40 and 60 hours of agitation and 3 μL placed on a nitrocellulose membrane. Membrane was rinsed with TBS-T and incubated with antibodies binding to residues 319-331 (AB1), 337-355 (AB2), 355-367 (AB3), 367-378 (AB4), 379-391 (AB5), 379-390 (AB6), and 306-359 (AB7) for 1 hour followed by HRP-conjugated anti-sheep antibody for 1 hour and washed again washed with TBS-T x5. Detection was performed using Clarity ECL blotting substrate (BioRad) and exposed to X-ray film. The results of these experiments are shown on FIG. 19, for the experiments without LMT. The epitopes bound by the antibodies AB1-AB6 are highlighted in the sequence below (residues 3 to 97 of SEQ ID NO: 4; also referred to herein as dGAE, SEQ ID NO: 3, human/mouse, 95 amino acids)—with indices referencing the antibody corresponding to the epitope in bold between brackets. The sequence below also shows the sequence of dGAE underlined.

I₂₉₇HVPGGGSVQIVYKPVDLSKV[T₃₁₉SKCGSLGNIHHK₃₃₁]^{AB1}PGGGQ[V₃₃₇EVKSEKLDFKDRV QSKI{G₃₅₅]^{AB2}SLDNITHVPGG[G₃₆₇}^{AB3}NKKIETHKLTF₃₇₈]^{AB4}[{R₃₇₉ENAKAKTDHGA₃₉₀}^{AB6}E₃₉₁]^{AB5}

Binding Profiles in the Absence of LMT

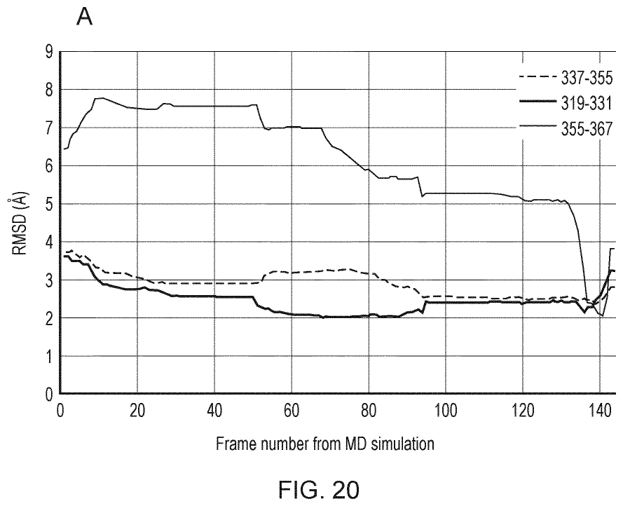

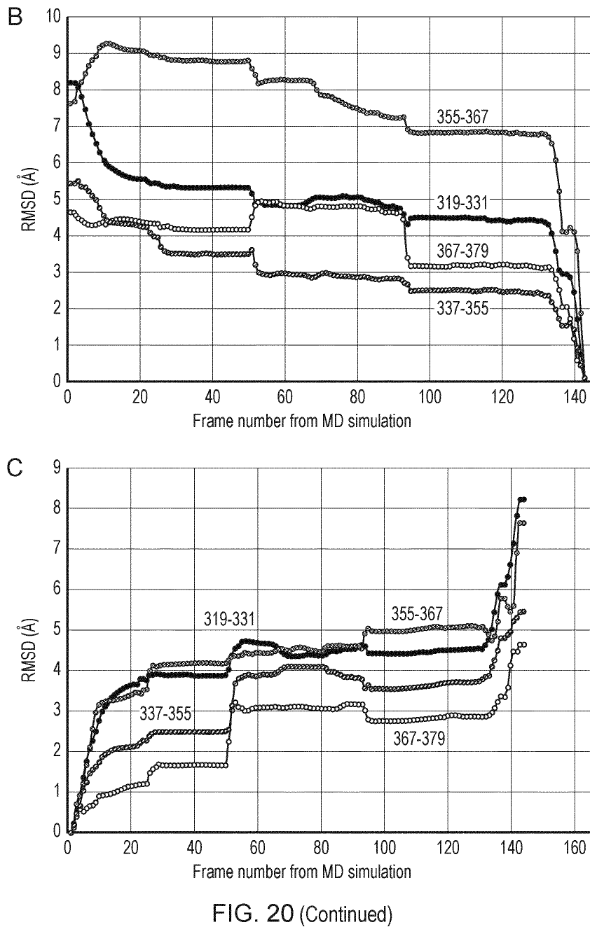

As best seen on FIG. 19B, at time 0 (before agitation), the antibodies AB2 (337-355), AB4 (367-378) and AB5 (379-391) are able to bind dGAE, indicating that their epitopes are exposed. The epitope of AB3 (355-367) appears to be less exposed. In order to elucidate this, the results of the NEB simulation described in Example 4 were used to compute the distance between the conformation of the epitopes AB1 to AB4 as they progress through the aggregation process, and the conformation of these epitopes at the start or the end of the aggregation process. The results of this analysis are shown on FIGS. 20B-C. This analysis revealed that in the stable free dGAE conformation (starting point of the simulations in Example 4), the epitope of AB3 (355-367) is partially occluded by residues 321-343. Further, this analysis revealed that the epitope of AB3 initially moves further away from the final bound conformation in the first 10 frames of the simulation, then “jumps” to a conformation close to that found in the fully assembled PHF around frame 133. This is in marked contrast with the behaviour of the epitopes of AB2 (337-355) and AB4 (367-378), which do not show this initial behaviour in the first 10 frames of the simulation. The conformations close to that in the fully assembled PHF are expected to enable slightly more binding of AB3 (355-367), which could explain the increase in signal in the dot blot experiments from 20 to 70 hours (see FIG. 19C).

Turning now to AB1 (319-331), the signal from the Dot Blot experiments implies that this epitope conformation is less exposed at t0 than AB2-5 (see FIG. 19B). This antibody recognises 323-328, which is found at the PHF-dimer interface and shows low affinity for the soluble protein at time 0 h and this is lost by 2 h. As shown on FIG. 20B, the conformation of this epitope changes rapidly (in the first 10 frames) during assembly, which is reflected in a rapid loss of signal in the Dot Blot experiments.

Turning to AB2 (337-355), in the context of PHF structure (Fitzpatrick, 2017) is found at the b-helix (see FIG. 19A). As the protein folds into the PHF structure, recognition of this region reduces gradually but the Dot Blot data indicates that this epitope may be in a conformation suitable for binding of the antibody throughout assembly (see FIG. 19B-C). The simulation data is in accordance with this, since the conformation of this epitope is within 3 Å of the final fully bound conformation for a significant part of the assembly (see frames 50 onwards on FIG. 20B).

The short AB4 (367-378, which binds within the C-shape region of the PHF, see FIG. 19A) epitope sequence appears to be bind to the antibody effectively throughout PHF assembly (see FIG. 19B-C, where the signal only gets as low as 0.5). Analysis of the RMSD of the AB4 sequence in dGAE during the assembly does not show a marked change in RMSD, the maximum RMSD is about 4 Å with an average close to 2 Å (see FIG. 20B). Further, the AB4 sequence is close to the C-terminal flexible jaws of the PHF. This inherent flexibility may allow detection by the antibody, albeit not optimally, throughout the PHF assembly. This flexibility of the C-terminal is reflected in the similar responses in the epitopes AB7 (306-359) and AB5 (379-390), see FIG. 19.

Together with the data from Example 4, this data confirms that the epitope of AB2 (337-355) is first to bind to PHF, from the middle of the sequence at R349. This is followed by the binding of charged groups E342, D348, followed by the sequences of V350-Q351-K343, Q336-V337-K347. The PGGG repeat between the epitopes of AB1 (319-331) and AB2 (337-355) then binds, followed by residues 1360-T261 in the epitope of AB3 (355-367). Then slowly the C-terminal end of AB1 (319-331) binds sequentially towards to N-terminal end, and the epitope of AB4 (367-378) then binds to PHF. This starts with starts with K375 and then amino acids on both sides are assembled. The final sequences to bind is the P364-GG-G367 sequence in the epitope of AB3 (355-378).

From these experiments, we can infer that as dGAE follows a sequence whereby the association of two molecules initiates the assembly (AB1, 323-328) followed by a folding into a C-shape (AB3, 358-364) and the formation of the b-helical hairpin. The structure then curves to bury (AB4, 370-378) and (AB6, 379-391) regions at the centre and C-termini.

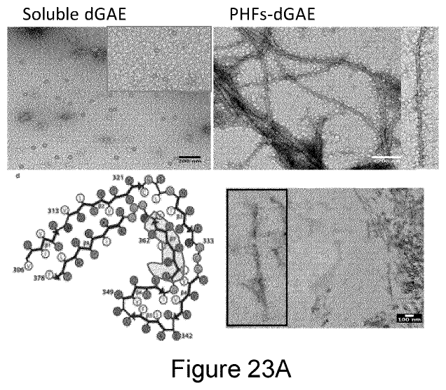

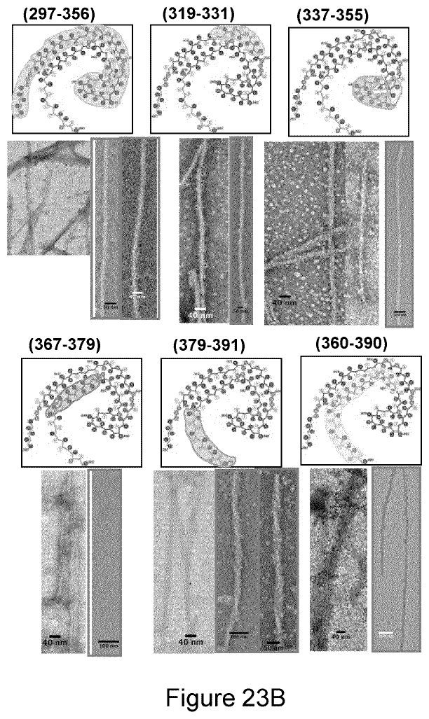

Immunogold labelling of soluble and preformed filaments of dGAE confirmed that the AB3 recognition sequence is exposed in soluble dGAE and buried in PHF (see FIG. 23A). In fact, none of the antibodies are able to access dGAE PHF (see FIG. 23B). Furthermore, AB3 does not recognise PHF in AD human brain tissue.

LMT Mediated Inhibition of dGAE Aggregates and Restoration of Immunoreactivity

The truncated core repeat domain dGAE (297-391), is the predominant fragment that constitutes bulk of the PHF core in AD (Wischik et al, 1988). During dGAE aggregation in vitro, scAb binding regions on dGAE are ‘hidden’ or ‘occluded’ which leads to a loss of immunoreactivity in aggregation samples. Here we have shown the occlusion of binding regions in aggregated dGAE samples and the recovery of immunoreactivity in the presence of LMTM, a tau-aggregation inhibitor. The scAbs tested for binding are core region specific with binding regions given in Table 3 below). For preparing the aggregates, 10 μl 10 mM DTT was added to 1000 μl 100 μM dGAE and agitated with/without LMTM (1:5 ratio) at 700 rpm for 24 h at 37° C. Following overnight agitation, one third of each sample was kept aside as ‘total’ and the rest was spun down at 16000×g for 30 min and separated into ‘supernatant’ and ‘pellet’. The pellet was then resuspended in half the original volume for further experiments. The immunoreactivities of core region specific scAbs towards dGAE aggregates formed with/without LMTM was tested using a sandwich ELISA format using a ‘E’ specific monoclonal antibody 423 mAb. This mAb has been shown to specifically bind to the Pronase resistant core structure in the PHFs (Wischik et al, 1988). ELISA plates were coated with 10 μg/ml 423 mAb and blocked as normal. Doubling dilutions of dGAE aggregate ‘total’, ‘supernatant’ and ‘pellet’ samples at 10 μg/ml starting concentration were added to designated wells in doubling dilutions in 1×PBS. dGAE monomer (non-aggregated) was included as assay control. All double dilutions were done in final volumes of 100 μl. This was left to incubate on lab bench for 1 h followed by the addition of test scAbs at 10 μg/ml. Anti-HuCk HRP labelled secondary antibody was added as described previously and ELISA data generated is represented using the graph below.

TABLE 3
Core binding scAbs tested in epitope occlusion assays
and their specific binding regions on Ht40

| | Binding regions |
|---|---|
| scAbs tested | on hT40 |
| AB2 | 337-355 |
| AB3 | 355-367 |
| AB5 | 360-390 |
| AB1 | 319-331 |
| AB7 | 297-356 |
| AB8 | 367-379 |

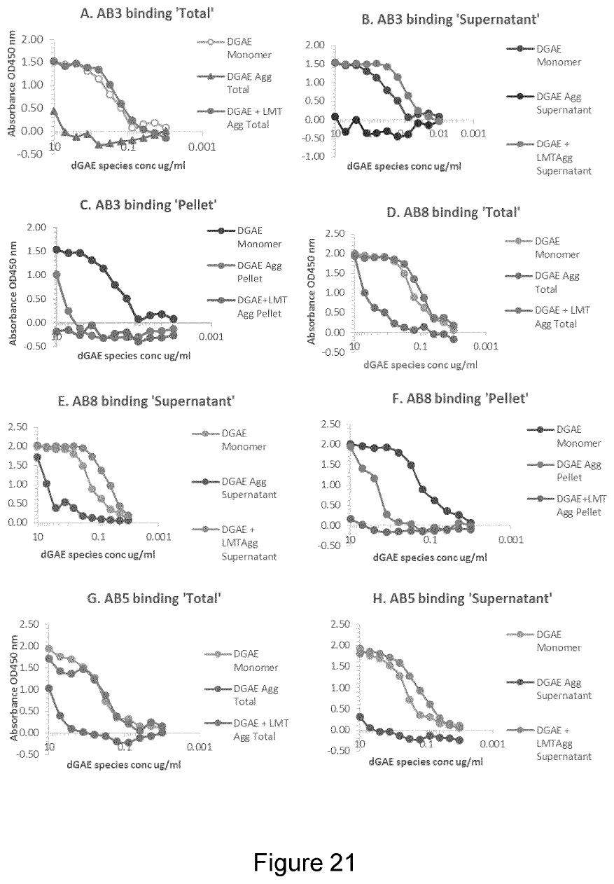

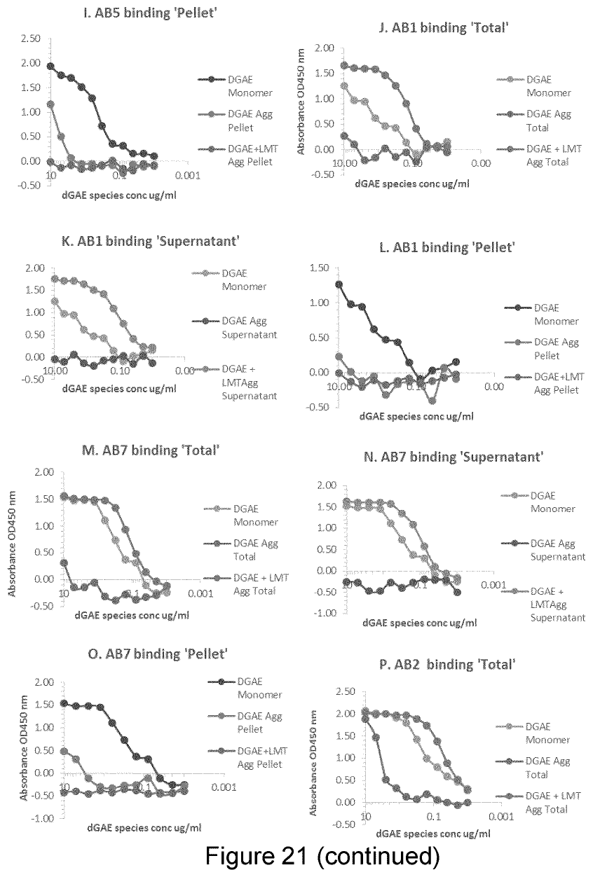

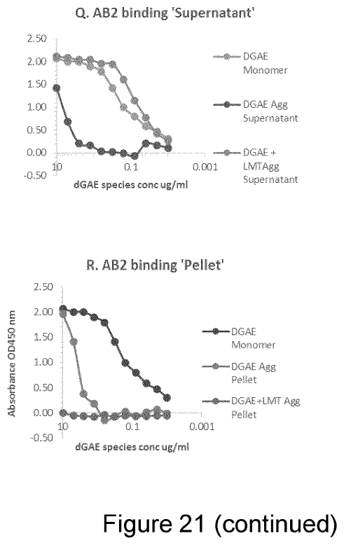

All scAbs tested showed increased binding to aggregated dGAE ‘total’ and ‘supernatant’ samples, when aggregation was conducted in the presence of LMTM. This proves the opening or revealing of occluded antibody binding regions on dGAE where LMTM is preventing the aggregation event, leading to an increased immunoreactivity (FIG. 21).

### REFERENCES

Youssra K. AI-Hilaly Bronwen E. Foster Luca Biasetti Liisa Lutter Saskia J. Pollack Janet E. Rickard John M. D. Storey Charles R. Harrington Wei-Feng Xue Claude M. Wischik Louise C. Serpell. Tau (297-391) forms filaments that structurally mimic the core of paired helical filaments in Alzheimer's disease brain. FEBS LettersVolume 594, Issue 5 (2020).
Al-Hilaly Y K, Pollack S J, Vadukul D M, Citossi F, Rickard J E, Simpson M, Storey J M D, Harrington C R, Wischik C M and Serpell L C (2017) Alzheimer's disease-like paired helical filament assembly from truncated tau protein is independent of disulfide crosslinking. J Mol Biol 429, 3650-3665.
Youssra K A I-Hilaly, Saskia J Pollack, Janet E Rickard, Michael Simpson, Ana-Caroline Raulin, Thomas Baddeley, Pascale Schellenberger, John M D Storey, Charles R Harrington, Claude M Wischik, Louise C Serpell. Cysteine-independent inhibition of Alzheimer's disease-like paired helical filament assembly by leuco-methylthioninium (LMT). Journal of molecular biology, 430:21, p. 4119-4131 (2018).
Ardita Shkurti, Ramon Goni, Pau Andrio, Elena Breitmoser, lain Bethune, Modesto Orozco, Charles A. Laughton; pyPcazip: A PCA-based toolkit for compression and analysis of molecular simulation data; SoftwareX, Volume 5, 2016, Pages 44-50.
P. W. Arriagada, J. H. Growdon, E. T. Hedley-White, B. T. Hyman, Neurofibrillary tangles but not senile plaques parallel duration and severity of Alzheimer's disease, Neurology 42 (1992) 631-639.
C. Bancher, K. Jellinger, H. Lassmann, P. Fischer, F. Leblhuber, Correlations betweenmental state and quantitative neuropathology in the Vienna Longitudinal Study on Dementia, Eur. Arch. Psychiatry Clin. Neurosci. 246 (1996) 137-146.
C. Bancher, H. Braak, P. Fischer, K. Jellinger, Neuropathological staging of Alzheimer lesions and intellectual status in Alzheimer's and Parkinson's disease, Neurosci. Lett. 162 (1993) 179-182.
Bouras C., Buée L., Delacourte A., Perl D. P and Morrison J. H. (1992) Differential distribution of neurofibrillary tangles in the cerebral cortex of dementia pugilistica and Alzheimer's disease cases. Acta Neuropathol. 85, 23-30.
Braak, H., Del Tredici, K, Braak, E. (2003) Spectrum of pathology. In Mild cognitive impairment: Aging to Alzheimer's disease edited by Petersen, R. C.; pp. 149-189.
M. Bucciantini, E. Giannoni, F. Chiti, F. Baroni, L. Formigli, J. Zurdo, N. Taddei, G. Ramponi, C. M. Dobson and M. Stefani, Nature, 2002, 416, 507-511.
Bucciantini, G. Calloni, F. Chiti, L. Formigli, D. Nosi, C. M. Dobson and M. Stefani, J. Biol. Chem., 2004, 279, 31 374-31 382.
Buee, L., Bussiere, T., Buee-Scherrer, V., Delacourte, A. & Hof, P. R. Tau protein isoforms, phosphorylation and role in neurodegenerative disorders. Brain Res. Rev. 33, 95-130 (2000).
S. Campioni, B. Mannini, M. Zampagni, A. Pensalfini, C. Parrini, E. Evangelisti, A. Relini, M. Stefani, C. M. Dobson, C. Cecchi and F. Chiti, Nat. Chem. Biol., 2010, 6, 140-147.
D. A. Case, D. S. Cerutti, T. E. Cheatham, Ill, T. A. Darden, R. E. Duke, T. J. Giese, H. Gohlke, A. W. Goetz, D. Greene, N. Homeyer, S. Izadi, A. Kovalenko, T. S. Lee, S. LeGrand, P. Li, C. Lin, J. Liu, T. Luchko, R. Luo, D. Mermelstein, K. M. Merz, G. Monard, H. Nguyen, I. Omelyan, A. Onufriev, F. Pan, R. Qi, D. R. Roe, A. Roitberg, C. Sagui, C. L. Simmerling, W. M. Botello-Smith, J. Swails, R. C. Walker, J. Wang, R. M. Wolf, X. Wu, L. Xiao, D. M. York and P. A. Kollman (2017), AMBER 2017, University of California, San Francisco.
Charpiot, A., Delacourte A., Buee, L., Purohit, D., Perl D. P. and Bouras, C. (1992) Distribution of neurofibrillary tangles and senile plaques in the cerebral cortex in postencephalitic parkinsonism. Neurosci. Lett. 139, 10-14
D. T. Chien, S. Bahri, A. K. Szardenings, J. C. Walsh, F. Mu, M.-Y. Su, et al., Early clinical PET imaging results with the novel PHF-tau radioligand [F-18]-T807, J. Alzheimers Dis. 34 (2013) 457-468.
F. Chiti and C. M. Dobson, Annu. Rev. Biochem., 2006, 75, 333-366.
Crowley M, Darden T, Cheatham T, Deerfield D: Adventures in Improving the Scaling and Accuracy of a Parallel Molecular Dynamics Program. The Journal of Supercomputing 1997, 11:255-278.
Darden T, York D, Pedersen L: Particle mesh Ewald-—an Nlog(N) method for Ewald sums in large systems. J Chem Phys 1998, 98:10089-10092.
C. Duyckaerts, M. Bennecib, Y. Grignon, T. Uchihara, Y. He, F. Piette, et al., Modeling the relation between neurofibrillary tangles and intellectual status, Neurobiol. Aging 18 (1997) 267-273.
Fitzpatrick, A. W. P., Falcon, B., He, S., Murzin, A. G., Murshudov, G., Garringer, H. J., Crowther, R. A., Ghetti, B., Goedert, M., Scheres, S. H. W. (2017) Nature 547: 185-190
Flament S., Delacourte A. and Mann D. M. A. (1990) Phosphorylation of tau proteins: a major event during the process of neurofibrillary degeneration. A comparative study between A D and Down's syndrome. Brain Res., 516, 15-19.
Gerson J E, Mudher A, Kayed R. Potential mechanisms and implications for the formation of tau oligomeric strains. Crit Rev Biochem Mol Biol. 2016:1-15.
C. G. Glabe, Structural Classification of Toxic Amyloid Oligomers. J. Biol. Chem., 2008, 283, 29 639-29 643.
Goedert, M., et al., 1989, Cloning and sequencing of the cDNA encoding an isoform of microtubule-associated protein tau containing four tandem repeats: differential expression of tau protein mRNAs in human brain. EMBO J., Vol. 8, pp. 393-399.
Goedert, M., et al., 1989, Multiple isoforms of human microtubule-associated protein tau: sequences and localization in neurofibrillary tangles of Alzheimer's disease. Neuron, Vol. 3, pp. 519-526.
Goux W J, Kopplin L, Nguyen A D, Leak K, Rutkofsky M, et al. (2004) The formation of straight and twisted filaments from short tau peptides. J Biol Chem 279: 26868-26875.
E. Grober, D. Dickson, M. J. Sliwinski, H. Buschke, M. Katz, H. Crystal, et al., Memory and mental status correlates of modified Braak staging, Neurobiol. Aging 20 (1999) 573-579.
Guest, M. F.; Bush, I. J.; Van Dam, H. J. J.; Sherwood, P.; Thomas, J. M. H.; Van Lenthe, J. H.; Havenith, R. W. A.; Kendrick, J. The GAMESS-U K electronic structure package: algorithms, developments and applications. Mol. Phys. 2005, 103 (6-8), 719-747.
C. R. Harrington, J. M. D. Storey, S. Clunas, K. A. Harrington, D. Horsley, A. Ishaq, et al., Cellular models of aggregation dependent template-directed proteolysis to characterize tau aggregation inhibitors for treatment of Alzheimer's disease, J. Biol. Chem. 290 (2015) 10862-10875.
Harrington, C. R., Perry, R. H., Perry, E. K., Hurt, J., McKeith, I. G., Roth, M. & Wischik, C. M. (1994) Senile dementia of Lewy body type and Alzheimer type are biochemically distinct in terms of paired helical filaments and hyperphosphorylated tau protein. Dementia 5, 215-228.
Ikeda K., Akiyama H., Kondo H., Arai T., Arai N. and Yagishita S. (1995) Numerous glial fibrillary tangles in oligodendroglia in cases of Subacute sclerosing panencephalitis with neurofibrillary tangles. Neurosci. Lett., 194, 133-135
R. Jakes, M. Novak, M. Davison, C. M. Wischik, Identification of 3- and 4-repeat tau isoforms within the PHF in Alzheimer's disease, EMBO J. 10 (1991) 2725-2729.
Jorgensen W: Revised TIPS for simulations of liquid water and aqueous solutions. J Chem Phys 1982, 77:4156-4163.
Jorgensen W L, Chandrasekhar J, Madura J D, Impey R W, Klein M L: Comparison of simple potential functions for simulating liquid water. J Chem Phys 1983, 79:926-935.
R. Kayed, E. Head, J. L. Thompson, T. M. McIntire, S. C. Milton, C. W. Cotman and C. G. Glabe, Science, 2003, 300, 486-489.
J. W. Kelly, Mechanisms of amyloidogenesis. Nat. Struct. Mol. Biol., 2000, 7, 824-826.
R. Kodali and R. Wetzel, Polymorphism in the Intermediates and Products of Amyloid Assembly. Curr. Opin. Struct. Biol., 2007, 17, 48-57.
Lasagna-Reeves C A, Castillo-Carranza D L, Sengupta U, Sarmiento J, Troncoso J, Jackson G R, et al. Identification of oligomers at early stages of tau aggregation in Alzheimer's disease. FASEB Journal, 2012; 26:1946-59.
Lee, V. M., Goedert, M. & Trojanowski, J. Q. Neurodegenerative tauopathies. Annu. Rev. Neurosci. 24, 1121-1159 (2001).
Love, S., Bridges, L. R. & Case, C. P (1995), Neurofibrillary Tangles in Niemann-Pick Disease Type C. Brain, 118, 119-129.
Macdonald J A, Bronner I F, Drynan L, Fan J, Curry A, Fraser G, et al. Assembly of transgenic human P301S Tau is necessary for neurodegeneration in murine spinal cord. Acta neuropathologica communications. 2019; 7:44.
James A. Maier, Carmenza Martinez, Koushik Kasavajhala, Lauren Wickstrom, Kevin E. Hauser and Carlos Simmerling: ff14S B: Improving the Accuracy of Protein Side Chain and Backbone Parameters from ff99S B. J. Chem. Theory Comput. 2015, 11, 8, 3696-3713.
M. Maruyama, H. Shimada, T. Suhara, H. Shinotoh, B. Ji, J. Maeda, et al., Imaging of tau pathology in a tauopathy mouse model and in Alzheimer patients compared to normal controls, Neuron 79 (2013) 1094-1108.
Maurage, C. A., Udd, B., Ruchoux, M. M., et al. (2005) Neurology, 65, 1636-1638
McKee, A., Cantu, R., Nowinski, C., Hedley-Whyte, E., Gavett, B., Budson, A., Santini, V., Lee, H.-S., Kubilus, C. & Stern, R. (2009) Chronic traumatic encephalopathy in athletes: progressive tauopathy after repetitive head injury. Journal of Neuropathology & Experimental Neurology 68, 709-735.
Meisl G, Kirkegaard J B, Arosio P, Michaels T C, Vendruscolo M, Dobson C M, et al. Molecular mechanisms of protein aggregation from global fitting of kinetic models. Nat Protoc. 2016; 11:252-72.
V. Melis, M. Magbagbeolu, J. E. Rickard, D. Horsley, K. Davidson, K. A. Harrington, et al., Effects of oxidized and reduced forms of methylthioninium in two transgenic mouse tauopathy models, Behav. Pharmacol. 26 (2015) 353-368.
Meyer T, Ferrer-Costa C, Perez A, Rueda M, Bidon-Chanal A, Luque F J, Laughton C A, Orozco M: Essential Dynamics: A Tool for Efficient Trajectory Compression and Management. J Chem Theory Comput 2006, 2:251-258.
MOE (The Molecular Operating Environment) Version 2016.10, software available from Chemical Computing Group Inc., 1010 Sherbrooke Street West, Suite 910, Montreal, Canada H3A 2R7. http://www.chemcomp.com
Modler A J, Fabian H, Sokolowski F, Lutsch G, Gast K, et al. (2004). Amyloid: J Protein Folding Disord 11: 215-231.
E. B. Mukaetova-Ladinska, F. Garcia-Sierra, J. Hurt, H. J. Gertz, J. H. Xuereb, R. Hills, et al., Staging of cytoskeletal and β-amyloid changes in human isocortex reveals biphasic synaptic protein response during progression of Alzheimer's disease, Am. J. Pathol. 157 (2000) 623-636.
Novak, M., Jakes, R., Edwards, P., Klug, A., Milstein, C. and Wischik, C. PNAS, 88, 5837-5841 (1991).
Novak, M., Kabat, J. and Wischik, C. M. Molecular characterization of the minimal protease resistant tau unit of the Alzheimer's disease paired helical filament. The EMBO Journal vol. 12 no. 1pp.365-370, (1993).
Novo, M., Freire, S. & A I-Soufi, W. Sci Rep 8, 1783 (2018)
N. Okamura, S. Furumoto, M. T. Fodero-Tavoletti, R. S. Mulligan, R. Harada, P. Yates, et al., Non-invasive assessment of Alzheimer's disease neurofibrillary pathology using F-18-THK5105 PET, Brain 137 (2014) 1762-1771.
Ohmi, K., Kudo, L. C., Ryazantsev, S., et al. (2009) PNAS, 106, 8332-8337.
Pearlman D A, Case D A, Caldwell J W, Ross W S, Cheatham T E, DeBolt S, Ferguson D, Seibel G, Kollman P: AMBER, a package of computer programs for applying molecular mechanics, normal mode analysis, molecular dynamics and free energy calculations to simulate the structural and energetic properties of molecules. Comput Phys Commun 1995, 91:1-41.
C. J. Roberts, Biotechnol. Bioeng., 2007, 98, 927-938.
Daniel R. Roe and Thomas E. Cheatham, Ill, “PTRAJ and CPPTRAJ: Software for Processing and Analysis of Molecular Dynamics Trajectory Data”. J. Chem. Theory Comput., 2013, 9 (7), pp 3084-3095.
rcsb.org H. M. Berman, J. Westbrook, Z. Feng, G. Gilliland, T. N. Bhat, H. Weissig, I. N. Shindyalov, P. E. Bourne. (2000) The Protein Data Bank Nucleic Acids Research, 28: 235-242.
Ryckaert J P, Ciccotti G, Berendsen H J C: Numerical integration of the cartesian equations of motion of a system with constraints: Molecular dynmaics of n-alkanes. J Comput Phys 1977, 23:327-341.
Sandberg, L. M. Luheshi, S. Söllvander, T. Pereira de Barros, B. Macao, T. P. J. Knowles, H. Biverstal, C. Lendel, F. Ekholm-Petterson, A. Dubnovitsky, L. Lannfelt, C. M. Dobson and T. Hard, Proc. Natl. Acad. Sci. USA, 2010, 107, 15 595-15 600.
Sawaya M R, Sambashivan S, Nelson R, Ivanova M I, Sievers S A, et al. (2007) Atomic structures of amyloid cross-beta spines reveal varied steric zippers. Nature 447: 453-457.
Schafmeister CEAF, Ross W S, Romanovski V. LEaP. 1995. University of California, San Francisco.
Schelter, Bjoern O. et al. ‘Concentration-Dependent Activity of Hydromethylthionine on Cognitive Decline and Brain Atrophy in Mild to Moderate Alzheimer's Disease’. 1 Jan. 2019: 931-946.
Schmidt, M. W.; Baldridge, K. K.; Boatz, J. A.; Elbert, S. T.; Gordon, M. S.; Jensen, J. H.; Koseki, S.; Matsunaga, N.; Nguyen, K. A.; S., S.; Windus, T. L.; Dupuis, M.; Montgomery, J. A. J. General atomic and molecular electronic structure system. J. Comput. Chem. 1993, 14 (11), 1347-1363.
Sergeant, N., Sablonniere, B., Schraen-Maschke, S., et al. (2001) Human Molecular Genetics, 10, 2143-2155
Serio T R, Cashikar A G, Kowal A S, Sawicki G J, Moslehi J J, et al. (2000). Science 289: 1317-1321.
Sievers S A, et al. Structure-based design of non-natural amino-acid inhibitors of amyloid fibril formation. Nature. 2011; 475:96-100; PubMed: 21677644
J. D. Sipe, Annu. Rev. Biochem., 1992, 61, 947-975.
von Bergen M, Friedhoff P, Biernat J, Heberle J, Mandelkow E M, et al. (2000) Assembly of tau protein into alzheimer paired helical filaments depends on a local sequence motif (306vqivyk311) forming beta structure. Proc Natl Acad Sci USA 97: 5129-5134.
von Bergen M, Barghorn S, Li L, Marx A, Biernat J, et al. (2001) Mutations of tau protein in frontotemporal dementia promote aggregation of paired helical filaments by enhancing local beta-structure. J Biol Chem 276: 48165-48174.
Wang J, Wang W, Kollman P A, Case D A: Automatic atom type and bond type perception in molecular mechanical calculations. J. Mol. Graphics Model., 2006, 25:247-260.
Wang J, Wolf R M, Caldwell J W, Kollman P A, Case D A: Development and testing of a general amber force field. J Comput Chem 2004, 25:1157-1174.
G. K. Wilcock, M. M. Esiri, Plaques, tangles and dementia: a quantitative study, J. Neurol. Sci. 56 (1982) 343-356.
G. K. Wilcock, S. Gauthier, G. B. Frisoni, J. Jia, J. H. Hardlund, H. J. Moebius, et al., Potential of low dose leucomethylthioninium bis(hydromethanesulphonate) (LMTM) monotherapy for treatment of mild Alzheimer's disease: cohort analysis as modified primary outcome in a phase 3 clinical trial, J. Alzheimers Dis. 61 (2018) 635-657.
C. M. Wischik, M. Novak, P. C. Edwards, A. Klug, W. Tichelaar, R. A. Crowther, Structural characterization of the core of the paired helical filament of Alzheimer disease, Proc. Natl. Acad. Sci. U.S.A 85 (1988) 4884-4888.
C. M. Wischik, P. C. Edwards, R. Y. Lai, M. Roth, C. R. Harrington, Selective inhibition of Alzheimer disease-like tau aggregation by phenothiazines, Proc. Natl. Acad. Sci. U.S.A 93 (1996) 11213-11218.
Wischik et al. in ‘Neurobiology of Alzheimer's Disease’, 2nd Edition, 2000, Eds. Dawbam, D. and Allen, S. J., The Molecular and Cellular Neurobiology Series, Bios Scientific Publishers, Oxford
Claude M Wischik, Roger T Staff, Damon J Wischik, Peter Bentham, Alison D Murray5, John M D Storey, Karin A Kook, Charles R Harrington. Tau Aggregation Inhibitor Therapy: An Exploratory Phase 2 Study in Mild or Moderate Alzheimer's Disease. J Alzheimers Dis. 2015; 44(2):705-20. doi: 10.3233/JAD-142874.
C. M. Wischik, B. O. Schelter, D. J. Wischik, J. M. D. Storey, C. R. Harrington, Modeling prion-like processing of tau protein in Alzheimer's disease for pharmaceutical development, J. Alzheimers Dis. 62 (2018) 1287-1303, https://doi.org/10.3233/JAD-170727.
Zheng J, et al. Macrocyclic β-Sheet Peptides That Inhibit the Aggregation of a Tau-Protein-Derived Hexapeptide. Journal of the American Chemical Society. 2011; 133:3144-3157. DOI: 10.1021/ja110545h; PubMed: 21319744

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and or to the other particular value. Similarly when values are expressed as approximations, by the use of the antecedent “about,” it will be understood that the particular value forms another embodiment.

1. A method for selecting or designing a compound for modulating the aggregation of a Tau protein or a truncated form thereof, the method comprising using computer-implemented molecular modelling means to:
compare the three-dimensional structure of a candidate compound with a three-dimensional structure of at least a part of the Tau protein comprising amino acids 315-378 of SEQ ID NO:1 or a variant or derivative thereof that is structurally equivalent to said part; and
determine whether the candidate compound is able to simultaneously form non-covalent interactions with two or more of Leu315, Ser341, Glu342, Lys343, Phe346, Lys347, Val350, Ser352, Ile354, Lys369, Ile371, Glu372, Phe378 and Thr373, of SEQ ID NO:1, or equivalent amino acids in a variant or derivative, wherein a candidate compound that is able to form said interactions is predicted to modulate the aggregation of the Tau protein or truncated form thereof.

2. The method of claim 1, comprising determining whether the candidate compound is able to simultaneously form non-covalent molecular interactions with Lys343 and Glu372 of SEQ ID NO:1.

3. The method of claim 1, wherein the compound is for inhibiting the aggregation of a Tau protein or a truncated form thereof into paired helical filaments, and wherein the compound is optionally a small molecule, a peptide, a polypeptide or a combination thereof.

4. (canceled)

5. The method of claim 1, wherein the three-dimensional structure of the part of the Tau protein comprising amino acids 315-378 of SEQ ID NO:1 comprises (i) a binding pocket that is capped by Phe378 and Phe346, exposes the hydrophobic side chains of residues Val350, Leu315, Ile354 and/or Ile371, and contains residues Leu315, Lys343, Lys347, Glu372, and/or Thr373 capable of forming hydrogen bonds to a molecule within the binding pocket;
(ii) a hairpin loop between residues Val337 and Gly355 of SEQ ID NO:1;
(iii) the sequence Pro364-Gly367 located between a loop formed by the sequence Tyr219-Lys331 and the sequence Pro332-Gly335;
(iv) is stabilized at least in part by hydrogen bonds between Glu342 and Val318 and/or Thr319, between Gly367 and Lys340, between Asn368 and Lys340, between Lys369 and Glu372, between Lys370 and Asp358, between Ile371 and Ser316, between Glu372 and Ser356, between Thr373 and Gln351, Ser316, between His374 and Gln351, and between Lys375 and Gln351; and/or
(v) is that represented by the structure co-ordinates in Table 1, or a structure modelled on these coordinates.

6-9. (canceled)

10. The method of claim 1, wherein the three-dimensional structure of the part of the Tau protein is obtainable by performing molecular dynamics simulations of a part of the Tau protein comprising amino acids 315-378 of SEQ ID NO:1 in the presence of LMT to obtain one or more complex conformations and selecting a three-dimensional structure of the part of the Tau protein by applying a stability criterion and a binding affinity criterion to the one or more complex conformations; optionally wherein the stability criterion applies to the distance between complex conformations in consecutive frames of the molecular dynamics simulation after a predetermined amount of time, and/or wherein the binding affinity criterion applies to the value of a placement scoring function <−80 kcal/mol or a scoring function <−8.5 as determined by the GB IV scoring function within the CCG MOE docking software.

11. (canceled)

12. The method of claim 1, further comprising designing a pharmacophore model, wherein the pharmacophore includes features representative of non-covalent molecular interactions with two or more of: Leu315, Ser341, Glu342, Lys343, Phe346, Lys347, Val350, Ser352, Ile354, Lys369, Ile371, Glu372, Phe378 and Thr373, of SEQ ID NO:1.

13. The method of claim 1, wherein the part of the Tau protein:
(i) comprises amino acids 306-378 of SEQ ID NO:1;
(ii) comprises amino acids 297-391 of SEQ ID NO:1;
(ii) comprises amino acids 295-391 of SEQ ID NO:1;
(iv) consists of amino acids 297-391 of SEQ ID NO:1;
(v) consists of amino acids 295-391 of SEQ ID NO:1; or
(vi) consists of amino acids 306-378 of SEQ ID NO:1.

14. (canceled)

15. The method of claim 1, further comprising repeating the steps of comparing and determining with a further candidate compound that differs from the previous candidate compound in at least one substituent.

16. The method of claim 1, wherein comparing the three-dimensional structure of a candidate compound with a three-dimensional structure of at least a part of the Tau protein comprising amino acids 315-378 of SEQ ID NO:1 or a variant or derivative thereof that is structurally equivalent to said region comprises computing the interaction energy between the candidate compound and the part of the Tau protein represented in the three-dimensional structure.

17. A computing system comprising a processor and a memory storing machine-readable instructions that, when executed by the processor, cause the processor to implement the method of claim 1.

18. A computer-implemented method for evaluating the ability of a candidate compound to bind to a binding pocket of a Tau protein, the method comprising the steps of:
(a) receiving the three-dimensional structure coordinates of a part of the Tau protein comprising amino acids 315-378 of SEQ ID NO:1 or equivalent amino acids in a variant or derivative thereof that is structurally equivalent to said part, wherein the three-dimensional structure of amino acids 315-378 of SEQ ID NO: 1 comprises the binding pocket of the Tau protein;
(b) performing a fitting operation between a candidate compound and the binding pocket; and
(c) analysing the results of the fitting operation to determine whether the candidate compound is able to bind to the binding pocket.

19. The method of claim 18, wherein the three-dimensional structure of the part of the Tau protein comprising amino acids 315-378 of SEQ ID NO:1 is that represented by the structure co-ordinates shown in Table 1, or a structure modelled on these coordinates.

20. The method of claim 18, wherein the candidate compound is able to bind to the pocket if it is able to simultaneously form non-covalent molecular interactions with two or more of Leu315, Ser341, Glu342, Lys343, Phe346, Lys347, Val350, Ser352, Ile354, Lys369, Ile371, Glu372, Phe378 and Thr373, of SEQ ID NO:1; optionally with Lys343 and Glu372 of SEQ ID NO:1.

21. (canceled)

22. The method of claim 18, wherein the binding pocket is capped by Phe378 and Phe346, exposes the hydrophobic side chains of residues Val350, Leu315, Ile354 and/or Ile371, and contains residues Leu315, Lys343, Lys347, Glu372, and/or Thr373 capable of forming hydrogen bonds to a molecule within the binding pocket.

23. The method of claim 18, wherein the three-dimensional structure of the part of the Tau protein comprising amino acids 315-378 of SEQ ID NO:1
(i) comprises a hairpin loop between residues Val337 and Gly355 of SEQ ID NO:1;
(ii) comprises the sequence Pro364-Gly367 located between a loop formed by the sequence Tyr219-Lys331 and the sequence Pro332-Gly335; or
(iii) is stabilised at least in part by hydrogen bonds between Glu342 and Val318, Thr319, between Gly367 and Lys340, between Asn368 and Lys340, between Lys369 and Glu372, between Lys370 and Asp358, between Ile371 and Ser316, between Glu372 and Ser356, between Thr373 and Gln351, Ser316, between His374 and Gln351, between Lys375 and Gln351.

24-25. (canceled)

26. The method of claim 18, wherein the three-dimensional structure coordinates of a part of the Tau protein comprising amino acids 315-378 of SEQ ID NO:1 or equivalent amino acids in a variant or derivative thereof that is structurally equivalent to said part have been obtained by performing molecular dynamics simulations of a part of the Tau protein comprising amino acids 315-378 of SEQ ID NO:1 in the presence of LMT to obtain one or more complex conformations and selecting a complex conformation using a stability criterion and a binding affinity criterion.

27. The method of claim 18, wherein step (a) receiving the three-dimensional structure coordinates of a part of the Tau protein comprises obtaining the three-dimensional structure coordinates of a part of the Tau protein comprising amino acids 315-378 of SEQ ID NO:1 or equivalent amino acids in a variant or derivative thereof that is structurally equivalent to said part by:
(a) performing molecular dynamics simulations of a part of the Tau protein comprising amino acids 315-378 of SEQ ID NO:1 in the presence of LMT to obtain one or more complex conformations that differ in their three-dimensional conformations; and
(b) selecting a complex conformation using a stability criterion and a binding affinity criterion, wherein the three-dimensional structure coordinates of a part of the Tau protein comprising amino acids 315-378 of SEQ ID NO:1 are defined as the three-dimensional structure coordinates of the part of the Tau protein in the selected complex conformation;
optionally wherein the stability criterion applies to the distance between conformations in consecutive frames of the molecular dynamics simulation after a predetermined amount of time, and/or wherein the binding affinity criterion applies to the value of a docking score.

28. The method of claim 18, wherein the part of the Tau protein comprises amino acids 306-378 of SEQ ID NO:1, wherein the part of the Tau protein comprises amino acids 297-391 of SEQ ID NO:1, wherein the part of the Tau protein comprises amino acids 295-391 of SEQ ID NO:1, wherein the part of the Tau protein consists of amino acids 297-391 of SEQ ID NO:1, wherein the part of the Tau protein consists of amino acids 295-391 of SEQ ID NO:1, or wherein the part of the Tau protein consists of amino acids 306-378 of SEQ ID NO:1.

29. The method of claim 18, wherein the three-dimensional structure coordinates of the part of the Tau protein comprising amino acids 315-378 of SEQ ID NO:1 or equivalent amino acids in a variant or derivative thereof that is structurally equivalent to said part correspond to a conformation that has the following structural characteristics:
hydrogen bonds between Glu342 and Val318 and/or Thr319; optionally wherein the hydrogen bonds are between the Glu342 carboxylic acid and the backbone NH of Val318 and the sidechain OH of Thr319;
one or more hydrogen bonds between one or more of residues Lys369-Thr377 and one or more of residue Ser341-Gln351; optionally wherein the one or more bonds comprise:
(i) a bond between Gln351 and Thr373, preferably wherein the bond is between the backbone carbonyl of Gln351 and the hydroxyl sidechain of Thr373;
(ii) a bond between Gln351 and His374, preferably wherein the bond is between the sidechain carbonyl of Gln351 and the sidechain amine of His374;
(iii) a bond between Gln351 and Lys375, preferably wherein the bond is between the sidechain carbonyl of Gln351 and the backbone amine of Lys375;
(iv) a bond between Arg349 and Thr377, preferably wherein the bond is between a sidechain amine of Arg349 and the hydroxyl sidechain of Thr377, between a sidechain amine of Arg349 and the hydroxyl backbone of Thr377, and/or between the carbonyl backbone of Arg349 and the hydroxyl sidechain of Thr377;
(v) a bond between Glu372 and Ser356, preferably wherein the bond is between the carboxylic acid side chain of Glu372 and the backbone NH of Ser356, or between the carboxylic acid side chain of Glu372 and the OH-sidechain of Ser356; and/or
(vi) a bond between Glu372 and Lys369, preferably wherein the bond is between the carboxylic acid side chain of Glu372 and the NH sidechain of Lys369;
no beta sheets;
a hairpin loop comprising residues Val337-Gly355;
the PGGG sequence formed by residues Pro364-Gly367 is within a distance of 13 A of the PGGG sequence Pro332-Gly335 and/or within a distance of 2 A of a loop formed by the sequence Thr319-Lys331;
the PGGG sequence formed by residues Pro364-Gly367 is located between the PGGG sequence Pro332-Gly335 and a loop formed by the sequence Thr319-Lys331;
residues Lys369-Thr377 are within a distance of 6 Å of residues Asp314-Ser316, optionally wherein the distance between the Ser316 beta-carbon and Thr373 backbone carbonyl is between 2.5 Å and 5.0 Å;
residues Gly355-Gly367 and Asn368-Arg379 are within 2 A hydrogen bonding distance;
Glu338 is folded towards Val363, optionally wherein the distance (RMSD) between the carbonyl oxygen side chain of Glu338 and the backbone amine nitrogen of Val363 NH during the final 10 ns of a 50 ns simulation is between 2 Å and 4 Å, or below 5 Å;
the total water accessible surface area calculated for the part of the protein is at least 20% lower than the corresponding values calculated for a conformation as provided by the three-dimensional coordinates with PDB identifier 5O3L; and/or
the polar and/or hydrophobic accessible surface area(s) calculated for the part of the protein is/are at least 20% lower than the corresponding values calculated for a conformation as provided by the three-dimensional coordinates with PDB identifier 5O3L.

30. A method for selecting or designing a compound for modulating the aggregation of a Tau protein or a truncated form thereof, the method comprising:
using a three-dimensional structural model of at least a part of the Tau protein comprising amino acids 315-378 of SEQ ID NO:1 or a variant or derivative thereof that is structurally equivalent to said part, wherein the model is an intermediate in the aggregation process of the part of the Tau protein with a paired helical filament (PHF), wherein the model is generated by simulating the conformational changes of the part of the Tau protein from a compact folded state to a an aggregated state such that:
(i) residues Val337-Gln355 form a hairpin loop that moves to align with alternating positively charged and negatively charged sidechain stacks in the hairpin loop of the PHF;
(ii) residue Pro332 switches between a trans and a cis configuration;
(iii) residues 355-378 and 306-318 move to form stabilising cross-β sheets with corresponding residues of the PHF through hydrophobic zippering; and
generating a model of a complex of the compound and the intermediate.

31. The method of claim 30, wherein the compact folded state has
(i) any of the structural characteristics defined in claim 29 and/or
(ii) the structure co-ordinates shown in Table 1, or a structure modelled on these coordinates.

32. (canceled)

33. The method of claim 30, wherein generating a model of a complex of the compound and the intermediate comprises identifying the compound as binding to the intermediate and preventing the occurrence of any of steps (i) to (v), optionally any of steps (i) to (iii).

34. (canceled)


SEQUENCE LISTING (9 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 95 aa, Homo sapiens
IKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAE

SEQ ID NO: 4 - protein, 73 aa, Homo sapiens
VQIVYKPVDLSKVTSKCGSLGNIHHKPGGGQVEVKSEKLDFKDRVQSKIGSLDNITHVPGGGNKKIETHKLTF

SEQ ID NO: 5 - protein, 97 aa, Homo sapiens
DNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAE

SEQ ID NO: 6 - protein, 6 aa, Artificial Sequence
  synthetic construct
VQIVYK

SEQ ID NO: 7 - protein, 6 aa, Artificial Sequence
  synthetic construct
VQIINK

SEQ ID NO: 8 - protein, 4 aa, Artificial Sequence
  synthetic construct
WINK

SEQ ID NO: 9 - protein, 4 aa, Artificial Sequence
  synthetic construct
MINK